(19) United States
(12) Patent Application Publication
(10) Pub. No.: US 2024/0293361 A1
(43) Pub. Date: Sep. 5, 2024

(54) Use of Statins to Treat or Prevent Drug-Induced Hearing Loss

(71) Applicant: The United States of America, as represented by the Secretary, Dept. of Health and Human Services, Bethesda, MD (US)

(72) Inventors: Lisa Lynn Cunningham, Rockville, MD (US); Nicole C. Schmitt, Potomac, MD (US); Katherine Ann Fernandez, Montgomery Village, MD (US)

(21) Appl. No.: 17/791,239

(22) PCT Filed: Jan. 25, 2021
(86) PCT No.: PCT/US2021/014918

§ 371 Date: Jul. 7, 2022

Related U.S. Application Data
(60) Provisional application No. 62/966,794, filed on Jan. 28, 2020.

(51) Int. Cl. A61K 31/40; A61K 33/243; A61P 27/16

(52) U.S. Cl. CPC A61K 31/40; A61P 27/16; A61K 33/243

(57) ABSTRACT

The invention relates to methods and compositions for treating or preventing drug-induced hearing loss.

### CROSS-REFERENCE TO RELATED APPLICATIONS

This application claims the benefit of U.S. Provisional Application No. 62/966,794, filed Jan. 28, 2020, which is incorporated by reference herein in its entirety.

### GOVERNMENT LICENSE RIGHTS

This invention was made with U.S. Government support. The U.S. Government has certain rights in the invention.

### BACKGROUND OF THE INVENTION

### Field of the Invention

This disclosure relates to compositions and methods for treating or preventing drug-induced hearing loss.

### Description of Related Art

Cisplatin is among the most effective and widely-used anti-cancer drugs, used to treat a variety of solid tumors, including testicular, ovarian, bladder, cervical, head and neck, and numerous other malignancies. Due in part to the efficacy of cisplatin (Miller et al., 2019), there are currently an estimated 16.9 million cancer survivors nationwide (Bluethmann et al., 2016). Consequently, there is intense clinical and research interest in issues of survivorship and quality of life for these survivors. Many individuals treated with cisplatin experience significant toxicities, including nephrotoxicity, myelosuppression and ototoxicity. Approximately 60% of adult patients who undergo cisplatin treatment acquire a permanent hearing loss (Frisinia et al., 1984, Bertolini et al., 2004, Coradini et al., 2007, Knight et al., 2017 Marchnitz et al., 2018). Hearing loss is a burdensome side effect for cancer survivors; it compromises daily communication with friends and family and can lead to loneliness, isolation, and frustration (Ciorba et al., 2012). Cisplatin-induced hearing loss is permanent, and there are currently no FDA-approved therapies to prevent or reverse cisplatin ototoxicity.

Statins are hydroxymethylglutaryl-CoA (HMG-COA) reductase inhibitors, a class of drugs used primarily to reduce high cholesterol in individuals at risk for cardiovascular disease. In addition to their effects on HMG Co-A reductase, statins have a variety of pleiotropic effects, including improved endothelial function and microcirculation (Liao et al., 2005), decreased inflammation (Jain et al., 2005, Barbosa et al., 2017, Bao et al., 2018) and reduced oxidative stress (Rodrigues et al., 2019, Zhang et al., 2019). Statins have been associated with decreased risk of both stroke (Collins et al., 2002, Aznaouridis et al., 2019) and central nervous system disorders (Cucchiara et al., 2001, Sparks et al., 2005, Zhang et al., 2005). Overall statins have good safety profiles in humans; however, important side effects of statin use include myopathy, liver dysfunction, and rare cases of rhabdomyolysis (Jahani et al., 2016).

Previous studies in animal models have demonstrated a protective effect of statin administration against hearing loss caused by noise trauma (Park et al., 2012, Whitlon et al., 2015, Jahani et al., 2016, Ritcher et al., 2018, Bao et al., 2018; Barbosa et al., 2017; Jain and Ridker, 2005; Liao, 2005; Rodrigues et al., 2019; Zhang et al., 2020), age-related hearing loss (presbycusis) (Syka et al., 2007, Lee et al., 2016; Shen et al., 2018; Ung et al., 2018a), and aminoglycoside antibiotic-induced hearing loss (Brand et al., 2011, Jahani et al., 2016; Park et al., 2012; Richter et al., 2018; Whitlon et al., 2015). In humans, statin use is associated with improved hearing function in older individuals (Olzowy et al., 2007, Gopinath et al., 2011, Brand et al., 2011, Fernandez et al., 2020), improved auditory sensitivity in subjects with noise-induced hearing loss (Sutbas et al., 2007), and reduced tinnitus (Sutbas et al., 2007, Hameed et al., 2014, Oylumlu et al., 2013, Gopinath et al., 2011; Olzowy et al., 2007) and vestibular dysfunction (Saadah et al., 1993). More recently, the instant inventors have shown that lovastatin reduces cisplatin-induced hearing loss in mice (Fernandez et al., 2020). Mice that received lovastatin during cyclic administration of cisplatin demonstrated reduced hearing loss as measured by auditory brainstem response (ABR) testing (Fernandez et al., 2019). A recent review summarizes the role of statins as otoprotective agents in both animal and human studies (Prayuenyong et al., 2020). Taken together, these data indicate that statin use may be associated with a reduced amount of hearing loss caused by a variety of stressors to the inner ear that can otherwise result in permanent damage. Statins are FDA-approved drugs with good safety profiles in humans.

Given the unmet clinical need for therapies to reduce cisplatin-induced hearing loss, the instant inventors have examined the relationship between statin use and cisplatin-induced hearing loss in patients undergoing cisplatin-based chemoradiation therapy (CRT) to treat head and neck cancer. The instant invention discloses the potential for concomitant use of statin drugs during chemotherapy to reduce or prevent hearing loss in patients undergoing cisplatin therapy to treat head and neck cancer.

### SUMMARY OF THE INVENTION

It is against the above background that the instant invention provides certain advantages over the prior art.

Although this invention as disclosed herein is not limited to specific advantages or functionalities (such as, for example, methods of treatment or prevention of drug-induced hearing loss, kits for treatment or prevention of drug-induced hearing loss, and use of compositions that possess pharmacological activities of atorvastatin), the invention provides a method of reducing or preventing drug-induced hearing loss in an individual receiving the drug, or intended to receive the drug, the method comprising administering to the individual a compound that possesses one or more pharmacological activities of atorvastatin, and/or wherein the compound possesses one or more pharmacokinetic parameters substantially similar to one or more pharmacokinetic parameters of atorvastatin.

In one aspect of the methods disclosed herein, the one or more pharmacokinetic parameters is/are selected from the group consisting of the ability to cross a blood barrier, lipophilicity, half-life, potency, bioavailability, absorption, and excretion.

In one aspect, the methods disclosed herein, the blood-barrier separates the inner ear from peripheral blood.

In one aspect, the methods disclosed herein, the one or more pharmacological activities is/are selected from the group consisting of inhibition of hydroxymethylglutaryl-CoA (HMG-COA) reductase, modulation of endothelial function, reduction of inflammation, induction of heme oxygenase-1 (Hmox-1, and promotion of elongation of spiral ganglion neurons (SGN))

In one aspect of the methods disclosed herein, the compound inhibits hydroxymethylglutaryl-CoA (HMG-COA) reductase.

In one aspect, the methods disclosed herein, the compound is a statin, or a functional derivative thereof.

In one aspect, the methods disclosed herein, the statin, or functional derivative thereof, is administered at a normally prescribed dose.

In one aspect of the methods disclosed herein, the compound is selected from the group consisting of atorvastatin, fluvastatin, lovastatin, pravastatin, rosuvastatin, simvastatin, pitavastatin, and functional derivatives thereof.

In one aspect of the methods disclosed herein, the compound is atorvastatin, or a functional derivative thereof.

In one aspect of the methods disclosed herein, the individual has, or is suspected of having, cancer.

In one aspect of the methods disclosed herein, the cancer is a cancer of the head or neck.

In one aspect of the methods disclosed herein, the drug is selected from the group consisting of a non-steroidal anti-inflammatory agent, an antibiotic, a chemotherapeutic agent, a diuretic, and a quinine-based compound.

In one aspect of the methods disclosed herein, the drug is a chemotherapeutic agent.

In one aspect of the methods disclosed herein, the drug possesses one or more activities of cisplatin or carboplatin.

In one aspect of the methods disclosed herein, the drug is cisplatin, carboplatin, or a functional derivative thereof.

In one aspect of the methods disclosed herein, the compound is administered at a time prior to the individual receiving a first administration of the drug.

In one aspect of the methods disclosed herein, the compound is administered during the time period the individual is administered the drug.

In one aspect of the methods disclosed herein, reducing hearing loss comprises reducing the threshold shift.

The invention also provides a kit for reducing or preventing drug-induced hearing loss in an individual receiving the drug, or intended to receive the drug, the kit comprising: (1) a compound that reduces or prevents the drug-induced hearing loss, wherein the compound possesses one or more pharmacological activities of atorvastatin, and/or wherein the compound comprises one or more pharmacokinetic parameters substantially similar to one or more pharmacokinetic parameters of atorvastatin; and (2) instructions for administering the compound to the individual.

In one aspect of the kits disclosed herein, the one or more pharmacokinetic parameters is/are selected from the group consisting of the ability to cross a blood barrier, lipophilicity, half-life, potency, bioavailability, absorption, and excretion.

In one aspect of the kits disclosed herein, the blood-barrier separates the inner ear from peripheral blood.

In one aspect of the kits disclosed herein, the one or more pharmacological activities is/are selected from the group consisting of inhibition of hydroxymethylglutaryl-CoA (HMG-CoA) reductase, modulation of endothelial function, reduction of inflammation, induction of heme oxygenase-1 (Hmox-1, and promotion of elongation of spiral ganglion neurons (SGN)).

In one aspect of the kits disclosed herein, the compound inhibits hydroxymethylglutaryl-CoA (HMG-COA) reductase.

In one aspect of the kits disclosed herein, the compound is a statin, or a functional derivative thereof.

In one aspect of the kits disclosed herein, the statin, or functional derivative thereof, is administered at a normally prescribed dose.

In one aspect of the kits disclosed herein, the compound is selected from the group consisting of atorvastatin, fluvastatin, lovastatin, pravastatin, rosuvastatin, simvastatin, pitavastatin, and derivatives thereof.

In one aspect of the kits disclosed herein, the compound is atorvastatin, or a functional derivative thereof.

In one aspect, the kits disclosed herein, the individual has, or is suspected of having, cancer.

In one aspect of the kits disclosed herein, the cancer is a cancer of the head or neck.

In one aspect, the kits disclosed herein, the drug is selected from the group consisting of a non-steroidal anti-inflammatory agent, an antibiotic, a chemotherapeutic agent, a diuretic, and a quinine-based compound.

In one aspect of the kits disclosed herein, the drug is a chemotherapeutic agent.

In one aspect of the kits disclosed herein, the drug possesses one or more activities of cisplatin or carboplatin.

In one aspect of the kits disclosed herein, the drug is cisplatin, carboplatin, or a functional derivative thereof.

The invention also provides a use of a compound that possesses one or more pharmacological activities of atorvastatin, and/or that possesses one or more pharmacokinetic parameters substantially similar to one or more pharmacokinetic parameters of atorvastatin, in the preparation of a medicament for reducing or preventing drug-induced hearing loss in an individual.

In one aspect of the uses disclosed herein, the one or more pharmacokinetic parameters is/are selected from the group consisting of the ability to cross a blood barrier, lipophilicity, half-life, potency, bioavailability, absorption, and excretion.

In one aspect of the uses disclosed herein, the blood-barrier separates the inner ear from peripheral blood.

In one aspect of the uses disclosed herein, the one or more pharmacological activities is/are selected from the group consisting of inhibition of hydroxymethylglutaryl-CoA (HMG-CoA) reductase, modulation of endothelial function, reduction of inflammation, induction of heme oxygenase-1 (Hmox-1, and promotion of elongation of spiral ganglion neurons (SGN)).

In one aspect of the uses disclosed herein, the compound inhibits hydroxymethylglutaryl-CoA (HMG-COA) reductase.

In one aspect of the uses disclosed herein, the compound is a statin, or a functional derivative thereof.

In one aspect of the uses disclosed herein, the compound is selected from the group consisting of atorvastatin, fluvastatin, lovastatin, pravastatin, rosuvastatin, simvastatin, pitavastatin, and functional derivatives thereof.

In one aspect of the uses disclosed herein, the compound is atorvastatin, or a functional derivative thereof.

In one aspect of the uses disclosed herein, the individual has, or is suspected of having, cancer.

In one aspect of the uses disclosed herein, the cancer is a cancer of the head or neck.

In one aspect of the uses disclosed herein, the drug is selected from the group consisting of a non-steroidal anti-inflammatory agent, an antibiotic, a chemotherapeutic agent, a diuretic, and a quinine-based compound.

In one aspect of the uses disclosed herein, the drug is a chemotherapeutic agent.

In one aspect of the uses disclosed herein, the drug possesses one or more activities of cisplatin or carboplatin.

In one aspect of the uses disclosed herein, the drug is selected from the group consisting of cisplatin, carboplatin, and functional derivative thereof.

In one aspect of the methods disclosed herein, the compound is administered at a dosage of approximately 10 to 80 mg/day.

In one aspect of the methods disclosed herein, the compound is administered at a dosage of approximately 10 mg/day.

In one aspect of the methods disclosed herein, the compound is administered at a dosage of approximately 20 mg/day.

In one aspect of the methods disclosed herein, the compound is administered at a dosage of approximately 40 mg/day.

In one aspect of the methods disclosed herein, the compound is administered at a dosage of approximately 80 mg/day.

In one aspect of the methods disclosed herein, the compound comprising one or more of a 10 mg atorvastatin dosage unit, a 20 mg atorvastatin dosage unit, a 40 mg atorvastatin dosage unit, and an 80 mg atorvastatin dosage unit.

In one aspect of the methods disclosed herein, the dosage form is a tablet or a capsule.

In one aspect of the methods disclosed herein, the dosage form is a gelatin capsule.

These and other features and advantages of the instant invention will be more fully understood from the following detailed description taken together with the accompanying claims. It is noted that the scope of the claims is defined by the recitations therein and not by the specific discussion of features and advantages set forth in the instant description.

### BRIEF DESCRIPTION OF THE DRAWINGS

The following detailed description of the embodiments of the instant invention can be best understood when read in conjunction with the following drawings, where like structure is indicated with like reference numerals and in which:

FIG. 1 shows a flow diagram of study design and participants. Retrospective and prospective data were combined for analyses. Eligibility criteria were the same in both the retrospective and prospective segments. A total of 277 subjects were included in the analyses.

FIG. 2A-D show atorvastatin use is associated with reduced cisplatin-induced hearing loss. Baseline audiometric thresholds were compared to thresholds obtained following cisplatin treatment to determine threshold shifts. FIG. 2A shows that in subjects not taking a statin (N=324 ears), cisplatin treatment resulted in threshold shifts that were more severe at higher frequencies. Subjects taking any statin (N=219 ears) had significantly less cisplatin-induced hearing loss than subjects who were not taking a statin. Atorvastatin users (N=97 ears) had significantly less cisplatin-induced hearing loss than non-statin users. In contrast, cisplatin-induced threshold shifts among simvastatin users (N=70 ears) were not significantly different from those of non-statin users. Data represent means±SEM, 2-way ANOVA, Dunnett's multiple comparisons test. FIG. 2B shows atorvastatin dose was not correlated with high frequency (6-12.5 kHz) hearing loss. Each dot represents one ear. Non-statin users (N=324 ears) had 15.9±20.3 dB shifts in high-frequency pure tone average (HF PTA). Atorvastatin users (N=97 ears) had shifts of 7.8±11.8 dB. There was no correlation between atorvastatin dose and threshold shift. Pearson R Correlation. FIG. 2C shows the incidence of cisplatin-induced hearing loss among non-statin users is 48% per CTCAE criteria. Subjects taking any statin had significantly lower incidence of hearing loss than non-statin users. The incidence of hearing loss was further reduced among atorvastatin users. Data are percentage of ears per group. Statistical analysis consisted of Chi-Square. FIG. 2D shows statin use, atorvastatin in particular, is associated with reduced severity of hearing loss. CTCAE criteria were used to categorize the severity of hearing loss. Chi-Square analysis shows a significant difference in the distribution of CTCAE hearing loss grades, where the incidence of a Grade 2 or higher hearing loss is reduced in statin users compared to non-statin users. This difference was even greater for atorvastatin users. Data are percentage of ears per group. * p<0.05, ** p<0.01, *** p<0.001.

FIG. 3 shows atorvastatin use significantly reduces the odds of a clinically meaningful cisplatin-induced hearing loss. An analysis of the incidence of a CTCAE-defined hearing loss due to cisplatin therapy in the full cohort and key subgroups is shown. For the full cohort and the subject subgroups, the difference in the incidence (% of ears) and 95% confidence intervals (CI), were estimated using a nonlinear mixed effect analysis, fitting the Poisson model. Significant differences (red diamonds) in the calculated incidence of a CTCAE grade 1 or higher hearing loss were observed for the full cohort as well as for the male subgroup and for those receiving higher (>200 mg/m2) cumulative cisplatin dose, radiation, and those with mild hearing loss at baseline. CNT=could not test, due to insufficient sample size in “atorvastatin+cisplatin therapy” comparison group.

FIG. 4A-B show 3-year overall survival and disease-free survival are not different among statin users, atorvastatin users, and those not taking a statin. Kaplan Meier estimates of overall (FIG. 4A) and disease free (FIG. 4B) survival are shown. A log-rank (Mantel-Cox) test indicates no significant differences in either overall or disease-free survival among groups, (p>0.05). No statin group, n=107, Any statin group, n=68, Atorvastatin group, n=33.

FIGS. 5A-B show the incidence and severity of cisplatin-induced hearing loss (as defined by TUNE criteria) is reduced among atorvastatin users relative to non-statin users. FIG. 5A shows the incidence of cisplatin-induced hearing loss is 52% per TUNE criteria amongst non-statin users (black). Subjects taking any statin (blue bar) had significantly lower incidence of cisplatin-induced hearing loss than non-statin users. The incidence of hearing loss was further reduced among atorvastatin users to 34% (orange bar). Data are percent of ears per group. Statistical analysis consisted of Chi-Square, ** p<0.01. FIG. 5B shows statin use, atorvastatin in particular, is associated with reduced severity of hearing loss. TUNE scale criteria were used to categorize the severity of cisplatin-induced hearing loss. Subjects taking any statin had significantly reduced incidence of a Grade 2 or higher hearing loss compared to non-statin users. This difference was even greater for atorvastatin users. Data are percentage of ears per group. Statistical analysis consisted of Chi-Square, *p<0.05, *** p<0.001.

FIG. 6 shows the cochlear radiation dose is not correlated with changes in high frequency hearing sensitivity. Plotted are high frequency (6 to 12.5 kHz) threshold shifts and the mean cochlear radiation dose for each ear in the prospective cohort. Pearson r and Spearman correlation, p>0.05. N=56 ears.

FIG. 7 shows a Phase 3 study overview (see also, Example 2).

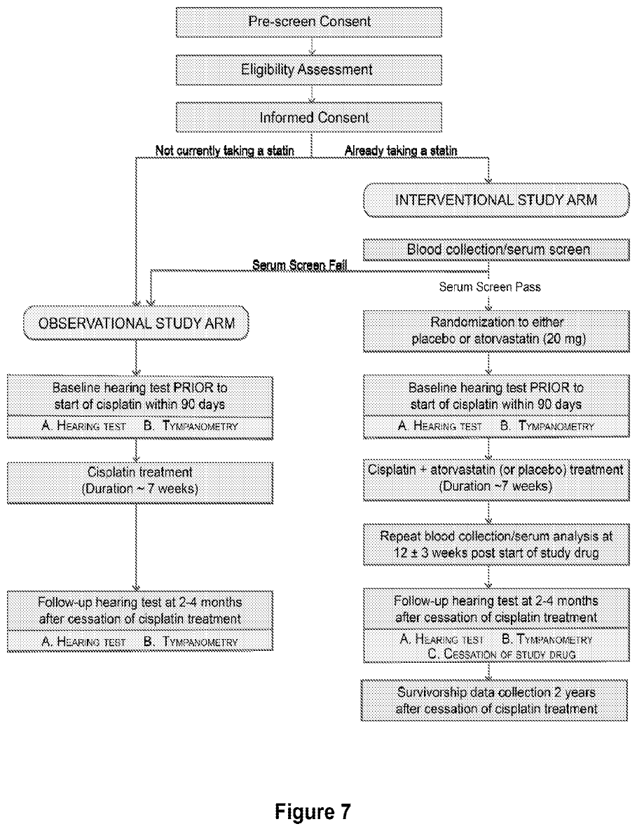

FIG. 8 shows justification of dose. Data collected from 50 atorvastatin users (97 ears) at doses ranging from 10-80 mg tablets indicate that all doses of atorvastatin are equally effective at reducing cisplatin-induced hearing loss in individuals with head and neck cancer. A low dose (20 mg) was selected to reduce the incidence of potential side effects. The study is designed to utilize 20 mg atorvastatin based on the statistically significant and clinically powered (p>0.8) data from the retrospective/observational prospective study.

FIG. 9 shows that based on a chi-square analysis and a linear regression analysis of the preliminary data from retrospective/observational prospective study (n=277 subjects with head and neck cancer obtained in collaboration with University of Rochester Medical Center, Walter Reed National Military Medical Center, and Johns Hopkins University) controlling for cumulative cisplatin dose, sex, age, radiation, and pre-existing hearing loss, a statistical significance was determined using CTCAEv5.0 and TUNE criteria to compare changes in hearing in atorvastatin users and non-statin users (p<0.03, power >0.8).

Skilled artisans will appreciate that elements in the Figures are illustrated for simplicity and clarity and have not necessarily been drawn to scale. For example, the dimensions of some of the elements in the Figures can be exaggerated relative to other elements to help improve understanding of the embodiment(s) of the instant invention.

### DETAILED DESCRIPTION OF THE INVENTION

All publications, patents and patent applications cited herein are hereby expressly incorporated by reference for all purposes.

Before describing the instant invention in detail, a number of terms will be defined. As used herein, the singular forms “a,” “an,” and “the” include plural referents unless the context clearly dictates otherwise. For example, reference to a “statin” means one or more statins.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the instant invention.

For the purposes of describing and defining the instant invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

Throughout this specification, unless the context specifically indicates otherwise, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising”, “consisting essentially of”, and “consisting of” may be replaced with either of the other two terms, while retaining their ordinary meanings.

As utilized in accordance with the instant disclosure, unless otherwise indicated, all technical and scientific terms shall be understood to have the same meaning as commonly understood by one of ordinary skill in the art.

Percentages disclosed herein can vary in amount by +10, 20, or 30% from values disclosed and remain within the scope of the contemplated disclosure.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein and in the drawings, ranges and amounts can be expressed as “about” a particular value or range. About also includes the exact amount. For example, “about 5%” means “about 5%” and also “5%.” The term “about” can also refer to +10% of a given value or range of values. Therefore, about 5% also means 4.5%-5.5%, for example.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

As used herein, the term “statin” refers to a 3-hydroxy-3-methylglutaryl coenzyme A (HMG-COA) reductase inhibitor. Statins block the rate-limiting step in de novo cholesterol biosynthesis, namely, the conversion of HMG-COA into mevalonate by HMG-COA reductase. Statins are used primarily as cholesterol-lowering (specifically, low-density lipoprotein (LDL)-lowering) medications to treat hyperlipidemias, such as hypercholesterolemia. Examples of statins and typical daily adult dose ranges provided in parentheses include: atorvastatin (10-80 mg), fluvastatin (20-80 mg), lovastatin (10-80 mg), pitavastatin (1-4 mg), pravastatin (10-80 mg), rosuvastatin (5-40 mg), and simvastatin (5-80 mg).

In some embodiments of the methods disclosed herein, the statin is atorvastatin, cerivastatin, fluvastatin, lovastatin, mevastatin, pitavastatin, pravastatin, rosuvastatin, or simvastatin. In some embodiments, the statin is atorvastatin.

As used herein, the term “atorvastatin” refers to, but is not limited to, atorvastatin calcium, atorvastatin magnesium, atorvastatin aluminum, atorvastatin iron, atorvastatin zinc, and other suitable salts of atorvastatin.

In some embodiments of the kits disclosed herein, the statin is atorvastatin, cerivastatin, fluvastatin, lovastatin, mevastatin, pitavastatin, pravastatin, rosuvastatin, or simvastatin. In some embodiments, the statin is atorvastatin.

In some embodiments of the uses disclosed herein, the statin is atorvastatin, cerivastatin, fluvastatin, lovastatin, mevastatin, pitavastatin, pravastatin, rosuvastatin, or simvastatin. In some embodiments, the statin is atorvastatin.

As used herein, the terms “side effect,” “peripheral effect,” and “secondary effect” are interchangeable and refer to effects or symptoms caused by a drug, medication, or pharmaceutical other than its primary, intended effect or indication.

As used herein, the term “alleviate” refers to the amelioration or lessening of the severity of a side effect or symptom or substantially eliminating said side effect or symptom.

As used herein, the term “treating” or “treatment” refers to the treatment of a disease or disorder described herein, in a subject, preferably a human, and includes inhibiting, relieving, ameliorating, or slowing progression of one or more symptoms of the disease or disorder.

As used herein, the term “therapeutically effective amount” refers to the amount of a drug compound, or pharmaceutically acceptable salt thereof, that alone and/or in combination with other drugs provides benefit in preventing, treating, and/or managing one or more conditions to that may benefit from the properties of that particular drug without causing significant negative or adverse side effects to the target, (1) delaying or preventing the onset of ototoxicity; or (2) slowing down or stopping the progression, aggravation, or deterioration of one or more symptoms of ototoxicity. A therapeutically effective amount may be administered prior to the onset of ototoxicity, for a prophylactic or preventive action. Alternatively or additionally, the therapeutically effective amount may be administered after initiation of ototoxicity, for preventing a worsening of ototoxicity. The therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein, “dosage regimen” refers to the schedule of doses of a therapeutic agent per unit of time, including: the time between doses or the time when the dose(s) are to be given, and the amount of a medicine to be given at each specific time.

As used herein, the term “subject” refers to a warm-blooded animal such as a mammal, preferably a human, which is afflicted with, or has the potential to be afflicted with one or more diseases and disorders described herein.

As used herein, the term “individual” refers to a warm-blooded animal such as a mammal, preferably a human, which is afflicted with, or has the potential to be afflicted with one or more diseases and disorders described herein.

As used herein, the term “patient” is intended to include human and non-human animals, particularly mammals.

As used herein, the term “mammal” refers to both humans and non-humans and include but is not limited to humans, non-human primates, canines, felines, murines, bovines, equines, and porcines.

As used herein, the terms “prevent”, “preventing”, “prevention”, “prophylactic treatment” and the like refer to reducing the probability of developing a disease, disorder, or condition in a subject, who does not have, but is at risk of or susceptible to developing a disease, disorder, or condition. Thus, in some embodiments, an agent can be administered prophylactically to prevent the onset of a disease, disorder, or condition, or to prevent the recurrence of a disease, disorder, or condition.

As used herein, the terms “reducing” and “inhibiting” have their commonly understood meaning of lessening or decreasing.

As used herein, the term “progression” means increasing in scope or severity, advancing, growing or becoming worse.

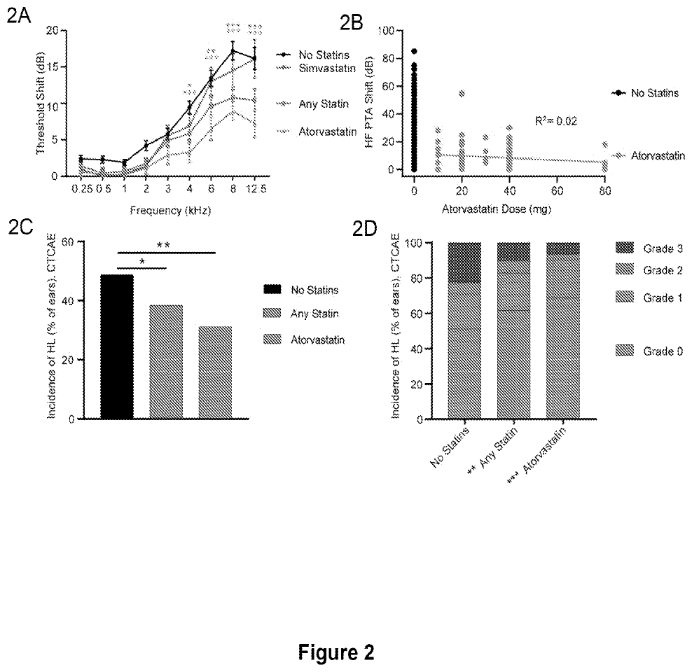

As used herein, the term “half-life” or “absorption half-life” refers to the time required for 50% of a drug to be absorbed following administration to a subject. For example, atorvastatin is an attractive option for individuals with statin intolerance, since its longer half-life allows some users to adopt an alternate-day (Adhyaru and Bhavin, 2018) or biweekly (Ghattas and Pimenta, 2007, Christou et al., 2014) dosing schedule. In the instant invention, all doses of atorvastatin were equally associated with reduced hearing loss (10-80 mg; R²=0.0246) (see e.g., Example 1 and FIG. 2B).

As used herein, the term “bioavailability” as defined in 21 CFR Section 320.1 refers to the rate and extent to which the active ingredient or active moiety is absorbed from a drug product and becomes available at the site of action. Bioavailability data for a particular formulation provides an estimate of the fraction of the administered dose, for example, an oral tablet, that is absorbed into the systemic circulation.

As used herein, the term “absorption” refers to the process of a substance, such as a drug, entering the bloodstream. Absorption can be measured by pharmacokinetic parameters, such as AUCinf and Cmax.

As used herein, the term “administer” or “administration” refers to oral (“po”) administration, administration as a suppository, topical contact, intravenous (“iv”), intraperitoneal (“ip”), intramuscular (“im”), intralesional, intranasal or subcutaneous (“sc”) administration, or the implantation of a slow-release device, e.g., a mini-osmotic pump, to an individual. Administration can be by any route including parenteral and transmucosal (e.g., oral, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, and equivalent methods and modalities know to those of skill in the art.

As used herein, the term “p-value” or “p” refers to a number between 0 and 1 relating to the significance of results obtained. A small p-value indicates strong evidence against the null hypothesis (i.e., the hypothesis that there is no effect), for example≤0.1, indicates statistical significance, with p<0.001 being statistically highly significant (less than one in a thousand chance of being wrong).

As used herein, the term “drug-induced hearing loss” refers to hearing loss caused to the inner ear or auditory nerve, leading to leading to sensorineural deafness. For example, many individuals treated with cisplatin experience significant toxicities, including nephrotoxicity, myelosuppression and ototoxicity. Approximately 60% of adult patients who undergo cisplatin treatment acquire a permanent hearing loss (Frisinia et al., 1984, Bertolini et al., 2004, Coradini et al., 2007, Knight et al., 2017 Marchnitz et al., 2018).

As used herein, the term “adverse event,” “suspected adverse event” refers to any untoward medical occurrence associated with the use of an intervention in humans that is potentially, probably, or definitely related to intervention, as defined in Section 8.4.3.2 (21 CFR 312.32 (a)). For example, adverse events that are expected as a result of HNSCC, cisplatin and/or radiation include, but are not limited to, nausea, vomiting, mucositis, peripheral neuropathy, dysphagia, xerostomia, fatigue, and pain at a tumor site. Adverse events may be classified as: 1) Mild: which is described as events that require minimal or no treatment and do not interfere with the participant's daily activities; 2) Moderate: which is described as events that result in a low level of inconvenience or concern with the therapeutic measures. Moderate events may cause some interference with functioning; 3) Severe: which is described as events that interrupt a participant's usual daily activity and may require systemic drug therapy or other treatment. Severe events are usually potentially life-threatening or incapacitating. Of note, the term “severe” does not necessarily equate to “serious”. The relationship of an adverse event to the study intervention may be classified as: 1) “Definitely Related”, which means there is clear evidence to suggest a causal relationship, and other possible contributing factors can be ruled out. The clinical event, including an abnormal laboratory test result, occurs in a plausible time relationship to study intervention administration and cannot be explained by concurrent disease or other drugs or chemicals. The response to withdrawal of the study intervention (dechallenge) should be clinically plausible. The event must be pharmacologically or phenomenologically definitive, with use of a satisfactory rechallenge procedure if necessary; 2) “Probably Related”, which means there is evidence to suggest a causal relationship, and the influence of other factors is unlikely. The clinical event, including an abnormal laboratory test result, occurs within a reasonable time after administration of the study intervention, is unlikely to be attributed to concurrent disease or other drugs or chemicals, and follows a clinically reasonable response on withdrawal (dechallenge). Rechallenge information is not required to fulfil this definition; 3) Potentially Related which means there is some evidence to suggest a causal relationship (e.g., the event occurred within a reasonable time after administration of the trial medication). However, other factors may have contributed to the event (e.g., the participant's clinical condition, other concomitant events). Although an AE may rate only as “possibly related” soon after discovery, it can be flagged as requiring more information and later be upgraded to “probably related” or “definitely related”, as appropriate; 4) Unlikely to be related which means a clinical event, including an abnormal laboratory test result, whose temporal relationship to study intervention administration makes a causal relationship improbable (e.g., the event did not occur within a reasonable time after administration of the study intervention) and in which other drugs or chemicals or underlying disease provides plausible explanations (e.g., the participant's clinical condition, other concomitant treatments); and 5) Not Related which means the AE is completely independent of study intervention administration, and/or evidence exists that the event is definitely related to another etiology. There must be an alternative, definitive etiology documented by the clinician.

As used herein, the term “serious adverse event” or “SAE” refers to death, a life-threatening adverse event, inpatient hospitalization or prolongation of existing hospitalization, a persistent or significant incapacity or substantial disruption of the ability to conduct normal life functions, or a congenital anomaly/birth defect. Important medical events that may not result in death, be life-threatening, or require hospitalization may be considered serious when, based upon appropriate medical judgment, they may jeopardize the participant and may require medical or surgical intervention to prevent one of the outcomes listed in this definition. Examples of such medical events include allergic bronchospasm requiring intensive treatment in an emergency room or at home, blood dyscrasias or convulsions that do not result in inpatient hospitalization, or the development of drug dependency or drug abuse.

As used herein, the term “risk reduction” and “risk of” refer to the relative risk unless specified to mean absolute risk.

As used herein, the term “unanticipated problems” or “UP” refers to any incident, experience, or outcome that meets all of the following criteria: 1) Unexpected in terms of nature, severity, or frequency given (a) the research procedures that are described in the protocol-related documents, such as the Institutional Review Board (IRB)-approved research protocol and informed consent document; and (b) the characteristics of the participant population being studied; and 2) Related or possibly related to participation in the research (“possibly related” means there is a reasonable possibility that the incident, experience, or outcome may have been caused by the procedures involved in the research); and 3) Suggests that the research places participants or others (which many include research staff, family members or other individuals not directly participating in the research) at a greater risk of harm (including physical, psychological, economic, or social harm) than was previously known or expected.

As used herein, the term “ototoxicity” refers to lesions to the ear induced by ototoxic agents or environmental ototoxic conditions (i.e., agents or conditions toxic to the ear), specifically lesions of the cochlea and/or of the vestibule. Examples of symptoms of ototoxicity include, but are not limited to, hearing loss, balance loss, tinnitus, vertigo and dizziness. As used herein, ototoxicity may include (1) cochlear toxicity which results in hearing loss and tinnitus and (2) vestibular toxicity which results in loss of balance. The mechanism of ototoxicity may include the lesion or destruction of neurons and/or sensory cells (hair cells) and/or endolymph producing cells of the cochlea or the vestibule. The definition and criteria for ototoxicity has been established by the American Speech-Language-Hearing Association (ASHA), the National Cancer Institute (NCI) Common Terminology Criteria for Adverse Events (CTCAE) Ototoxicity Grades, and the TUNE grading system (Thenuissen et al., 2014), and Brock's Hearing Loss Grades. These grading scales indicate a change in hearing that is clinically meaningful to an individual. They differ from each other by the minimum change in hearing necessary to define a clinically-meaningful change and by the specific frequencies used to make such conclusions. ASHA defines a meaningful change by a >10 dB threshold shift at any two consecutive frequencies or a 20 dB change at a single frequency, spanning all frequencies tested. TUNE uses similar frequencies but assigns a minimum change in hearing as an average >10 dB threshold shift across either 1-4 kHz or 6-12 kHz. CTCAE uses threshold shift data spanning 1 to 8 kHz and indicates a meaningful change as an average shift ≥15 dB at any 2 consecutive frequencies. The Brock's Hearing Loss Grades, originally designed for children receiving platinum based therapeutics, are: Grade 0: Hearing thresholds <40 dB at all frequencies; Grade 1: thresholds 40 dB or greater at 8000 Hz; Grade 2: thresholds 40 dB or greater at 4000-8000 Hz; Grade 3: thresholds 40 dB or greater at 2000-8000 Hz; Grade 4: thresholds at 40 dB or greater at 1000-8000 Hz.

As used herein, the term “cochlea” refers to the ventral region of the inner ear, containing the organ of Corti that comprises mechanosensory hair cells and supporting cells. The cochlea is dedicated to the auditory function, converting sound pressure patterns from the outer ear into electrochemical impulses which are passed on to the brain via the auditory nerve.

As used herein, the term “co-administer” refers to administering more than one pharmaceutical agent to a patient. In some embodiments, co-administered pharmaceutical agents are administered together in a single dosage unit. In some embodiments, co-administered pharmaceutical agents are administered separately. In some embodiments, co-administered pharmaceutical agents are administered at the same time. In some embodiments, co-administered pharmaceutical agents are administered at different times.

The term “pharmaceutical formulation” refers to a preparation which is in such form as to permit a biological activity of an active ingredient to be effective, and which contains no additional components which are unacceptably toxic to a subject to which the formulation would be administered.

The pharmaceutical composition can contain formulation materials for modifying, maintaining, or preserving, for example, the pH, osmolarity, viscosity, clarity, color, isotonicity, odor, sterility, stability, rate of dissolution or release, adsorption, or penetration of the composition. Suitable formulation materials include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; triton; tromethamine; lecithin; cholesterol or tyloxapal), stability enhancing agents (such as sucrose or sorbitol), tonicity enhancing agents (such as alkali metal halides-preferably sodium or potassium chloride- or mannitol sorbitol), delivery vehicles, diluents, excipients and/or pharmaceutical adjuvants (see, e.g., REMINGTON'S PHARMACEUTICAL SCIENCES (18th Ed., A. R. Gennaro, ed., Mack Publishing Company 1990), and subsequent editions of the same, incorporated herein by reference for any purpose).

As used herein, the term “placebo” refers to an ostensibly pharmaceutical formulation which lacks a pharmaceutically active ingredient, or lacks the particular pharmaceutical ingredient of interest in a particular study. In the experiments disclosed herein, “placebo” refers to a formulation identical to the formulation given to test subjects but lacking the pharmaceutically active ingredient. In general, a placebo may include inert compounds, and any pharmaceutically acceptable compound which may be found in a medicament, so long as it lacks a pharmaceutically active ingredient (as determined with respect to the pharmaceutical ingredient to which it is to be compared).

As used herein, the term “disease” or “disorder” refers to any condition that damages or interferes with the normal function of a cell, tissue, or organ.

### Cisplatin

Cisplatin is a widely-used and effective drug used to treat adult and pediatric cancers. Due in part to the efficacy of cisplatin (Miller et al., 2019), there are currently an estimated 16.9 million cancer survivors nationwide (Bluethmann et al., 2016). Therefore, there is intense clinical and research interest in issues of survivorship and quality of life for these survivors. However, it is the most ototoxic drug in clinical use, resulting in significant permanent hearing loss in over 50% of treated patients. Other than otoxicity, many individuals treated with cisplatin also experience significant other toxicities, including nephrotoxicity and myelosuppression. Approximately 60% of adult patients who undergo cisplatin treatment acquire a permanent hearing loss (Frisinia et al., 1984, Bertolini et al., 2004, Coradini et al., 2007, Knight et al., 2017 Marchnitz et al., 2018), which compromises daily communication with friends, family, and healthcare providers and can lead to loneliness, social isolation, and frustration (Ciorba et al., 2012). Cisplatin-induced hearing loss is permanent, and there are currently no FDA-approved therapies to reduce or prevent cisplatin ototoxicity. Hence, there is a major need for therapies that reduce cisplatin-induced hearing loss.

### Statins

Statins, commonly known as HMG-COA reductase inhibitors, are FDA-approved drugs that are used primarily to treat hyperlipidemia in individual as risk for cardiovascular disease and are the most effective lipid-lowering drugs currently available. Specifically, they are a class of drugs used to lower cholesterol levels by inhibiting the enzyme HMG-COA reductase, which catalyzes the rate-limiting conversion of HMG-COA into mevalonate by HMG-COA reductase during de novo cholesterol biosynthesis. They have also been shown to exhibit pleiotropic effects and may have potential uses in the treatment of other conditions, such as diabetes, depression, cancer, osteoporosis, ventricular arrhythmias, peripheral arterial disease, and idiopathic dilated cardiomyopathy. Particularly, their pleiotropic effects, include improved endothelial function and microcirculation (Liao et al., 2005), decreased inflammation (Jain et al., 2005, Barbosa et al., 2017, Bao et al., 2018) and reduced oxidative stress (Rodrigues et al., 2019, Zhang et al., 2019). Additionally, statins have been associated with decreased risk of both stroke (Collins et al., 2002, Aznaouridis et al., 2019) and central nervous system disorders (Cucchiara et al., 2001, Sparks et al., 2005, Zhang et al., 2005).

Previous studies in animal models have demonstrated a protective effect of statin administration against hearing loss caused by noise trauma (Park et al., 2012, Whitlon et al., 2015, Jahani et al., 2016, Ritcher et al., 2018), age-related hearing loss (presbycusis) (Syka et al., 2007), aminoglycoside-induced hearing loss (Brand et al., 2011), and most recently, the instant inventors have shown that cisplatin-induced hearing loss (Fernandez et al., 2020). In humans, statin use is associated with improved hearing function in older individuals (Olzowy et al., 2007, Gopinath et al., 2011), improved auditory sensitivity in subjects with noise-induced hearing loss (Sutbas et al., 2007), and reduced tinnitus (Sutbas et al, 2007, Hameed et al., 2014, Oylumlu et al., 2013) and vestibular dysfunction (Saadah et al., 1993).

### Atorvastatin

Atorvastatin is a widely-used drug FDA approved for hyperlipidemia with a good safety profile in humans. Known benefits of atorvastatin include (from prescribing information): 1) reduced risk of myocardial infarction (MI), stroke, revascularization procedures, and angina in patients without coronary heart disease (CHD), but with multiple risk factors; 2) reduced risk of MI and stroke in patients with type 2 diabetes; 3) reduced risk of non-fatal MI, fatal and non-fatal stroke, revascularization procedures, hospitalization for congestive heart failure (CHF) and angina in patients with CHD; and 4) reduced elevated total-C, LDL-C, apo B, and TG levels and increase in HDL-C levels in patients with primary hyperlipidemia. Potential benefits of atorvastatin may include reduced incidence and/or severity of cisplatin-induced hearing loss (see Examples 1 and 2).

Adverse events associated with statin use are rare. Musculoskeletal side effects are the main concern and a common cause of discontinued statin use. Atorvastatin is an attractive option for individuals with statin intolerance, since its longer half-life allows some users to adopt an alternate-day (Adhyaru and Bhavin, 2018) or biweekly (Ghattas and Pimenta, 2007, Christou et al., 2014) dosing schedule. In the study, all doses of atorvastatin were equally associated with reduced hearing loss (10-80 mg; R²=0.0246). According to the American College of Cardiology (ACC) and American Heart Association (AHA) statin dose guidelines, relative to simvastatin and pravasatin, atorvastatin has a higher dose-potency ratio (Jones et al., 1998, Stone et al., 2014, Adams et al., 2015), suggesting that low-dose atorvastatin may be effective at reducing cisplatin-induced hearing loss. Our data suggest that low-dose atorvastatin is an inexpensive drug with low toxicity that holds potential to reduce cisplatin-induced hearing loss in adult patients without reducing the therapeutic efficacy of cisplatin.

Risk statements taken from the atorvastatin package insert include: 1) The five most common adverse reactions in patients treated with atorvastatin that led to treatment discontinuation and occurred at a rate greater than placebo were: myalgia (0.7%), diarrhea (0.5%), nausea (0.4%), alanine aminotransferase increase (0.4%), and hepatic enzyme increase (0.4%); 2) Rare cases of rhabdomyolysis with acute renal failure secondary to myoglobinuria have been reported with atorvastatin calcium and with other drugs in this class. A history of renal impairment may be a risk factor for the development of rhabdomyolysis; 3) Atorvastatin, like other statins, occasionally causes myopathy, defined as muscle aches or muscle weakness in conjunction with increases in creatine phosphokinase (CPK) values >10 times the upper limit of reference range. The concomitant use of higher doses of atorvastatin with certain drugs such as cyclosporine and strong CYP3A4 inhibitors (e.g., clarithromycin, itraconazole, and HIV protease inhibitors) increases the risk of myopathy/rhabdomyolysis; 4) Statins, like some other lipid-lowering therapies, have been associated with biochemical abnormalities of liver function. Persistent elevations (>3 times the upper limit of normal [ULN] occurring on 2 or more occasions) in serum transaminases occurred in 0.7% of patients who received atorvastatin calcium in clinical trials. The incidence of these abnormalities was 0.2%, 0.2%, 0.6%, and 2.3% for 10, 20, 40, and 80 mg, respectively; 5) It is recommended that liver function tests be performed prior to and at 12±3 weeks following the initiation of therapy, and periodically (e.g., semiannually) thereafter. Liver enzyme changes generally occur in the first 3 months of treatment with atorvastatin calcium. Patients who develop increased transaminase levels should be monitored until the abnormalities resolve. Should a clinically significant increase in ALT or AST persist, withdrawal of atorvastatin calcium is recommended; 6) Atorvastatin calcium should be used with caution in patients who consume substantial quantities of alcohol and/or have active liver disease or a history of liver disease, which are potentially increased in HNSCC associated with increased tobacco and alcohol use; 7) Statins interfere with cholesterol synthesis and theoretically might blunt adrenal and/or gonadal steroid production. Clinical studies have shown that atorvastatin calcium does not reduce basal plasma cortisol concentration or impair adrenal reserve. The effects of statins on male fertility have not been studied in adequate numbers of patients. The effects, if any, on the pituitary-gonadal axis in premenopausal women are unknown. Caution should be exercised if a statin is administered concomitantly with drugs that may decrease the levels or activity of endogenous steroid hormones, such as ketoconazole, spironolactone, and cimetidine; and 8) Atorvastatin calcium is contraindicated in women who are or may become pregnant. It is not known whether atorvastatin is excreted in human milk, but a small amount of another drug in this class does pass into breast milk.

On the other hand, given the unmet clinical need for therapies to reduce cisplatin-induced hearing loss, the instant inventors were first to explore the potential benefits of atorvastatin and the relationship between statin use and cisplatin-induced hearing loss in patients undergoing cisplatin-based chemoradiation therapy (CRT) to treat head and neck cancer.

Hearing test data obtained before and after cisplatin therapy from 277 subjects (see e.g., Example 1) were examined using a combined retrospective and prospective observational study design. Hearing test data (audiometric pure-tone thresholds) were obtained within 90 days before onset of CRT and again within 90 days after cessation of cisplatin therapy. Using two established ototoxicity scales, the NCI Common Terminology Criteria for Adverse Events (CTCAE)(NIH National Cancer Institute (2017, www.ctep.cancer.gov) and TUNE (Theunissen et al., 2014), the incidence and severity of cisplatin-induced hearing loss between patients taking a statin compared to those not taking a statin to determine the relationship between statin use and cisplatin-induced hearing loss was compared. Study subjects were assigned to groups based on whether they were taking a statin at the start of CRT.

Significant hearing loss occurred in as many as 48% of subjects, consistent with previous reports (Zuur et al., 2007) (see e.g., Example 1). The incidence of cisplatin-induced hearing loss was significantly reduced in patients who were taking a statin medication concurrently with cisplatin. Specifically, atorvastatin use was associated with both reduced incidence and reduced severity of hearing loss. For example, the incidence of a CTCAE Grade 1 or higher was reduced by 17.6% in atorvastatin users relative to non-statin users (see e.g., Example 1). Moreover, the incidence of a CTCAE Grade 2 or higher, a moderate severity adverse event that may be dose-limiting, was reduced by 19.7% in atorvastatin users relative to non-statin users. Statins are inexpensive drugs with good safety profiles. The data suggest that concurrent use of atorvastatin during cisplatin-based chemotherapy offers an opportunity for reduced hearing loss in these patients without reduced survival.

The mechanisms by which atorvastatin may affect cisplatin-induced hearing loss are unknown. The primary mechanism by which statins control hyperlipidemia is by inhibition of HMG-CoA reductase, the rate-limiting enzyme in the cholesterol synthesis pathway. However, finding that atorvastatin use was associated with reduced cisplatin-induced hearing loss while simvastatin use was not suggests that the protection observed in atorvastatin users may be independent of atorvastatin's effect on HMG Co-A. One explanation for the differences observed between atorvastatin and simvastatin may be differential pharmacokinetics of the two drugs in the inner ear. Like the brain, the inner ear is separated from the peripheral blood flow by a selectively-permeable barrier comprised in part of endothelial cells in the stria vascularis (Chu et al., 2016). If protection requires the drug to enter the inner ear, it is possible that atorvastatin crosses this blood-labyrinth barrier and simvastatin does not (or does so less readily). Other pharmacokinetic differences, including differences in lipophilicity, half-life and potency, may also contribute to differential effects on cisplatin ototoxicity (Zhou and Liao, 2010). Atorvastatin has a longer half-life (20 h versus 12 h) and greater bioavailability (12% versus <5%) relative to simvastatin (Sirtori et al., 2014).

Experimental and clinical studies have shown that statins have a variety of pleiotropic effects. Statins modulate endothelial function (Oikonomou et al., 2014) and reduce inflammation (Jain and Ridker, 2005; Ridker et al., 2005), two effects that may be important in the modulation of cisplatin ototoxicity (see e.g., Shi, 2016). Dysfunction of the stria vascularis due to inflammation of the intra-strial fluid-blood barrier is associated with noise-induced hearing loss (Shi, 2009), age-related hearing loss (Neng et al., 2015), and autoimmune inner ear disease (Lin and Trune, 1997) in animal models. Finally, statins are known inducers of heme oxygenase-1 (Hmox-1, also called HSP32; Lee et al., 2004, Chen et al., 2006, Kwok et al., 2012), an enzyme that mediates heme catabolismhmox-1 induction significantly reduced sensory hair cell death in primary cultures of inner ear tissue from mice (Baker et al., 2015) and lovastatin induced Hmox-1 mRNA and reduced cisplatin ototoxicity in mice (Fernandez et al., 2020). Moreover, in mouse model of noise-induced hearing loss, simvastatin, atorvastatin, lovastatin, fluvastatin and cerivastatin all promoted the elongation of SGN processes that form critical synapses with cochlear inner hair cells (Whitlon et al., 2015).

Large databases of health outcomes (UnitedHealthcare, Specialized Program of Research Excellence (SPORE), SEER-Medicare, Kaiser Permanente Research Bank, Optum), etc. rarely (or never) include audiometric data, and most adults with cancer do not receive a baseline hearing test prior to onset of cisplatin therapy (Konrad-Martin et al., 2018). Therefore, retrospective data from two sites with ongoing ototoxicity monitoring programs was utilized, and a prospective study designed to provide additional subjects for this dataset was initiated (see e.g., Example 1). The combined retrospective and prospective dataset consisted of 277 subjects with head and neck cancer, 40% of whom were taking a statin drug at the onset of cisplatin-based CRT. Significant reductions in cisplatin-induced threshold shifts were observed in the high frequency region (>4 kHz) among statin users, particularly for those on atorvastatin. Similarly, the incidence of a CTCAE-defined hearing loss was significantly reduced by 10% (from 48.8% to 38.4%) for those in the any statin user group and by 18% (from 48.8% to 31.2%) in the atorvastatin user group. Similar results were observed using TUNE criteria. These findings were most prominent among males receiving high cumulative doses of cisplatin (>200 mg/m2) combined with radiation therapy. This observation may be reflective of the study cohort, because head and neck cancer is more common in men than in women and is often treated with these higher cumulative cisplatin doses. Further investigation is necessary in order to fully explore the potential benefit of atorvastatin in female patients, other cancer types, and other cisplatin regimens.

In the current study, the incidence of hearing loss was significantly predicted by three variables: cumulative cisplatin dose, baseline hearing status, and statin use. Accounting for the greatest amount of variance was cumulative cisplatin dose. The median cumulative cisplatin dose in our cohort was 200 mg/m² (IQR: 140-280). Cumulative cisplatin doses higher than 210 mg/m² (Schell et al., 1989, Scobiola et al., 2017) have been previously associated with increased risk for hearing loss (Rademaker-Lakhai et al., 2006). Individuals with normal hearing (PTA≤20 dB HL) at baseline were more likely to experience cisplatin-induced changes in hearing than individuals with hearing loss (PTA >20 dB HL), consistent with previous reports (Flemming et al., 1985) (see e.g., Example 1). As cisplatin ototoxicity is first observed at higher frequencies, which are also the first frequencies affected by noise-induced and age-related hearing loss, it seems plausible that individuals with normal function of the cochlear regions that detect these higher frequencies have more to lose in terms of threshold shifts during cisplatin therapy. As observed in Example 1, the non-statin users in the cohort entered the study with slightly better hearing sensitivity than the atorvastatin users. 54% of non-statin users, compared to 38% of atorvastatin users, had clinically normal hearing (PTA of 1, 2, and 4 kHz≤20 dB HL). However, the majority of non-statin users (88.8%) and atorvastatin users (84%) had either normal hearing or mild hearing loss (PTA of 1, 2, and 4 kHz >20 and <40 dB HL) at baseline (Table 6). Threshold shifts across all users ranged from 0 to 85 dB, and importantly 95% of threshold shifts were ≤50 dB (FIG. 2B), indicating that the differences observed between statin users and non-statin users was not due to a floor effect in calculated threshold shifts.

The third variable that significantly influenced cisplatin-induced hearing loss in the study was the use of atorvastatin. The incidence of hearing loss, per CTCAE criteria, was 31% in atorvastatin users, compared to 49% in those not taking a statin (see e.g., Example 1). Odds ratio estimates indicate that, controlling for overall cumulative cisplatin dose and the presence of a pre-existing hearing loss, an individual taking atorvastatin concurrently with cisplatin therapy is 53% less likely to acquire a CTCAE-defined cisplatin-induced hearing loss compared to a non-statin user. Similar results were obtained using the TUNE ototoxicity criteria. Both CTCAE and TUNE establish criteria for a clinically-meaningful hearing loss that would be expected to impact daily communication and quality of life (Ciorba et al., 2012, NIH National Cancer Institute (2017, www.ctep.cancer.gov)). In addition to an overall loss in hearing sensitivity, a loss of hearing at frequencies above 4 kHz diminishes the ability to recognize and appreciate sounds in nature and music (Theunissen et al., 2014, Prestes et al., 2009). Hearing loss at or below approximately 4 kHz may compromise speech intelligibility in noisy environments. These grading scales help to identify changes in hearing relative to speech communication as well as define the severity of hearing loss. Adverse events that meet or exceed Grade 2 can be dose-limiting (NIH National Cancer Institute (2017, www.ctep.cancer.gov). In the current study, the incidence of a Grade 2 or higher CTCAE hearing loss was significantly reduced by 19.7% in atorvastatin users relative to non-statin users.

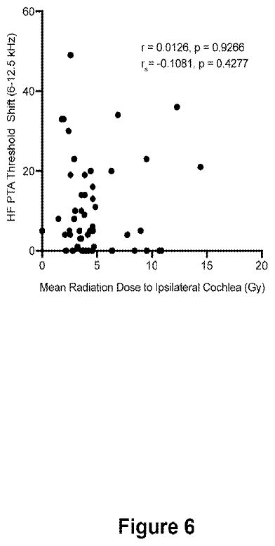

Previous studies have indicated that radiation to the cochlea is independently ototoxic (Bhandare et al., Theunissen et al., 2015). The effects of radiation on hearing loss in the prospective cohort were evaluated. No correlation between cochlear radiation dose and average high-frequency (6 to 12.5 kHz) threshold shifts were observed (see FIG. 6). These data are consistent with prior studies suggesting that hearing loss as a result of radiation alone is uncommon when radiation doses to the cochlea are below 35 Gy (Bhandare et al., Theunissen et al., 2015). With modern intensity-modulated radiotherapy (IMRT) techniques, treatment for tumors of the oropharynx, larynx, hypopharynx, and oral cavity usually results in cochlear radiation doses that are far below this threshold. In the prospective cohort, radiation doses to the cochlea ranged from 0 to 14.4 Gy. Although radiation dose data were not available in the retrospective cohorts, almost all subjects in both non-statin (98.2%) and atorvastatin groups (100%) received radiation, and only 5% of subjects had tumors near the cochlea (e.g., nasopharyngeal carcinoma) that might result in higher cochlear radiation doses (see e.g., Example 1). Thus, it is unlikely that the differences in cisplatin-induced hearing loss that was observed between atorvastatin users and those not taking a statin were influenced by differences in radiation to the cochlea.

Adverse events associated with statin use are rare. Musculoskeletal side effects are the main concern and a common cause of discontinued statin use. Atorvastatin is an attractive option for individuals with statin intolerance, since its longer half-life allows some users to adopt an alternate-day (Adhyaru and Bhavin, 2018) or biweekly (Ghattas and Pimenta, 2007, Christou et al., 2014) dosing schedule. In the study, reduced hearing loss in atorvastatin users was independent of the dose of atorvastatin they were taking (10-80 mg; R²=0.0246). According to the American College of Cardiology (ACC) and American Heart Association (AHA) statin dose guidelines, relative to simvastatin and pravasatin, atorvastatin has a higher dose-potency ratio (Jones et al., 1998, Stone et al., 2014, Adams et al., 2015), thus a lower dose of atorvastatin may be as effective at reducing hyperlipidemia as a higher dose of another statin drug. Furthermore, low-dose atorvastatin may be effective at reducing cisplatin-induced hearing loss.

With any potential drug intended to reduce the toxicities of anti-cancer therapy, there is a concern about introducing a negative impact on survival and other cancer-related outcomes. Importantly, in the study, 3-year survival (see e.g., Example 1) analyses of the largest retrospective cohort suggest that statin drugs, and atorvastatin in particular, did not have a significant effect on patient survival (χ² log-rank=0.09, p>0.05) (Lee et al., 2016, Shen et al., 2018, Ung et al., 2018). Interestingly, numerous prior preclinical and clinical studies suggest that statins do not reduce the therapeutic efficacy of cisplatin, and several epidemiologic studies have reported improved survival among statin users with cancer (Lee et al., 2016, Seckl et al., 2017; Xie et al., 2017; Beckwitt et al., 2018; Jian-Yu et al., 2018, Ung et al., 2018, Hameed et al., 2014; Sutbas et al., 2007).

Two recent large, retrospective studies using the SEER Medicare Database and the Ontario Cancer Registry showed that head and neck cancer patients who were taking statins drugs at the time of diagnosis had improved overall and disease-specific survival (Lebo et al., 2018; Gupta et al., 2019). The study was likely not powered to detect subtle survival differences in a population with a high proportion of oropharyngeal cancers with favorable prognosis. Further, the survival curves included a high proportion of censored data points due to patients lost to follow-up. Additionally, the study did not control for compliance with oral statin medications. In practice, drug compliance is variable, especially for drugs like statins that do not have perceptible effects. It is possible that the beneficial effects of statins on cisplatin ototoxicity are greater than were observed, assuming that some statin users in the cohort skipped doses of statin medications during their cisplatin therapy. Taken together, the data indicates that adding atorvastatin to cisplatin chemoradiation does not reduce the therapeutic efficacy of cisplatin in patients with head and neck cancer.

Furthermore, in the retrospective/observational prospective cohort, 54.7% of all adults treated with cisplatin for head and neck cancer developed a clinically meaningful hearing loss, per CTCAEv5.0 ototoxicity criteria. The observational data show that those on once daily atorvastatin were 45% less likely to develop a cisplatin-related hearing loss relative to those not on atorvastatin (OR 0.55, 95% CI: 0.327-0.942). The study was designed to determine the extent to which 20 mg atorvastatin reduces cisplatin-induced hearing loss.

In some embodiments provided herein is a method of reducing or preventing drug-induced hearing loss in an individual receiving the drug, or intended to receive the drug the method comprising administering to the individual a compound that possesses one or more pharmacological activities of atorvastatin, and/or wherein the compound possesses one or more pharmacokinetic parameters substantially similar to one or more pharmacokinetic parameters of atorvastatin. In some embodiments, the one or more pharmacokinetic parameters is/are selected from the group consisting of the ability to cross a blood barrier, lipophilicity, half-life, potency, bioavailability, absorption, and excretion.

Although all statins share a common mechanism of action, they differ in terms of their chemical structures, pharmacokinetic profiles, and lipid-modifying efficacy (see e.g., Schachter et al., “Chemical, pharmacokinetic and pharmacodynamic properties of statins” Fundam Clin Pharmacol., 19(1): 117-25 (2005). The chemical structures of statins govern their water solubility, which in turn influences their absorption, distribution, metabolism and excretion. Lovastatin, pravastatin and simvastatin are derived from fungal metabolites and have elimination half-lives of 1-3 h. Atorvastatin, cerivastatin (withdrawn from clinical use in 2001), fluvastatin, pitavastatin and rosuvastatin are fully synthetic compounds, with elimination half-lives ranging from 1 h for fluvastatin to 19 h for rosuvastatin. Atorvastatin, simvastatin, lovastatin, fluvastatin, cerivastatin and pitavastatin are relatively lipophilic compounds. Lipophilic statins are more susceptible to metabolism by the cytochrome P(450) system, except for pitavastatin, which undergoes limited metabolism via this pathway. Pravastatin and rosuvastatin are relatively hydrophilic and not significantly metabolized by cytochrome P(450) enzymes. All statins are selective for effect in the liver, largely because of efficient first-pass uptake; passive diffusion through hepatocyte cell membranes is primarily responsible for hepatic uptake of lipophilic statins, while hydrophilic agents are taken up by active carrier-mediated processes. Pravastatin and rosuvastatin show greater hepatoselectivity than lipophilic agents, as well as a reduced potential for uptake by peripheral cells. The bioavailability of the statins differs greatly, from 5% for lovastatin and simvastatin to 60% or greater for cerivastatin and pitavastatin. Clinical studies have demonstrated rosuvastatin to be the most effective for reducing low-density lipoprotein cholesterol, followed by atorvastatin, simvastatin and pravastatin. As a class, statins are generally well tolerated and serious adverse events, including muscle toxicity leading to rhabdomyolysis, are rare. Consideration of the differences between the statins helps to provide a rational basis for their use in clinical practice.

In some embodiments, the blood-barrier separates the inner ear from peripheral blood. The mechanisms by which atorvastatin may affect cisplatin-induced hearing loss are unknown. The primary mechanism by which statins control hyperlipidemia is by inhibition of HMG-COA reductase, the rate-limiting enzyme in the cholesterol synthesis pathway. However, as shown in FIG. 2A, the finding that atorvastatin use was associated with reduced cisplatin-induced hearing loss while simvastatin use was not suggests that the protection observed in atorvastatin users may be independent of atorvastatin's effect on HMG Co-A. One possible explanation for the differences observed between atorvastatin and simvastatin may be differential pharmacokinetics of the two drugs in the inner ear. Like the brain, the inner ear is separated from the peripheral blood flow by a selectively-permeable barrier comprised in part of endothelial cells in the stria vascularis (Chu et al., 2016). If protection requires the drug to enter the inner ear, it is possible that atorvastatin crosses this blood-labyrinth barrier and simvastatin does not (or does so less readily).

In some embodiments, the one or more pharmacological activities is/are selected from the group consisting of inhibition of hydroxymethylglutaryl-CoA (HMG-COA) reductase, modulation of endothelial function, reduction of inflammation, induction of heme oxygenase-1 (Hmox-1, and promotion of elongation of spiral ganglion neurons (SGN)).

In some embodiments of the methods disclosed herein, the compound inhibits hydroxymethylglutaryl-CoA (HMG-COA) reductase.

In some embodiments of the methods disclosed herein, the compound is a statin, or a functional derivative thereof.

In some embodiment of the methods disclosed herein, the statin, or functional derivative thereof, is administered at a normally prescribed dose. The dosage form may include one or more of a 10 mg atorvastatin dosage unit, a 20 mg atorvastatin dosage unit, a 40 mg atorvastatin dosage unit, and an 80 mg atorvastatin dosage unit. The dosage form may be a tablet or capsule, and the method may further include coating the tablet. The oral pharmaceutical dosage may include one or more of a 10 mg atorvastatin dosage unit, a 20 mg atorvastatin dosage unit, a 40 mg atorvastatin dosage unit, and an 80 mg atorvastatin dosage unit.

In some embodiments of the methods disclosed herein, the compound is selected from the group consisting of atorvastatin, fluvastatin, lovastatin, pravastatin, rosuvastatin, simvastatin, pitavastatin, and functional derivatives thereof. Examples of statins with and typical daily adult dose ranges provided in parentheses include: atorvastatin (10-80 mg), fluvastatin (20-80 mg), lovastatin (10-80 mg), pitavastatin (1-4 mg), pravastatin (10-80 mg), rosuvastatin (5-20 mg), and simvastatin (5-80 mg). In the present invention, within the statin user group, six different statins were represented. As shown in Example 1 and Table 6, within the statin user group, six different statins were represented. Of the 113 statin users, atorvastatin (dose range 10-80 mg), was used by 44.2% of subjects (n=50 subjects, 97 ears), simvastatin (dose range 5-40 mg) by 31.9% (36 subjects, 72 ears), pravastatin by 10.6% (12 subjects, 24 ears; dose range 10-80 mg), rosuvastatin by 9.7% (11 subjects, 22 ears; dose range 10-40 mg), lovastatin by 1.8% (2 subjects, 4 ears; 40 mg only) and pitavastatin by 1.8% (2 subjects, 4 ears; 2 mg dose only). Additionally, in FIG. 2A, in subjects not taking a statin (N=324 ears), cisplatin treatment resulted in threshold shifts that were more severe at higher frequencies. Subjects taking any statin (N=219 ears) had significantly less cisplatin-induced hearing loss than subjects who were not taking a statin.

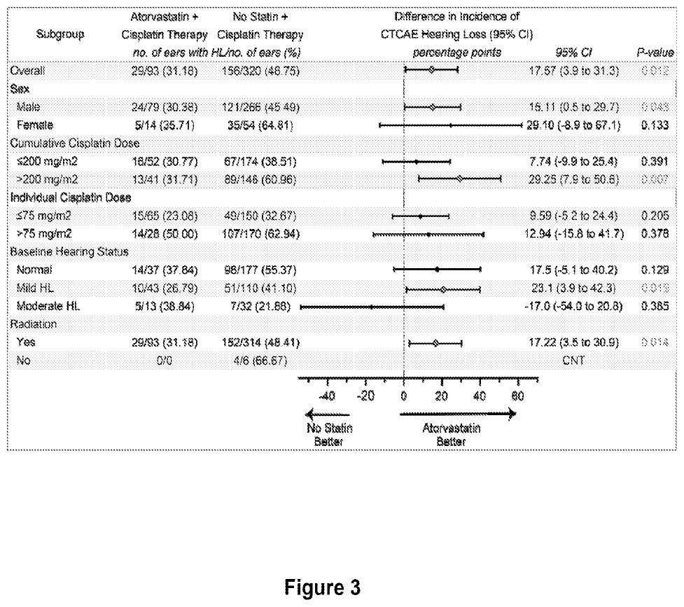

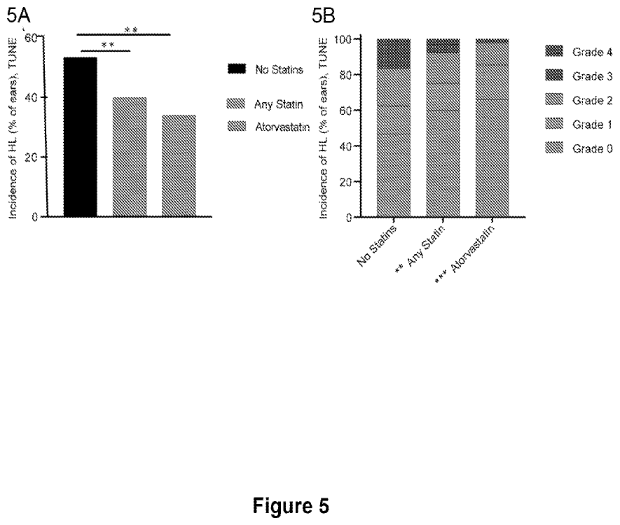

As shown in FIG. 2C, the incidence of cisplatin-induced hearing loss among non-statin users is 48% per CTCAE criteria. Subjects taking any statin had significantly lower incidence of hearing loss than non-statin users. The incidence of hearing loss was further reduced among atorvastatin users. The benefits of atorvastatin to the incidence of a CTCAE-defined hearing loss were generally consistent across all subgroups (FIG. 3). Similar results were obtained when the TUNE (Theunissen et al., 2014) ototoxicity grading criteria was applied (FIG. 5). Together these data indicate that using the CTCAEv5.0 or TUNE grading scale criteria, the incidence of cisplatin-induced hearing loss was reduced in atorvastatin users relative to subjects not taking a statin. In some embodiments of the methods disclosed herein, the compound is atorvastatin, or a functional derivative thereof.

In some embodiments of the methods disclosed herein, the individual has, or is suspected of having, cancer. Furthermore, in some embodiments of the methods disclosed herein, the cancer is a cancer of the head or neck.

Cisplatin is among the most effective and widely-used anti-cancer chemotherapy drugs, used to treat a variety of solid tumors, including testicular, ovarian, bladder, cervical, head and neck, and numerous other malignancies. Due in part to the efficacy of cisplatin (Miller et al., 2019). However, many individuals treated with cisplatin experience significant toxicities, including nephrotoxicity, myelosuppression and ototoxicity. Approximately 60% of adult patients who undergo cisplatin treatment acquire a permanent hearing loss (Frisinia et al., 1984, Bertolini et al., 2004, Coradini et al., 2007, Knight et al., 2017 Marchnitz et al., 2018). Hearing loss is a burdensome side effect for cancer survivors; it compromises daily communication with friends and family and can lead to loneliness, isolation, and frustration (Ciorba et al., 2012). Cisplatin-induced hearing loss is permanent, and there are currently no FDA-approved therapies to prevent or reverse cisplatin ototoxicity. In some embodiments of the methods disclosed herein, the drug is selected from the group consisting of a non-steroidal anti-inflammatory agent, an antibiotic, a chemotherapeutic agent, a diuretic, and a quinine-based compound.

In some embodiments of the methods disclosed herein, the drug is a chemotherapeutic agent. Furthermore, in some embodiments of the methods disclosed herein, the drug possesses one or more activities of cisplatin or carboplatin. Yet further, in some embodiments of the methods disclosed herein, the drug is cisplatin, carboplatin, or a functional derivative thereof.

In some embodiments of the methods disclosed herein, the compound is administered at a time prior to the individual receiving a first administration of the drug. Furthermore, in some embodiments of the methods disclosed herein, the compound is administered during the time period the individual is administered the drug.

In some embodiments of the methods disclosed herein, reducing hearing loss comprises reducing the threshold shift. For example, Example 1 and FIG. 2A demonstrates that atorvastatin use is associated with reduced cisplatin-induced hearing loss. Baseline audiometric thresholds were compared to thresholds obtained following cisplatin treatment to determine threshold shifts. In subjects not taking a statin (N=324 ears), cisplatin treatment resulted in threshold shifts that were more severe at higher frequencies. Subjects taking any statin (N=219 ears) had significantly less cisplatin-induced hearing loss than subjects who were not taking a statin. Threshold shifts at frequencies ≥4 kHz were significantly reduced among subjects taking any statin relative to non-statin users (P<0.02). Atorvastatin users (N=97 ears) had significantly less cisplatin-induced hearing loss than non-statin users (P<0.001).

In another embodiment, a kit for reducing or preventing drug-induced hearing loss in an individual receiving the drug, or intended to receive the drug, the kit comprising: 1) a compound that reduces or prevents the drug-induced hearing loss, wherein the compound possesses one or more pharmacological activities of atorvastatin, and/or wherein the compound comprises one or more pharmacokinetic parameters substantially similar to one or more pharmacokinetic parameters of atorvastatin; and, 2) instructions for administering the compound to the individual.

In some embodiments of the kits disclosed herein, the one or more pharmacokinetic parameters is/are selected from the group consisting of the ability to cross a blood barrier, lipophilicity, half-life, potency, bioavailability, absorption, and excretion. Furthermore, in some embodiments of the kits disclosed herein, the blood-barrier separates the inner ear from peripheral blood.

In some embodiments of the kits disclosed herein, the one or more pharmacological activities is/are selected from the group consisting of inhibition of hydroxymethylglutaryl-CoA (HMG-COA) reductase, modulation of endothelial function, reduction of inflammation, induction of heme oxygenase-1 (Hmox-1, and promotion of elongation of spiral ganglion neurons (SGN)). Furthermore, in some embodiments of the kits disclosed herein, the compound inhibits hydroxymethylglutaryl-CoA (HMG-COA) reductase. Yet further, in some embodiments of the kits disclosed herein, the compound is a statin, or a functional derivative thereof. Yet further, in some embodiments of the kits disclosed herein, the statin, or functional derivative thereof, is administered at a normally prescribed dose.

In some embodiments of the kits disclosed herein, the compound is selected from the group consisting of atorvastatin, fluvastatin, lovastatin, pravastatin, rosuvastatin, simvastatin, pitavastatin, and derivatives thereof. In some embodiment, the compound is atorvastatin, or a functional derivative thereof.

In some embodiments of the kits disclosed herein, the individual has, or is suspected of having, cancer. Furthermore, in some embodiments, the cancer is a cancer of the head or neck.

In some embodiments of the kits disclosed herein, the drug is selected from the group consisting of a non-steroidal anti-inflammatory agent, an antibiotic, a chemotherapeutic agent, a diuretic, and a quinine-based compound. Furthermore, in some embodiments of the kits disclosed herein, the drug is a chemotherapeutic agent.

In some embodiments of the kits disclosed herein, the drug possesses one or more activities of cisplatin or carboplatin. Furthermore, in some embodiments, the drug the drug is cisplatin, carboplatin, or a functional derivative thereof.

In another embodiment, a use of a compound that possesses one or more pharmacological activities of atorvastatin, and/or that possesses one or more pharmacokinetic parameters substantially similar to one or more pharmacokinetic parameters of atorvastatin, in the preparation of a medicament for reducing or preventing drug-induced hearing loss in an individual.

In some embodiments of the uses disclosed herein, the one or more pharmacokinetic parameters is/are selected from the group consisting of the ability to cross a blood barrier, lipophilicity, half-life, potency, bioavailability, absorption, and excretion. Furthermore, in some embodiments of the uses disclosed herein, the blood-barrier separates the inner ear from peripheral blood.

In some embodiments of the uses disclosed herein, the one or more pharmacological activities is/are selected from the group consisting of inhibition of hydroxymethylglutaryl-CoA (HMG-COA) reductase, modulation of endothelial function, reduction of inflammation, induction of heme oxygenase-1 (Hmox-1, and promotion of elongation of spiral ganglion neurons (SGN)). Furthermore, in some embodiments of the uses disclosed herein, the compound inhibits hydroxymethylglutaryl-CoA (HMG-COA) reductase.

In some embodiments of the uses disclosed herein, the compound is a statin, or a functional derivative thereof. Furthermore, in some embodiments of the uses disclosed herein, the compound is selected from the group consisting of atorvastatin, fluvastatin, lovastatin, pravastatin, rosuvastatin, simvastatin, pitavastatin, and functional derivatives thereof.

In some embodiments of the uses disclosed herein, the compound is atorvastatin, or a functional derivative thereof. Furthermore, in some embodiments of the uses disclosed herein, the individual has, or is suspected of having, cancer. In some embodiments, the cancer is a cancer of the head or neck. Yet further, in some embodiments of the uses disclosed herein, the drug is selected from the group consisting of a non-steroidal anti-inflammatory agent, an antibiotic, a chemotherapeutic agent, a diuretic, and a quinine-based compound. In some embodiments, the drug is a chemotherapeutic agent. Yet further, in some embodiments of the uses disclosed herein, the drug possesses one or more activities of cisplatin or carboplatin. In some embodiments, the drug is selected from the group consisting of cisplatin, carboplatin, and functional derivative thereof.

The invention will be further described in the following examples, which do not limit the scope of the invention described in the claims.

### EXAMPLES

The Examples that follow are illustrative of specific embodiments of the invention, and various uses thereof. They are set forth for explanatory purposes only, and are not to be taken as limiting the invention.

### Example 1: Atorvastatin is Associated with Reduced Cisplatin-Induced Hearing Loss

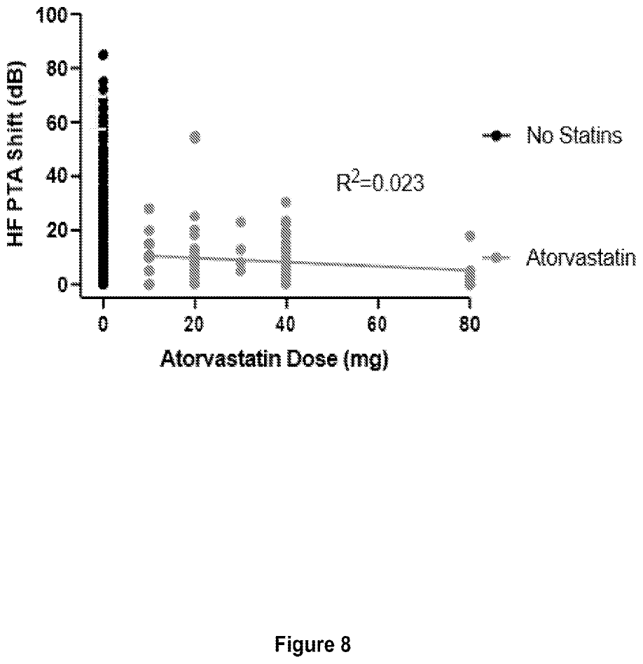

This study consisted of combined retrospective and prospective observational data from three clinical sites (FIG. 8). Subjects included adults, 18 years or older, who were newly diagnosed with head and neck squamous cell carcinoma (HNSCC) and scheduled for treatment with cisplatin. Electronic medical record (EMR) systems were reviewed at each site for subjects meeting full eligibility criteria (Table 1). Characteristics of the subjects (age, sex), details of cancer diagnosis and treatment schedule, and history of statin medication use are shown in Table 2.

TABLE 1
Study Participation Criteria

| Inclusion | Exclusion |
|---|---|
| Adult, 18 yr or older | Prior exposure to cisplatin, taxanes or |
| | cytotoxic chemotherapy drugs |
| Confirmed HNSCC Diagnosis | Profound hearing loss at baseline^{A} |
| Prescribed cisplatin-based | Indication of active middle ear disease^{B} |
| chemotherapy | |

Head and neck squamous cell carcinoma (HNSCC)
^{A}Pure tone average (PTA) at 1, 2, and 4 kHz >95 dB hearing level (HL)
^{B}Active middle ear disease determined by tympanometry and/or bone conduction audiometry

Retrospective clinical data were examined from ototoxicity monitoring programs at the University of Rochester Medical Center (URMC; n=215) and the Walter Reed National Military Medical Center (WRNMMC; n=34). Table 3 describes the type of data contributed by each site. Prospective data were collected as an observational study conducted at the National Institutes of Health (NIH; n=28) in partnership with the Johns Hopkins University (JHU) Department of Otolaryngology-Head and Neck Surgery and Department of Radiation Oncology and Molecular Sciences.

TABLE 2
Demographic and Clinical Characteristics of the Subjects

| | | | | |
|---|---|---|---|---|
| All Subjects | No Statins | Any Statin | Atorvastatin | Simvastatin |

Retrospective and Prospective Subjects

| Characteristics | N = 277 | | N = 164 | | N = 113 | | N = 50 | | N = 36 | |
|---|---|---|---|---|---|---|---|---|---|---|
| Median age (IQR), yr | 60 | (54-66) | 58 | (50-63) | 63 | (58-68) | 63 | (58-67) | 63 | (60-72) |
| Male, No. (%) | 234 | (84.48) | 137 | (83.54) | 97 | (85.84) | 43 | (86.00) | 32 | (88.89) |
| Female, No. (%) | 43 | (15.52) | 27 | (16.46) | 16 | (14.16) | 7 | (14.00) | 4 | (11.11) |
| Median cisplatin | 200 | (140-280) | 200 | (140-280) | 200 | (140-280) | 240 | (160-280) | 200 | (145-280) |
| cumulative dose | | | | | | | | | | |
| (IQR), mg/m² | | | | | | | | | | |
| Radiation, No. (%) | 271 | (98.55) | 159 | (98.15) | 112 | (99.12) | 50 | (100.00) | 35 | (97.22) |

Tumor Site, No. (%)

| | | | | | | | | | | |
|---|---|---|---|---|---|---|---|---|---|---|
| Hypopharynx | 7 | (2.53) | 4 | (2.44) | 3 | (2.65) | 1 | (2.00) | 2 | (5.56) |
| Larynx | 39 | (14.08) | 26 | (15.85) | 13 | (11.50) | 6 | (12.00) | 4 | (11.11) |
| Nasopharynx | 15 | (5.42) | 12 | (7.32) | 3 | (2.65) | 3 | (6.00) | 0 | (0) |
| Oral Cavity | 16 | (5.78) | 9 | (5.49) | 7 | (6.19) | 3 | (6.00) | 2 | (5.56) |
| Oropharynx | 161 | (58.12) | 88 | (53.66) | 73 | (64.60) | 33 | (66.00) | 23 | (63.89) |
| Other^{A} | 39 | (14.07) | 25 | (15.24) | 13 | (12.41) | 4 | (8.00) | 5 | (13.88) |

Site-Specific Contributions
Retrospective Data, No. (%)

| | | | | | | | | | | |
|---|---|---|---|---|---|---|---|---|---|---|
| University of | 215 | (77.62) | 131 | (79.88) | 84 | (74.34) | 36 | (72.00) | 30 | (83.33) |
| Rochester Medical | | | | | | | | | | |
| Center (URMC) | | | | | | | | | | |
| Walter Reed National | 34 | (12.27) | 18 | (10.98) | 16 | (14.16) | 7 | (14.00) | 4 | (11.11) |
| Military Medical | | | | | | | | | | |
| Center (WRNMMC) | | | | | | | | | | |

Prospective Observational Data, No. (%)

| | | | | | | | | | | |
|---|---|---|---|---|---|---|---|---|---|---|
| National Institutes of | 28 | (10.11) | 15 | (9.15) | 13 | (11.50) | 7 | (14.00) | 2 | (5.56) |
| Health/Johns Hopkins | | | | | | | | | | |
| University (NIH/JHU) | | | | | | | | | | |

IQR denotes interquartile range
^{A}Other tumor sites included sinusoidal (3%), cutaneous (1.4%), salivary (1.4%), orbit (<1%), trachea (<1%), and tumors that had an unknown primary site (7.2%)

TABLE 3
Site Contributions

| | Retrospective Data | | Prospective Data |
|---|---|---|---|
| | University of | Walter Reed | National Institutes of |
| | Rochester | National Military | Health (NIH)/Johns |
| | Medical Center | Medical Center | Hopkins University |
| | (URMC) | (WRNMMC) | (JHU) |
| | N = 215 (78%) | N = 34 (12%) | N = 28 (10%) |
| Subject Characteristics (Table 1) | ✓ | ✓ | ✓ |
| Concomittent Statin Medications | ✓ | ✓ | ✓ |
| Cancer Diagnosis/Treatment Parameters | ✓ | ✓ | ✓ |
| Assessments of Middle Ear Function | Bone Conduction | Tympanometry | Tympanometry |
| Auditory Assessments | Std.^{A} + EHF^{B} | SRO^{C} | Std.^{A} + EHF^{B} |

^{A}Standard audiometric frequencies include 1, 2, 3, 4, 6, and 8 kHz
^{B}Extended high frequencies include 10 and 12 or 12.5 kHz
^{C}Sensitive region for ototoxicity frequencies include 0.25, 1, 1.5, 2, 3, 4, 5.6, 6, 6.3, 7.1, 8, 9, 10, 11.2, 12.5 kHz

### Data Analyses

Based on an initial subset of retrospective data from URMC (n=55) and WRNMMC (n=20) showing a 2:1 ratio of non-statin to statin users, a sample size estimate was performed using nQuery (Statsols Solutions Ltd). A sample size of 267 subjects was determined to be necessary to detect the observed 17.3% difference in the incidence of hearing loss as defined by the National Cancer Institute (NCI) Common Terminology Criteria for Adverse Events (CTCAE), between statin users and non-statin users, with an alpha of 0.05 and 80% power. These initial 75 subjects were included in the final analysis totaling 277 subjects.

The primary outcome measure was the difference in incidence of a change in hearing between the baseline hearing test and the post-treatment hearing test, per ear, as defined by established ototoxicity grading scales. These scales report subjects who transition from normal hearing to hearing loss as well as those who transition from some hearing loss to more hearing loss. The criteria of two different grading scales was applied to the dataset (Table 4). The NCI CTCAEv5.0(34) classifies adverse changes in hearing based on auditory threshold shifts across a 1-8 KHz frequency range. TUNE ototoxicity grading criteria (Theunissen et al., 2014), which incorporate extended high frequency (EHF) data was also applied (see Table 5, FIG. 5).

TABLE 4
Ototoxicity Grading Criteria^{A}

| | Frequency | | | |
|---|---|---|---|---|
| Scale | Range | Grade | Criteria | Reference |
| CTCAE | 1-8 kHz | Grade 1 | Average 15-25 dB TS at 2 consecutive frequencies | National Cancer |
| | | Grade 2 | Average >25 dB TS at 2 consecutive frequencies | Institute |
| | | Grade 3 | Average >25 dB TS at 3 consecutive frequencies | (NCI), 2017 |
| | | Grade 4 | Absolute threshold >80 dB at 2 kHz and above | |
| TUNE | PTA 1-2-4 or | Grade 1 | ≥10 dB TS at 1-2-4 kHz or 6-8-12 kHz | Theunissen |
| | 8-10-12 kHz | Grade 2 | ≥20 dB TS at 1-2-4 kHz or 6-8-12 kHz | et al., 2014 |
| | | Grade 3^{B} | ≥35 dB TS at 1-2-4 kHz or 6-8-12 kHz | |
| | | Grade 4^{B} | ≥50 dB TS at 1-2-4 kHz or 6-8-12 kHz | |

^{A}Data based on shifts in dB hearing levels obtained using air conduction (AC) audiometry
^{B}Scale criteria modified from original reference to accommodate threshold shift data.

TABLE 5
Logistic Regression with Adjusted Odds Ratios (OR)
on the incidence of a CTCAE or TUNE Hearing Loss^{A}

| CTCAE | | | | | TUNE | | | |
|---|---|---|---|---|---|---|---|---|
| Effect | X² | df | P value | OR (95% CI) | X² | df | P value | OR (95% CI) |
| Atorvastatin Use | 7.46 | 1 | 0.006 | 0.48 (0.29-0.81) | 9.35 | 1 | 0.002 | 0.46 (0.28-0.76) |
| Cisplatin Dose^{B} | 25.34 | 1 | <0.001 | 1.01 (1.01-1.01) | 14.77 | 1 | <0.001 | 1.01 (1.00-1.01) |
| Baseline Hearing^{C} | 9.55 | 1 | 0.002 | 0.60 (0.44-0.83) | 13.38 | 1 | <0.001 | 0.56 (0.41-0.76) |

Confidence intervals (CI)
^{A}Hearing loss defined as a change in hearing meeting CTCAE or TUNE Grade 1 minimum criteria.
^{B}Cisplatin dose is cumulative cisplatin dose over length of cisplatin therapy. OR data calculated based on units of 100 mg/m².
^{C}Baseline hearing based on the pure tone average (PTA) of 1, 2, and 4 kHz.

The secondary outcome measure was the change in hearing thresholds, per ear, between the baseline hearing test and the post-treatment hearing test across standard audiometric and EHFs ranging from 0.25 to 12.5 kHz. All baseline audiograms were obtained ≤90 days prior to start of cisplatin treatment, and post-treatment audiograms were obtained≤90 days following the end of cisplatin treatment. Threshold shifts were calculated as the difference in threshold (dB HL) between baseline and post-treatment audiograms at each frequency.

### Statistics

Subjects were initially assigned to either the non-statin user group or the statin user group based on their use of any statin medication at the start of cisplatin treatment. Based on the prevalence of each statin type within the statin-user group (see Table 6), atorvastatin and simvastatin in isolation were also examined. Both ears were used in the data analyses due to possible influences of asymmetric hearing losses either at baseline (observed in 12% of all subjects, defined as a ≥15 dB difference in the PTA of 0.5, 1, 2 and 4 kHz) and/or a result of differential radiation doses to the two cochleas, since high doses of radiation (>45 Gy) have been reported to be ototoxic (Bhandare et al., 2010). Including both ears from each subject introduces correlation among observations that can incorrectly reduce the standard error of certain estimates. Subject-specific random intercepts in the statistical model (Fitzmaurice et al., 2012) was included to correct for bias. This method allows us to make use of all of the data from each ear while accounting for the fact that the two ears of each individual will be correlated.

A 2-way analysis of variance (ANOVA) using Dunnett post hoc multiple comparisons (Prism v8, GraphPad Software) was conducted to compare the threshold shifts as a function of frequency (0.25 to 12.5 kHz) between non-statin users and subjects taking any statin, atorvastatin, or simvastatin. However, while the comparison between atorvastatin and non-statin users remained sufficiently powered (>0.8).

The primary outcome measure was based on changes in hearing as defined by CTCAEv5.0 scale criteria (see Table 4) and were analyzed using categorical incidence (per ear) data. The incidence and severity distribution of a clinically meaningful hearing change, per ear, relative to statin use was analyzed using chi-square analyses (SAS PROC FREQ procedure). The rate difference, with 95% confidence intervals, of a CTCAE-defined hearing loss between atorvastatin and non-statin users was estimated by fitting the Poisson model using PROC NLMIXEDA for the total population as well as for subgroups (sex, cumulative cisplatin dose, individual cisplatin dose, baseline hearing status and radiation). A logistic regression analysis (SAS PROC LOGISTIC procedure) with calculation of odds ratios and 95% confidence intervals was performed to identify associations between CTCAE-defined changes in hearing and statin use after adjustment for significant covariates.

The secondary outcome measure utilized high frequency audiometric threshold data. A mixed-effect model analysis (SAS PROC GLIMMIX procedure) was applied to average high frequency threshold shift data (pure tone average (PTA) of 6-12.5 kHz) to determine the influence of other model effects on cisplatin-induced threshold shift within his high frequency region. Fixed effects included statin use, sex, age, cumulative cisplatin dose, radiation exposure and baseline hearing. Subject ID was defined as a random effect to account for the inclusion of two ears in the analysis. Statin use, sex, and radiation exposure were included as dichotomous variables, whereas age, cumulative dose, and baseline hearing based on the PTA of 1, 2, and 4 kHz were treated as continuous variables. A Pearson R correlation analysis was used to assess the association of atorvastatin drug dose and high frequency threshold shift within Prism v8.

Overall survival and disease-free survival were calculated as Kaplan-Meier curves using Prism v8 software on all available data from URMC at up to 3 years post treatment (N=175). Survival curves were compared using the log-rank (Mantel-Cox) test. Cochlear radiation dose data presented in FIG. 6 were analyzed using Pearson R and Spearman R correlation analysis within Prism v8.

An alpha of 0.05 was set for all analyses. All statistical analyses were performed in SAS, version 9.4 (SAS Institute Inc.) or Prism v8.

Inclusion criteria

All subjects were 18 years or older, newly diagnosed with head and neck squamous cell carcinoma (HNSCC), and scheduled for treatment with cisplatin.

### Exclusion Criteria

Excluded subjects those who had previously received cisplatin, taxanes or other cytotoxic chemotherapy drugs, those whose baseline audiogram was >90 days before onset of first cisplatin treatment, those who at the time of their baseline hearing evaluation, auditory thresholds exceeded a pure tone average (PTA) of 95 dB HL average at 1-2-4 kHz or there was indication of active middle ear disease, and those whose follow-up audiogram was >90 days after cessation of last cisplatin treatment.

### Site-Specific Contributions and Study Design

This study consisted of combined retrospective and prospective observational data obtained from three clinical sites. Audiometric data collected≤90 days from the onset of cisplatin therapy were compared against audiometric data collected≤90 days from completion of cisplatin therapy to determine threshold shifts. Subjects whose baseline audiogram was collected up to 1 week after the first cisplatin infusion (n=12 subjects) were included only if their hearing was within normal limits (≤20 dB HL from 1 to 8 kHz).

Retrospective data were collected from the University of Rochester Medical Center (URMC) (N=215 subjects). All audiometric data were collected in a sound-attenuated booth using either a Grason Stadler GSI 61 or SGI AudioStar Pro audiometer and Telephonics TDH50 headphones or EAR ER3A insert earphones and Sennheiser HDA 200 headphones. Air conduction (AC) thresholds for standard frequencies (0.25 to 8 kHz) as well as 12 kHz were obtained. Bone conduction (BC) audiometric thresholds were reported for 1, 2, and 4 kHz and used to screen for the presence of active middle ear disease. Additional retrospective data were collected from Walter Reed National Military Medical Center (WRNMMC) (N=34 subjects). AC thresholds were collected in a sound-attenuated booth for standard audiometric frequencies (0.25 to 8 kHz) as well as over the sensitive range for ototoxicity (SRO), up to 12.5 kHz, using an Otometrics Madsen Astera audiometer and with Sennheiser HDA-200 or RadioEar IP30 headphones. Tympanometry was used to screen for active middle ear disease. Prior to data sharing with NIH collaborators for analyses, URMC and WRNMMC removed personal identifiable information (PII)/personal health information (PHI) from the dataset. Coded IDs were assigned to each subject, and the code was not shared with NIH investigators.

Prospective data were collected in a collaborative, observational study through a National Institutes of Health (NIH) partnership with Johns Hopkins University (JHU). Audiometric data were collected using an FDA-approved SHOEBOX iPad-based audiometer (Clearwater Clinical, Inc), with Sennheiser HDA-280 headphones (ANSI S3.6) (National Cancer Institute (NCI). Common Terminology Criteria for Adverse Events (CTCAE)), for standard test frequencies (1 to 8 kHz) and extended high frequencies (EHF) including 10 and 12.5 kHz. The SHOEBOX Audiometer has been validated for use outside of a sound booth (Brock et al., 1991). All auditory thresholds were measured in a quiet meeting room with SHOEBOX Smart Testing enabled to monitor ambient noise. Tympanometry (MT10 Interacoustics) was used to screen for the presence of active middle ear disease. AC thresholds for all 277 subjects were analyzed at 0.25, 0.5, 1, 2, 3, 4, 6, 8 and 12.5 kHz from baseline and post-treatment audiograms. Data from URMC collected at 12 kHz were grouped with 12.5 kHz data from WRNMMC and NIH/JHU. Frequencies at which data were available for <70% of the total number of subjects were excluded from analyses; an example of this is the interoctave frequencies measured using SRO monitoring at WRNMMC only. If a subject had no response at the output limits of the audiometer, a threshold value was assigned as the maximum output level plus 5 dB.

### Results

### Characteristics of the Dataset

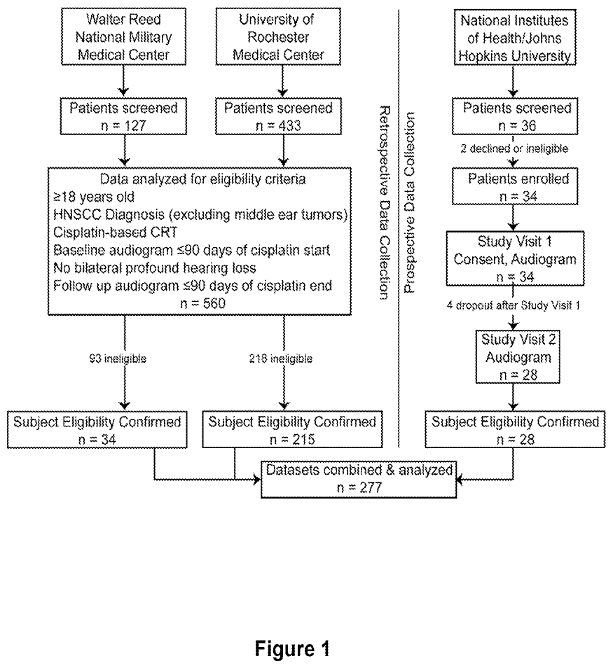

As shown in FIG. 1, retrospective and prospective data were combined for a total of 277 subjects. All subjects were adults newly diagnosed with HNSCC and treated with cisplatin-based CRT. All subjects also met eligibility criteria and the characteristics of the 277 subjects are shown in Table 2. Individuals with middle ear tumors were excluded at screening (n=2) due to active middle ear disease, confirmed by bone conduction audiometry. Of the eligible 277 subjects, six had a unilateral hearing loss at baseline that met exclusion criteria for that ear (≥95 dB HL average threshold at 1, 2, and 4 kHz, or an indication of active middle ear disease). These six subjects with unilateral profound hearing loss were retained in the study; however, only the better ear was included in analyses. In total, 546 ears from 277 subjects were included in the analyses. Ears were treated independently in the data analysis due to ear-specific differences in baseline hearing sensitivity and differences in radiation doses to the cochlea. The use of two ears in the dataset was controlled for statistically in a mixed-effect model analysis as a random effect.

### Statin Use Among Study Subjects

Subjects were assigned to groups based on whether they were (or were not) taking a statin medication at the time of initiation of cisplatin therapy. Details pertaining to the duration of statin use prior to baseline data collection and the primary indication necessitating statin use were not obtained. 59.2% (27F, 137M) of subjects were non-statin users, and 40.8 (16F, 97M) were statin users. Table 6 shows that within the statin user group, six different statins were represented. Of the 113 statin users, atorvastatin (dose range 10-80 mg), was used by 44.2% of subjects (n=50 subjects, 97 ears), simvastatin (dose range 5-40 mg) by 31.9% (36 subjects, 72 ears), pravastatin by 10.6% (12 subjects, 24 ears; dose range 10-80 mg), rosuvastatin by 9.7% (11 subjects, 22 ears; dose range 10-40 mg), lovastatin by 1.8% (2 subjects, 4 ears; 40 mg only) and pitavastatin by 1.8% (2 subjects, 4 ears; 2 mg dose only). Cisplatin-induced hearing loss between non-statin users vs. those taking any statin was compared first. Because atorvastatin and simvastatin were highly represented among 113 statin users (76.1% of total), hearing loss between non-statin users vs. those taking atorvastatin (n=50) or simvastatin (n=36) was also compared; however, while the comparison between atorvastatin and non-statin users remained sufficiently powered, the study was underpowered for the comparison of simvastatin users vs. those not taking a statin.

TABLE 6
Clinical Hearing Status^{A} at Pre-and Post-Cisplatin Therapy

| | | | | | | | |
|---|---|---|---|---|---|---|---|
| No Statins | Any Statin | Atorvastatin | Simvastatin | Pravastatin | Rosuvastatin | Pitavastatin | Lovastatin |
| N = 164 | N = 113 | N = 50 | N = 36 | N = 12 | N = 11 | N = 2 | N = 2 |

Hearing Status at Baseline, No. Ears (%)

| Normal | 178 | (54.27) | 98 | (43.36) | 38 | (38.00) | 34 | (47.22) | 15 | (62.50) | 9 | (40.91) | 0 | | 2 | (50.00) |
|---|---|---|---|---|---|---|---|---|---|---|---|---|---|---|---|---|
| Mild | 113 | (34.45) | 95 | (42.04) | 46 | (46.00) | 24 | (33.33) | 6 | (25.00) | 13 | (59.09) | 4 | (100.00) | 2 | (50.00) |

| | | | | | | | | | | | | | |
|---|---|---|---|---|---|---|---|---|---|---|---|---|---|
| Moderate | 32 | (9.76) | 29 | (12.83) | 13 | (13.00) | 13 | (12.50) | 3 | (12.50) | 0 | 0 | 0 |

| | | | | | | | | | | | |
|---|---|---|---|---|---|---|---|---|---|---|---|
| Severe | 2 | (0.61) | 1 | (0.44) | 0 | 1 | (1.39) | 0 | 0 | 0 | 0 |

| | | | | | | | | | | | |
|---|---|---|---|---|---|---|---|---|---|---|---|
| Profound^{B} | 3 | (0.91) | 3 | (1.33) | 3 | (3.00) | 0 | 0 | 0 | 0 | 0 |

Hearing Status at Follow-up, No. Ears (%)

| Normal | 138 | (41.38) | 63 | (28.38) | 26 | (26.80) | 27 | (38.57) | 9 | (37.50) | 2 | (9.09) | 0 | | 0 | |
|---|---|---|---|---|---|---|---|---|---|---|---|---|---|---|---|---|
| Mild | 110 | (33.33) | 95 | (42.79) | 49 | (50.52) | 17 | (24.29) | 8 | (33.33) | 14 | (63.64) | 4 | (100.00) | 4 | (100.00) |

| | | | | | | | | | | | | | | |
|---|---|---|---|---|---|---|---|---|---|---|---|---|---|---|
| Moderate | 72 | (21.82) | 58 | (26.13) | 21 | (21.65) | 25 | (35.71) | 6 | (25.00) | 6 | (27.27) | 0 | 0 |

| | | | | | | | | | | | | |
|---|---|---|---|---|---|---|---|---|---|---|---|---|
| Severe | 6 | (1.82) | 2 | (0.90) | 1 | (1.03) | 0 | 1 | (4.17) | 0 | 0 | 0 |

| | | | | | | | | | | | | |
|---|---|---|---|---|---|---|---|---|---|---|---|---|
| Profound | 4 | (1.21) | 4 | (1.80) | 3 | (3.00) | 1 | (1.43) | 0 | 0 | 0 | 0 |

^{A}Hearing status based on Pure Tone Average (PTA) of 1, 2, and 4 kHz expressed in dB
HL: Normal (PTA ≤20), Mild (PTA >20, <40), Moderate (PTA ≥40, ≤70), Severe (PTA >70, <95), Profound (PTA ≥95)
^{B}Ears with profound hearing loss at baseline were excluded from subsequent analyses

### Tumor Types and Cisplatin Regimens

Most of the subjects had head and neck squamous cell carcinomas (HNSCC) that localized to either the oropharynx (58.1%) or the larynx (14.1%) (Table 2). A small portion of tumors localized to the oral cavity (5.8%), nasopharynx (5.4%) or the hypopharynx (2.5%). Tumors sites classified as “other” comprised 14.1% of all tumor types and consisted of sinonasal (2.9%), cutaneous (1.4%), salivary (1.4%), middle ear (<1%), orbit (<1%), trachea (<1%), and tumors that had an unknown primary site (7.2%). All subjects were treated with cisplatin-based chemotherapy and nearly all (98.6%) had concurrent radiation therapy (CRT). 53.6% of subjects received low-dose, weekly cisplatin treatment, defined as cisplatin doses <75 mg/m² per infusion. The remaining 46.4% of subjects received high-dose cisplatin (>75 mg/m² per infusion) administered approximately every 3 weeks; however, 9.4% of individuals scheduled to receive high-dose therapy were switched to low-dose therapy due to drug intolerance and/or ototoxicity. The median cumulative cisplatin dose for all subjects was 200.9 mg/m² (Interquartile Rang (IQR): 140-280 mg/m². This cumulative dose was consistent across all groups with a cumulative cisplatin dose of 200 mg/m² (IQR: 155-280 mg/m²) for non-statin users, 200 mg/m² (IQR: 135-280 mg/m²) for all statin users, and 240 mg/m² (IQR: 160-280 mg/m²) and 200 mg/m² (IQR: 145-280 mg/m²) for atorvastatin and simvastatin users, respectively.

### Other Subject Characteristics

Other demographic characteristics, such as sex, age and pre-existing hearing loss were comparable across groups (Tables 2 and 6). The median age for all subjects was 60 years (IQR: 54-66). Non-statins users, on average, were slightly younger than those in the any statin user group (median age 58 vs. 63) and had better hearing at baseline (Table 6). Consistent across all groups was a predominance towards males (>83% male) who received concurrent radiation (>97% with radiation). Fifty-four percent of non-statin user ears (n=328) had normal hearing at baseline (defined as the pure tone average (PTA)≤20 dB HL), 34% had a mild hearing loss (PTA>20, <40), and 10% had moderate hearing loss (PTA ≥40, ≤70). Statin user ears (n=226 ears) consisted of 43% normal, 42% mild hearing loss, and 13% moderate hearing loss. Similarly, atorvastatin user ears (n=100 ears) included 38% normal hearing, 46% mild hearing loss, and 13% moderate hearing loss. Following cisplatin therapy, with the exception of those subjects on concurrent pitavastatin (n=2) where both subjects started and ended therapy with a bilateral mild hearing loss, ears analyzed as part of the non-statin user and other five statin user groups showed a decrease in the percentage of ears categorized as having normal hearing and an increase towards more severe hearing loss (Table 6).

Atorvastatin Users have Less Cisplatin-Induced Hearing Loss than Those not Taking a Statin

In total, 546 ears from 277 subjects were included in the analyses. The primary outcome measure is the incidence of CTCAE-defined change in hearing, which relies first on the calculation of the change in auditory thresholds (“threshold shifts”) between the baseline and post-treatment audiograms. Therefore, the analysis was carried out by examining threshold shift data among subjects as a function of their status of concurrent statin use at baseline. On average, cisplatin therapy resulted in a 13.7 dB+18.6 high-frequency threshold shift (PTA of 6, 8, and 12.5 kHz) (FIG. 12A). A two-way ANOVA indicated a significant effect of treatment group (non-statin vs. any statin, atorvastatin, or simvastatin) on threshold shifts (two-way ANOVA, F(_{3, 5802})=29.06, P<0.001) as well as a significant effect of frequency (F(_{8, 5802})=55.87, P<0.001). To explore the significant interaction of the two effects (F(_{24, 5802})=1.599, P<0.001) a Dunnett's multiple comparisons post hoc analysis was conducted comparing threshold shift at each frequency for non-statin users to either the any statin, atorvastatin, or simvastatin user groups. Threshold shifts at frequencies ≥4 kHz were significantly reduced among subjects taking any statin relative to non-statin users (P<0.02). Interestingly, threshold shifts were further reduced among atorvastatin users (P<0.001). Interestingly, in contrast, no significant reduction in threshold shift was observed among simvastatin users. However, while the comparison between atorvastatin and non-statin users remained sufficiently powered (>80% power, alpha=0.05).

Atorvastatin was the most commonly used statin in the cohort and doses ranged from 10 to 80 mg and the relationship between high-frequency hearing loss (PTA of 6, 8, and 12.5 kHz) and atorvastatin dose was examined. As shown in FIG. 2B, no significant correlation (R²=0.023, P>0.05) was found, suggesting that the reduction in the observed hearing loss among atorvastatin users was independent of atorvastatin dose.

A mixed-effect model (MEM) analysis was applied to the average high frequency threshold shift data to examine the contributions of other variables to cisplatin-induced hearing loss. Controlling for all other fixed effects in the model (sex, age, cumulative cisplatin dose, concurrent radiation and pre-existing hearing loss at baseline) and controlling for the inclusion of data from two ears for most subjects (random effect), atorvastatin use was significantly correlated with reduced hearing loss (GLIMMX, F1,204=6.42, P=0.02) (Table 7). Significant effects were also observed for cumulative cisplatin dose (GLIMMX, F1, 204=13.45, P<0.001) and baseline hearing (GLIMMX, F1, 204=17.84, P<0.001). Together, these data indicate that atorvastatin users had significantly less cisplatin-induced hearing loss than non-statin users.

TABLE 7
Mixed-effect Model of High Frequency (6-12 kHz) Hearing Loss^{A}

| | No Statins vs Any Statin | | | No Statins vs Atorvastatin | | | No Statins vs Simvastatin | | |
|---|---|---|---|---|---|---|---|---|---|
| Effect | F value | df | P value | F value | df | P value | F value | df | P value |
| Statin Use | 3.10 | 1,264 | 0.08 | 6.17 | 1,204 | 0.01 | 0.00 | 1,192 | 0.98 |
| Sex | 0.74 | 1,264 | 0.39 | 2.79 | 1,204 | 0.10 | 1.04 | 1,192 | 0.31 |
| Age | 0.37 | 1,264 | 0.55 | 0.36 | 1,204 | 0.55 | 0.36 | 1,192 | 0.55 |
| Cisplatin Dose | 17.01 | 1,264 | <0.001 | 13.36 | 1,204 | <0.001 | 12.98 | 1,192 | <0.001 |
| Radiation | 0.006 | 1,264 | 0.81 | 0.12 | 1,204 | 0.73 | 0.15 | 1,192 | 0.70 |
| Baseline Hearing^{B} | 35.13 | 1,264 | <0.001 | 20.55 | 1,204 | <0.001 | 22.17 | 1,192 | <0.001 |

^{A}High frequency hearing loss is based on the dB HL pure tone average of 6, 8, and 12.5 kHz
^{B}Baseline hearing based on the pure tone average (PTA) of 1, 2, and 4 kHz

Atorvastatin Use is Associated with Reduced Incidence and Severity of Cisplatin-Induced Hearing Loss

CTCAEv5.0(34) criteria was applied next to report the incidence and severity of hearing loss. Among subjects not taking any statin, the incidence of hearing loss was 48.8% (CTCAE, FIG. 2C). The incidence of a Grade 1 or higher hearing loss was significantly reduced from 48.8% in non-statin users to 38.4% (Chi Square, χ²=5.6, P<0.02) in statin users, with further reduction to 31.2% (χ²=9.0, P<0.01) among atorvastatin users. Similar results were obtained when the TUNE (Theunissen et al., 2014) ototoxicity grading criteria was applied (FIG. 5). These data indicate that the incidence of cisplatin-induced hearing loss was significantly reduced in atorvastatin users relative to those not taking a statin.

In addition to incidence, CTCAE reports severity of hearing loss using a 1-4 scale in which Grade 4 denotes the most severe change in hearing. Grade 1 is considered mild where intervention may not be required, while Grade 2 is considered a moderate adverse event for which intervention is indicated (NCI Common Terminology Criteria for Adverse Events (CTCAE)). As shown in FIG. 2D, the incidence of a CTCAE grade ≥2 hearing change was significantly reduced from 29.4% in the non-statin user group to 9.7% for atorvastatin users (Chi Square, χ²=14.9, P=0.0001). These data indicate that cisplatin-induced hearing loss was also less severe among atorvastatin users compared to non-statin users.

As shown in FIG. 3, the benefits of atorvastatin to the incidence of a CTCAE-defined hearing loss were generally consistent across all subgroups. Overall, 48.8% (156 of 320 ears) of ears among non-statin users showed hearing loss, compared to 31.2% (29 of 93 ears) of ears among atorvastatin users. In addition to a significant benefit of atorvastatin use overall, significant benefits in favor of atorvastatin users versus non-statin users were noted among males (30.4% vs. 45.5%), those with higher cumulative cisplatin doses (>200 mg/m²) (31.7% vs. 61%), those with a mild hearing loss at baseline (26.8% vs. 41.1%), and those who underwent chemoradiation therapy (31.2% vs 47.8%).

In addition to the MEM analysis of high-frequency PTA (a continuous variable), logistic regression analysis of the incidence of a CTCAE-defined hearing loss (a binary variable) was also. The logistic regression allowed us to calculate adjusted OR for the three variables identified in the MEM analysis (Table 3) that were significantly associated with cisplatin-induced hearing loss: statin use, cumulative cisplatin dose, and baseline hearing status. Results indicate that for every 100 mg/m² increase in cisplatin dose, an individual is 2.2 times more likely to develop hearing loss (OR=2.20, 95% CI: 1.63-3.01) (Table 5). Additionally, with every 20 dB increase in PTA threshold at baseline, a person is 40% (OR=0.60, 95% CI: 0.44-0.83) less likely to acquire a cisplatin-induced hearing loss. Finally, an individual taking atorvastatin is 53% less likely (OR=0.47, 95% CI: 0.28-0.77) to acquire a cisplatin-induced hearing loss compared to a non-statin user after controlling for cumulative cisplatin dose and baseline hearing status.

### TUNE Analysis

Changes in hearing were primarily defined using CTCAEv5.0 criteria (Theunissen et al., 2014). However, cisplatin-induced ototoxicity is characterized initially as a high frequency (above 8 kHz) hearing loss that can spread to include lower frequencies (Brock et al., 1991). Therefore, the TUNE grading scale (Theunissen et al., 2014) was applied, which reports incidence and severity of hearing loss based on shifts in auditory thresholds across two frequency ranges: 1-4 kHz and 8-12.5 kHz (Table 4). The higher-frequency range of the TUNE scale was modified to include 6, 8 and 12.5 kHz due to insufficient data at 10 kHz in the dataset. Because many of the subjects had some hearing loss at baseline, the TUNE criteria was further modified so that Grades 3 and 4 utilized threshold shift data instead of absolute thresholds. A TUNE Grade 3 was redefined for this study as a ≥35 dB PTA threshold shift from the baseline to the post-treatment audiogram, and similarly Grade 4 was redefined as a ≥50 dB PTA threshold shift.

Changes in hearing, defined by TUNE criteria, were analyzed using categorical incidence (per ear) data. The incidence and severity distribution of a clinically meaningful hearing change, per ear, relative to statin use was analyzed using chi-square analyses (SAS PROC FREQ procedure). The rate difference, with 95% confidence intervals, of a TUNE-defined hearing loss between atorvastatin and non-statin users was estimated by fitting the Poisson model using PROC NLMIXEDA for the total population as well as for subgroups (sex, cumulative cisplatin dose, individual cisplatin dose, baseline hearing status and radiation). A logistic regression analysis (SAS PROC LOGISTIC procedure) with calculation of odds ratios and 95% confidence intervals was performed to identify associations between TUNE-defined changes in hearing and statin use after adjustment for significant covariates.

Among subjects not taking any statin, the incidence of a hearing loss per TUNE criteria was 53.4% (FIG. 5A). The incidence of Grade 1 or higher cisplatin-induced hearing loss was significantly reduced relative to the non-statin user group from 53.4% to 39.9% (χ²2=9.6, p<0.01) in the any-statin user group and 34.0% in the atorvastatin user group, (χ²2=11.2, p<0.001) (FIG. 5A). 36.5% of subjects in the no statin group had a grade 2 or higher change in hearing compared to 14.4% of those in the atorvastatin group (χ²2=21.2, p<0.001) (FIG. 5B).

The logistic regression allowed us to calculate adjusted odds ratios (OR) with 95% confidence intervals for the three variables identified in our mixed effects analysis (MEM) analysis (Table 3) that were significantly associated with cisplatin-induced hearing loss: statin use, cumulative cisplatin dose and baseline hearing status. Using TUNE-defined hearing loss criteria, results indicate that for every 100 mg/m² increase in cisplatin dose, a person is 1.8 times more likely to develop hearing loss (OR=1.80, 95% CI:1.36-2.43). Additionally, with every 20 dB increase in PTA threshold at baseline, a person is 44% (OR=0.56, 95% CI: 0.41-0.76) less likely to acquire a cisplatin-induced hearing loss. Finally, an individual on atorvastatin is 56% less likely (OR=0.44, 95% CI: 0.27-0.72) to acquire a cisplatin-induced hearing loss compared to a non-statin user after controlling for cumulative cisplatin dose and baseline hearing.

### 3-Year Overall Survival and Disease-Free Survival do not Differ Between Atorvastatin Users and Those not Taking a Statin

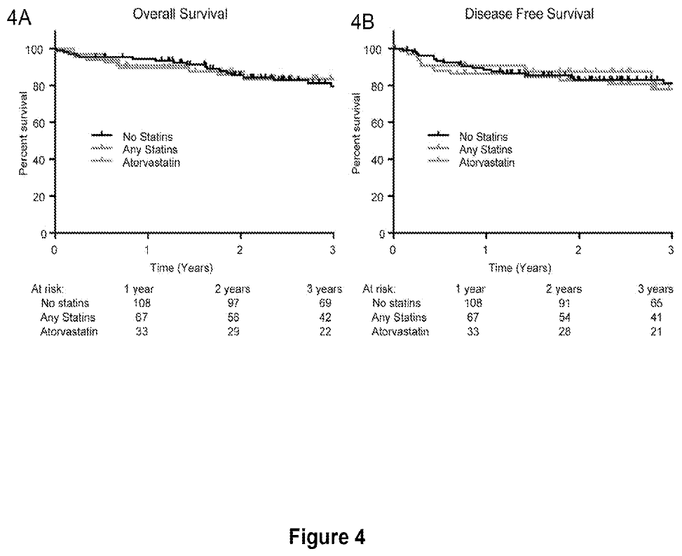

To determine whether statin drugs may impact treatment outcomes in patients with HNSCC the overall survival and disease-free survival was examined on the available data from URMC (n=175) (see e.g., Example 1 showing 3-year survival for atorvastatin users). Overall survival at 3 years was approximately 80% (FIG. 4). An exact median survival time could not be calculated due to the number of patients censored/lost to follow-up. There were no significant differences among non-statin users, statin users, and atorvastatin users in overall (P=0.97) or disease-free survival (p=0.94).

The statin users and non-statin users differed slightly in their ages and anatomic tumor sites, with statin users tending to be older, less likely to have normal hearing at baseline, and more likely to have oropharyngeal cancer. The statin users and non-statin users differed slightly in their age and anatomic tumor site, with statin users tending to be older and more likely to have oropharyngeal cancer. These factors may have contributed slightly to hearing and survival outcomes.

Furthermore, the data indicate that cisplatin therapy results in clinically meaningful (using TUNE criteria) hearing loss in up to 53% of individuals with head and neck cancer. A reduced incidence and severity of cisplatin-induced hearing loss in subjects taking atorvastatin relative to those not taking a statin drug was observed. The data suggest that atorvastatin, an inexpensive drug with a good safety profile, holds promise to reduce cisplatin-induced hearing loss in adult patients without reducing the therapeutic efficacy of cisplatin.

A randomized, placebo-controlled interventional study is currently being developed to determine the extent to which atorvastatin reduces cisplatin-induced hearing loss in patients with head and neck cancer. Subjects with newly-diagnosed head and neck cancer who are scheduled to receive cisplatin-based CRT and are not already taking a statin will be randomized to receive either atorvastatin (20 mg) or placebo daily for the duration of CRT. Baseline hearing sensitivity will be measured prior to onset of cisplatin-therapy and again after completion of all cycles of cisplatin therapy. The primary endpoint is the change in hearing sensitivity between pre- and post-treatment audiograms defined using CTCAE ototoxicity scale criteria. In addition, studies in animal models are needed in order to examine the cellular and molecular mechanisms by which statins may reduce cisplatin-induced hearing loss.

### Example 2: Phase 3 Interventional Study using Atorvastatin to Reduce Cisplatin-Induced Hearing Loss among Individuals with Head and Neck Cancer

### Rationale for Study Design

In the retrospective/observational prospective cohort, 54.7% of all adults treated with cisplatin for head and neck cancer developed a clinically meaningful hearing loss, per CTCAEv5.0 ototoxicity criteria. In the proposed interventional study, enrolled participants given the placebo will be similarly vulnerable to a cisplatin-induced hearing loss. Hearing losses range in severity from mild (<15 dB threshold shift) to severe (>70 dB threshold shift) and are irreversible. The observational data show that those on once daily atorvastatin were 45% less likely to develop a cisplatin-related hearing loss relative to those not on atorvastatin (OR 0.55, 95% CI: 0.327-0.942). This study is designed to determine the extent to which 20 mg atorvastatin reduces cisplatin-induced hearing loss. In total, the study duration will be 48 months of active study enrollment+2 years to obtain follow-up survival data and participant duration will be 7 months with up to 2 years follow up.

In order to be eligible to participate in either the observational or interventional arms of this study, an individual must meet all of the following criteria as evaluated by the study team, including an on-site oncologist: 1) willingness and ability to comply with and participate in all study procedures and availability for the duration of the study; 2) must be an adult patient, male or female, aged ≥18; 3) diagnosed with squamous cell carcinoma of the head and neck, confirmed by pathologic review of surgical or biopsy specimen(s), who meets standard clinical and laboratory criteria and underwent treatment with concomitant cisplatin-based chemotherapy and radiation with curative intent (this includes patients who were treated with either intensity-modulated radiation therapy (IMRT) or proton radiotherapy, with planned dose to the cochlea of <35 Gy (to limit confounding effects of radiation; Bhandare et al., 2010)); 4) subjects must have hearing thresholds at or better than 70 dB SPL at 1, 2, and 4 kilohertz (kHz) at the time of their baseline audiogram; and 5) the ability to provide consent and provision of signed and dated informed consent form.

Further, in order to be eligible to participate in the interventional arm of this study, an individual must meet all of the following criteria: 1) baseline laboratory tests in the following range: aspartate aminotransferase (AST or SGOT); alanine aminotransferase; creatine phosphokinase, creatinine <1.5× ULN; 2) the ability to take oral medication by mouth or by feeding tube willingness to adhere to the daily atorvastatin or placebo regimen; and 3) for females of reproductive potential, use of highly effective contraception for at least 1 month prior to enrollment and agreement to use such a method during study participation and for an additional 8 weeks after the end of atorvastatin administration. The investigators will attempt to enroll subjects of diverse race, gender and age. However, the ability to do this will be limited by the demographics of patients typically diagnosed with HNSCC. There is a male predominance, and patients tend to be in their 5th to 7th decades of life (“Cancer of the Oral Cavity and Pharynx-Cancer Stat Facts,” n.d.).

Individuals who meet any of the following criteria will be excluded from participation in the observational or interventional arms of this study: 1) patients with a Type B tympanogram; 2) patients with bilateral cochlear implants; 3) patients with a history of prior treatment with platinum chemotherapy drugs; 4) patients for whom additional adjuvant platinum-based chemotherapy is planned after the completion of concomitant chemoradiation (e.g., patients with nasopharyngeal carcinoma); 5) staff members of the NIDCD Sections and of the lead site investigators that are headed by the PI and LAI; and 6) children because HNSCC in children under age 18 is exceedingly rare. Further, individuals who meet any of the following criteria will be excluded from participation in the interventional arm of this study: 1) current use of cimetidine, spironolactone, ketoconazole, cyclosporine, or protease inhibitors for HIV or hepatitis C; 2) pregnancy, lactation, or plan to become pregnant; 3) known allergic reactions to components of atorvastatin or the placebo; and 4) other severe or unstable medical conditions for which clinical site PI believed will increase risk to safety or being able to complete study.

Patients who are unable to provide consent will be excluded because the aims of the study can be achieved by only including those who can consent for themselves. Pregnant women will not be excluded from the observational arm of the study if they meet all of the other inclusion criteria, even though they are unlikely to be treated with cisplatin. The criteria for including pregnant women and fetuses in research, 45CFR46, Subpart B, #-46.204 have been reviewed. The risk for pregnant women (and their fetuses) to participate in the observational arm of this study is minimal, with the risk to the fetus less than minimal as they will not participate in the audiogram or tympanogram, and the hearing test could provide direct benefit to the pregnant woman, plus meet all the other guidelines as noted. However, pregnant women will be excluded from the interventional arm of the study. Atorvastatin is classified as a Category X drug by the Food and Drug Administration, which signifies that studies have shown they may cause birth defects and that the risks clearly outweigh any benefit.

Furthermore, individuals who do not meet the criteria for participation in either arm of this trial (screen failure) will be excluded. Additionally, individuals who do not meet criteria for randomization to atorvastatin or placebo will be invited to enroll in the observational arm of the study.

### Known Atorvastatin Potential Risks and Benefits

Risk statements taken from the atorvastatin package insert include: 1) the five most common adverse reactions in patients treated with atorvastatin that led to treatment discontinuation and occurred at a rate greater than placebo were: myalgia (0.7%), diarrhea (0.5%), nausea (0.4%), alanine aminotransferase increase (0.4%), and hepatic enzyme increase (0.4%); 2) rare cases of rhabdomyolysis with acute renal failure secondary to myoglobinuria have been reported with atorvastatin calcium and with other drugs in this class. A history of renal impairment may be a risk factor for the development of rhabdomyolysis; 3) atorvastatin, like other statins, occasionally causes myopathy, defined as muscle aches or muscle weakness in conjunction with increases in creatine phosphokinase (CPK) values >10 times the upper limit of reference range. The concomitant use of higher doses of atorvastatin with certain drugs such as cyclosporine and strong CYP3A4 inhibitors (e.g., clarithromycin, itraconazole, and HIV protease inhibitors) increases the risk of myopathy/rhabdomyolysis; 4) statins, like some other lipid-lowering therapies, have been associated with biochemical abnormalities of liver function. Persistent elevations (>3 times the upper limit of normal [ULN] occurring on 2 or more occasions) in serum transaminases occurred in 0.7% of patients who received atorvastatin calcium in clinical trials. The incidence of these abnormalities was 0.2%, 0.2%, 0.6%, and 2.3% for 10, 20, 40, and 80 mg, respectively; 5) liver function tests will be performed prior to and at 12±3 weeks following the initiation of therapy, and periodically (e.g., semiannually) thereafter. Liver enzyme changes generally occur in the first 3 months of treatment with atorvastatin calcium. Patients who develop increased transaminase levels should be monitored until the abnormalities resolve. Should a clinically significant increase in ALT or AST persist, withdrawal of atorvastatin calcium is recommended; 6) atorvastatin calcium will be used with caution in patients who consume substantial quantities of alcohol and/or have active liver disease or a history of liver disease, which are potentially increased in HNSCC associated with increased tobacco and alcohol use; 7) statins interfere with cholesterol synthesis and theoretically might blunt adrenal and/or gonadal steroid production. Clinical studies have shown that atorvastatin calcium does not reduce basal plasma cortisol concentration or impair adrenal reserve. The effects of statins on male fertility have not been studied in adequate numbers of patients. The effects, if any, on the pituitary-gonadal axis in premenopausal women are unknown. Caution will be exercised if a statin is administered concomitantly with drugs that may decrease the levels or activity of endogenous steroid hormones, such as ketoconazole, spironolactone, and cimetidine; and 8) atorvastatin calcium is contraindicated in women who are or may become pregnant. It is not known whether atorvastatin is excreted in human milk, but a small amount of another drug in this class does pass into breast milk.

Known benefits of atorvastatin include (from prescribing information): 1) reduced risk of myocardial infarction (MI), stroke, revascularization procedures, and angina in patients without coronary heart disease (CHD), but with multiple risk factors; 2) reduced risk of MI and stroke in patients with type 2 diabetes; 3) reduced risk of non-fatal MI, fatal and non-fatal stroke, revascularization procedures, hospitalization for congestive heart failure (CHF) and angina in patients with CHD; and 4) reduced elevated total-C, LDL-C, apo B, and TG levels and increase in HDL-C levels in patients with primary hyperlipidemia. Potential benefits of atorvastatin may include reduced incidence and/or severity of cisplatin-induced hearing loss.

### Assessment of Potential Risks and Benefits: Rationale for the Necessity of Exposing Participants to Risks

The retrospective study in humans demonstrates that 57.3% of patients treated for HNSCC with cisplatin-based chemoradiation therapy (CRT) will experience a permanent hearing loss of sufficient severity (CTCAEv5.0 grade 1 or higher) to adversely impact communication ability. Thus, there is a major unmet clinical need for a therapy to reduce this cisplatin-induced hearing loss. The proposed therapy being tested is atorvastatin, an FDA-approved drug with a long history, good tolerability, and an excellent safety profile in humans.

Risks to participants were minimized in the study design. First, potential subjects who are at increased risk for adverse effects of atorvastatin (pregnant women, those who may become pregnant, nursing mothers, those with liver or kidney disease, those who consume substantial amounts of alcohol, those taking ketoconazole, spironolactone, cimetidine, cyclosporine, clarithromycin, itraconazole, or protease inhibitors for HIV or hepatitis C) will not be enrolled into the interventional arm of the study (and thus will not be administered atorvastatin). However, these subjects will be invited to enroll in the observational arm of the study if they are otherwise eligible. Thus, all subjects who are eligible to enroll in the interventional arm of the study are considered low risk for significant adverse effects of atorvastatin. Secondly, the selected dose of atorvastatin is one of the lowest doses available, and will be limited to duration of administration during cisplatin therapy, thus reducing the risk of some adverse events (e.g., myopathy, rhabdomyolisis) that were specifically observed with higher doses and prolonged use. Furthermore, prior to enrollment in the interventional arm of the study, subjects will undergo liver function testing. This testing will be repeated approximately 12 weeks+3 weeks after initiation of atorvastatin (or placebo) therapy. Enrolled subjects with persistent, clinically significant elevations in hepatic transaminase or CPK or creatinine levels will be taken off atorvastatin and will be referred for clinical follow up with an appropriate care provider.

Data from an observational study in 277 (statin use (N=113); non-statin use(N=164)) patients undergoing cisplatin-based chemoradiation therapy (CRT) to treat head and neck squamous cell carcinoma (HNSCC) patients indicate that those taking atorvastatin had significantly reduced incidence and severity of hearing loss compared to patients not taking a statin drug (NIH study #17-DC-0138).

### Justification for Dosage

Data collected from 50 atorvastatin users (97 ears) at doses ranging from 10-80 mg tablets indicate that all doses of atorvastatin are equally effective at reducing cisplatin-induced hearing loss in individuals with head and neck cancer (FIG. 8). Though the risks of atorvastatin are low, a low dose (20 mg) was selected to reduce the incidence of potential side effects. The Phase 3 study utilizes 20 mg atorvastatin based on the statistically significant and clinically powered (p>0.8) data from the retrospective/observational prospective study. Once-daily atorvastatin at 20 mg is considered safe and is well tolerated clinically amongst adults (Beckwitt et al., 2018; Gupta et al., 2019; Jian-Yu et al., 2018; Lebo et al., 2018; Lee et al., 2016; Seckl et al., 2017; Ung et al., 2018b; Xie et al., 2017).

Specifically, per CTCAEv5.0 criteria, 57.3% of patients who were not taking statins developed a clinically significant cisplatin-induced hearing loss in at least one ear that would be expected to impact communication ability. In contrast, only 50.1% of those on a statin drug during therapy were identified as acquiring a CTCAEv5.0-grade hearing loss following treatment with cisplatin. Of the 113 statin users, atorvastatin and simvastatin were the two most-represented statins in our cohort, at 45% and 32%, respectively. These drugs were also analyzed in isolation. The incidence of a CTCAEv5.0-grade hearing loss was further reduced with atorvastatin use to 46%. A mixed effect model analysis identified cumulative cisplatin dose (p<0.001), pre-existing hearing loss (p<0.001) and atorvastatin use (p<0.02) as significant predictors of hearing loss due to cisplatin exposure. After controlling cumulative cisplatin dose and pre-existing hearing loss, atorvastatin users had significantly reduced incidence of cisplatin-induced hearing loss compared to patients not taking a statin (CTCAEv5.0: OR=0.49, 95% CI: 0.30-0.82, p<0.01). In contrast, simvastatin was not protective with 63.9% of users developing significant hearing loss. Therefore, a Phase 3, multi-institutional, randomized, placebo-controlled, double blinded, interventional trial was conducted to determine the extent to which atorvastatin reduces cisplatin-induced hearing loss in adults treated with cisplatin-based CRT for head and neck cancer.

The primary objective (Interventional Arm) is to determine the effectiveness of atorvastatin (20 mg) at reducing the incidence of a CTCAEv5.0 Grade ≥2 hearing loss in patients treated with cisplatin-based CRT for head and neck squamous cell carcinoma (HNSCC). Secondary objective (Observational Arm) is to examine the extent to which subjects taking other statin drugs, and other doses of atorvastatin, exhibit reduced incidence CTCAEv5.0 grade ≥2 hearing loss in patients treated with cisplatin-based CRT for head and neck squamous cell carcinoma (HNSCC). The tertiary objectives (Interventional Arm) are to determine if: 1) concomitant atorvastatin (20 mg) use alters disease-free survival or 2) overall survival in subjects undergoing cisplatin-based CRT.

The primary endpoint (Interventional Arm) is the change in hearing sensitivity as measured by pure-tone audiometry between the pre-treatment (before cisplatin-based CRT) hearing test and the post-treatment (after completion of cisplatin-based CRT) audiogram. Hearing loss will be defined according to CTCAEv5.0 Grade ≥2 criteria and will be compared in subjects taking atorvastatin vs. subjects not taking any statin drug. Hearing sensitivity will be compared between audiograms collected at baseline prior to treatment to a repeated audiogram at the end of treatment within 2-4 months of cessation of cisplatin administration. The justification for this endpoint is that audiometric testing is the gold standard for measuring changes in hearing sensitivity. The CTCAEv5.0 grading scale was selected because it is designed to identify a clinically-meaningful change in hearing. Data from observational and retrospective studies indicate that cisplatin-induced hearing loss is detectable at 2-4 months after cessation of cisplatin therapy.

Secondary endpoint (Observational Arm) is the change in hearing sensitivity as measured by pure-tone audiometry between the pre-treatment (before cisplatin-based CRT) hearing test and the post-treatment (within 2-4 months of cessation of cisplatin administration) audiogram in the observational arm of the study. Hearing is defined according to CTCAEv5.0 Grade ≥2 criteria and will be compared in subjects taking: 1) any statin other than atorvastatin vs. subjects not taking any statin drug, and 2) subjects taking atorvastatin at doses other than 20 mg vs. subjects not taking any statin. Hearing sensitivity will be compared between audiograms collected at baseline prior to treatment to a repeated audiogram at the end of treatment within 2-4 months of cessation of cisplatin administration. The justification for this endpoint is that audiometric testing is the gold standard for measuring changes in hearing sensitivity. The CTCAEv5.0 grading scale was selected because it is designed to identify a clinically-meaningful change in hearing. Data from observational and retrospective studies indicate that cisplatin-induced hearing loss is detectable at 2-4 months after cessation of cisplatin therapy.

The tertiary endpoint (Interventional Arm) is the overall and disease-free survival at 2 years after cisplatin-based CRT. Overall survival and disease-free median survival will be compared between subjects taking atorvastatin (20 mg) vs. those not taking any statin. The justification for this endpoint is because it is important to confirm that atorvastatin use (and other statins in the observational arm) does not reduce overall response, survival or disease-free survival in patients with HNSCC. To date, only two retrospective studies have compared survival in HNSCC patients taking statins (Saadah, 1993).
414 subjects with newly-diagnosed HNSCC scheduled to undergo cisplatin-based chemotherapy with concurrent radiation will be recruited from three to four U.S. based cancer centers providing standard-of-care therapy for in head and neck cancer. These centers include, National Institutes of Health (NIH) Clinical Center, Wilmot Cancer Institute at the University of Rochester Medical Center in New York, the Winship Cancer Institute at Emory University in Georgia, and possibly the University of Maryland Medical Center in Maryland.

Based on the retrospective data, approximately 40% of these subjects will already be on a statin medication and, therefore, these subjects will be assigned to the observational arm of the study. The remaining 60% will be assessed for eligibility for the interventional arm. Subjects enrolled in the interventional arm will be randomized to receive either once daily atorvastatin (20 mg), or placebo. Subjects enrolled in the interventional arm and the investigators conducting the data analysis will be blinded to the drug designation of each subject. Prior to onset of cisplatin treatment and within 2-4 months following cessation of cisplatin treatment, enrolled subjects at all sites will undergo a hearing evaluation consisting of pure tone audiometry (air and bone conduction thresholds) and tympanometry (see Table 8). When feasible, data collection sessions will be scheduled during outpatient consultation, radiation, or follow up visits at each participating clinical site. It is hypothesized that there is a difference in the distribution of CTCAEv5.0-defined hearing loss between atorvastatin (20 mg) and placebo groups, i.e., the distribution of responses depends on the group.

TABLE 8
Schedule of Activities (SOA)

| | | | | | | | | | | | | | Follow |
|---|---|---|---|---|---|---|---|---|---|---|---|---|---|
| | | | | | | | | | | | | | up/Study |
| | Screening | | Standard of Care Cisplatin-based | | | | | | | | | Follow up | Endpoint 2 |
| | Day −90 | Enrollment/ | chemoradiation therapy (CRT) | | | | | | | | Wk 12 ± | 2-4-month | yrs ± 6 mo |
| Procedures | to 1 | Baseline | Wk 1 | Wk 2 | Wk 3 | Wk 4 | Wk 5 | Wk 6 | Wk 7 | Wk 8 | 3 wks. | post CRT | post CRT |
| Informed consent | X | | | | | | | | | | | | |
| Chart Review | | X | | | | | | | | | | | X |
| Study arm designation, | | X | | | | | | | | | | | |
| observational vs. | | | | | | | | | | | | | |
| interventional | | | | | | | | | | | | | |
| Serum chemistry^{a} | | X | | | | | | | | | X | | |
| (interventional arm only) | | | | | | | | | | | | | |
| Pregnancy test^{b} | | X | | | | | | | | | | | |
| (interventional arm only) | | | | | | | | | | | | | |
| Randomization | | X | | | | | | | | | | | |
| (interventional arm only) | | | | | | | | | | | | | |
| Auditory evaluation | | X | | | | | | | | | | X | |
| (hearing test and | | | | | | | | | | | | | |
| tympanometry) | | | | | | | | | | | | | |
| Administer study | | X | X | X | X | X | X | X | X | X | | | |
| intervention | | | | | | | | | | | | | |
| (interventional arm only) | | | | | | | | | | | | | |

^{a}Albumin, alkaline phosphatase, total bilirubin, bicarbonate, BUN, calcium, chloride, creatine phosphokinase (CPK), creatinine, glucose, LDH, phosphorus, potassium, total protein, AST, ALT, sodium.
^{b}Serum pregnancy test (women of childbearing potential).

Eligible subjects will be tentatively assigned to a study arm based on existing statin medication use and eligibility criteria. Those on any statin drug or contraindications for atorvastatin use will be assigned to the observational arm of the study. Their hearing will be reassessed 2-4 months after cessation of cisplatin treatment, and medication use will be verified. Their data will be analyzed to determine the effectiveness of statin medications at reducing the incidence and severity of cisplatin-induced hearing loss. Individuals that are not on a statin medication at the time of enrollment will be screened (including blood tests for liver and kidney functions) for their eligibility for the interventional arm of the study. Subjects enrolled in the interventional arm of the study will be randomized to receive either atorvastatin (20 mg) or a placebo. Their hearing will then be reassessed 2-4 months after cessation of cisplatin treatment, medication use verified, and their data will be analyzed to determine the effectiveness of atorvastatin 20 mg at reducing the incidence and severity of cisplatin-related hearing loss relative to placebo users.

Subjects enrolled in the study will be active participants for approximately 30 months from the time of their baseline hearing evaluation, through their cisplatin treatment, and to their final follow up evaluation within 2-4 months of cessation of cisplatin administration. Their medical records will be reviewed at 2 yrs+/−6 mo. post cessation of treatment to obtain survival data. Total time of enrollment may vary based on the recommended standard-of-care follow-up schedule of each subject. Study subjects will undergo two hearing tests-one prior to cisplatin administration, and one after cessation of cisplatin administration.

### Study Intervention

For the interventional arm of the study, subjects will be provided with atorvastatin (20 mg) or placebo to be taken daily by mouth or by feeding tube. The tablets may be taken whole or crushed according to patient swallowing capabilities and preference. Atorvastatin will be administered at a low dose of 20 mg, which has a very low incidence of adverse effects (Bakker-Arkema et al., 1996).

### Dose Escalation

Atorvastatin (20 mg) or placebo will be taken by mouth or by feeding tube daily. The drug or placebo can be taken at any time of day, with no specified relation of dosing to meals. Atorvastatin or placebo will be initiated upon enrollment, continued during chemoradiation treatment (6-7 weeks), then continued for an additional 2-4 months following the completion of chemoradiation, for a total of up to seven months. There will be no escalation or reduction of the atorvastatin dose. There will be no dose modifications. However, dose limiting toxicities will be addressed by stopping atorvastatin or placebo.

### Acquisition and Accountability

Atorvastatin 20 mg and placebo will be formulated by Pine Pharmaceuticals, an FDA drug compounding pharmacy (FDA-registered 503B Outsourcing Facility). The active and placebo drugs will be formulated under strict cGMP and supplied in an appropriately sized gelatin capsule, utilizing appropriate excipients to ensure identical appearance between placebo and active. The active capsules will be formulated utilizing a trituration manufactured with the commercially available drug product. Composition, storage, and stability of both the active and placebo will be tested by an independent analytical laboratory and evaluated out to one year past the date of manufacturing. Pine will ship the active (atorvastatin) and control (placebo) products directly to the collaborating (distributing) pharmacy at each study site.

### Formulation, Appearance, Packaging, and Labeling

Commercially-available atorvastatin is a white tablet, and the placebo will be formulated to also contain a white powder such that the atorvastatin and placebo are indistinguishable even if a capsule was opened. The capsules will be packaged in suitable pharmaceutical packaging. Labeling was in accordance with the Guidance for Industry for 503B Outsourcing Facilities. Additionally, the containers holding drug product will be blinded.

### Product Storage and Stability

Both the active and placebo were stored at room temperature, and stability was assessed concurrently with the study. Stability of both the active and placebo were evaluated out to one year past the date of manufacturing.

### Preparation

Both atorvastatin (20 mg) and placebo will be suppled as capsules that will not require additional preparation by study staff and/or study participants so long as the study participants are able to swallow the capsules. Participants who develop swallowing difficulty due to their disease may need to open the study capsules and mix the contents of the capsule into a food that was easy to swallow (i.e., applesauce, pudding). The capsules will be prepared such that the contents of the atorvastatin and placebo capsules have identical appearances, the opening the capsule will not result in unblinding of the participant.

### Measures to Minimize Bias: Randomization and Blinding

Subjects currently taking a statin drug will be assigned to the observational arm of the study. Subjects not currently taking a statin will be assigned to the interventional arm of the study and then randomized to atorvastatin or placebo. Based on prior retrospective studies, it is estimated that ˜60% of eligible subjects will not be taking statin drugs at enrollment and, therefore, will be screened for eligibility for the interventional study.

Atorvastatin and placebo have an identical appearance, and subjects will remain blinded. The PI and lead AI at NIH will be involved in data analysis and also remain blinded to the study intervention. The participating pharmacy as well as investigators with lipid expertise, otolaryngology and oncology will be unblinded due to the need for drug safety monitoring. These unblinded investigators will not share laboratory values or randomization information with patients, the PI or the lead AI unless there is a concern for drug toxicity. If the unblinded investigators who are monitoring safety determine that a dose-limiting toxicity or AE has occurred, the PI will be unblinded to the randomization of that particular subject in order to facilitate reporting. Any intentional or unintentional unblinding will be reported to the PI, and another investigator will be recruited to oversee the data analyses.

Trial randomization codes will be maintained by a designated investigator or clinical research staff member at each study site. Unless an AE or DLT occurs, randomization codes will only be broken after 188 subjects had been randomized in the interventional arm of the study and completed their first post-cisplatin audiogram.

### Study Intervention Compliance

Clinical research staff will attempt to contact each subject weekly by phone or in person at scheduled clinic visits to review the drug log and assess compliance. Any missed doses reported by subjects will be recorded to estimate the percent compliance.

### Concomitant Therapy

For this protocol, a prescription medication is defined as a medication that can be prescribed only by a properly authorized/licensed clinician. Medications to be reported in the Case Report Form (CRF) are concomitant prescription medications, over-the-counter medications and supplements. These will be reviewed and recorded at each study visit. Although higher doses of atorvastatin may be more likely to cause rhabdomyolysis/myopathy when given concomitantly with CYP3A4 inhibitors, this complication is rare with the 20 mg dose of atorvastatin (Bakker-Arkema et al., 1996).

### Discontinuation of Study Intervention

In the interventional arm, discontinuation from atorvastatin 20 mg or placebo will equate to discontinuation of study participation. Participants who are unable or unwilling to take the study intervention (atorvastatin or placebo) ≥80% of the time will be removed from the study and replaced.

If a clinically significant finding is identified (including, but not limited to changes in liver function or myopathy) after enrollment, study investigators will determine if any change in participant management is needed. Due to the low and distinct risk profile related to hepatic function or myopathy attributable to study agent minimal adverse events are expected. Grade 3 skin, mucosal and Grade 3/4 hematologic toxicities of cisplatin and radiation are common, but Grade 4 skin, mucosal and Grade 4 hematologic, except for lymphopenia, observed in the interventional arm would merit stoppage of the intervention to discern possible relationship to the combination. Any new clinically relevant finding and attribution will be reported as an adverse event.

The data to be collected at the time of study intervention discontinuation will include the following: 1) the dates the participant started and ended the study intervention (atorvastatin or placebo); and 2) the rationale for discontinuation of the study intervention.

Participants are free to withdraw from participation in the study at any time upon request. An investigator may discontinue or withdraw a participant from the study for the following reasons: 1) completion of study intervention; 2) disease progression which requires discontinuation of the study intervention; 3) if any clinical adverse event (AE), laboratory abnormality, or other medical condition or situation occurs such that continued participation in the study would not be in the best interest of the participant; 4) investigator discretion; 5) positive pregnancy test; and 6) participant who are unable or unwilling to take the study intervention (atorvastatin or placebo) ≥80% of the time.

### Screening Procedures

All study procedures will be conducted at all approved study sites. Patients newly diagnosed with head and neck squamous cell carcinoma (HNSCC) will be screened to meet study eligibility criteria by a study physician or coordinator at each site. All study participants will need to be 18 years or older and scheduled to receive cisplatin-based chemotherapy with concurrent radiotherapy. Once an informed consent is obtained, subjects will be screened for eligibility for the observational vs. interventional arms of the study (i.e., current statin use). If subjects are deemed potentially eligible for the interventional arm of the study, a blood sample will be collected prior to start of cisplatin-treatment to assess each participant's preexisting risk for liver, muscle, and kidney damage. Throughout the course of the study, the patient's chart will be reviewed for data collection relevant to concomitant medications and cancer treatment details in accordance with the Health Insurance Portability and Accountability Act (HIPAA), as well as federal, state and local institutional requirements.

### Efficacy Assessments

Blood Sample Collection: For subjects deemed potentially eligible for the interventional arm of the study, blood samples will be collected after informed consent has been obtained, prior to the start of cisplatin treatment to assess each participant's preexisting risk for liver, muscle, and kidney damage.

Otoscopy: An otoscopic exam will be done in the event a tympanogram is abnormal (flat, Type B with peak compliance <0.3 ml) or unobtainable by a licensed Audiologist, Otologist, or other previously identified study personnel.

Tympanogram: Prior to each audiogram, a tympanogram will be performed. These tests can be administered to subjects by on-site study coordinators and/or study nurses and do not require administration by someone with a background in advanced auditory assessment. In the event a tympanogram cannot be obtained or if the results of the tympanometry screen are abnormal (flat, Type B with peak compliance <0.3 ml) (Davies, 2016; Onusko, 2004; The Otitis Media Guideline Panel, 1994), a licensed Audiologist, Otologist, or other previously identified study personnel will conduct otoscopy and determine the proper course of action. If a tympanogram is abnormal, the subject will be examined by an otolaryngologist or audiologist to rule out cerumen impaction. If cerumen impaction is noted, the cerumen will be removed, and the tympanogram will be repeated. Subjects with an unresolved Type B tympanogram will remain in the study; however, the results of their tympanogram will be noted in their record and taken into consideration during data analysis.

Audiogram: An audiogram will be performed by a study coordinator at each site during the baseline evaluation and again at the follow up evaluation within 2-4 months of completing cisplatin treatment. A final audiogram will be obtained 2 years (+6 months) after cessation of cisplatin therapy. Pure tone air conduction thresholds will be collected individually for the right and left ears for standard audiometric frequencies including 1, 2, 3, 4, 6, and 8 kHz as well as for extended high frequencies 10 and 12.5 KHz.

Drug administration: Subjects enrolled in the interventional arm will be prescribed either a placebo or atorvastatin (20 mg) to be taken once daily by mouth or through a feeding tube throughout their 6-8 week cisplatin treatment as well as through to the 2-4 month post-CRT follow up hearing test.

### Safety and Other Assessments

In the interventional arm of the study, blood samples will be collected after informed consent has been obtained, prior to the start of cisplatin treatment to assess each participant's preexisting risk for liver, muscle, and kidney dysfunction. All blood samples will be collected and processed with commercially available assays at each respective site and reviewed by an investigator on the protocol. Laboratory values outside the reference range will be reviewed by a physician with appropriate lipid expertise to determine eligibility. Laboratories will assess muscle with creatine phosphokinase (CPK) or creatine kinase (CK), liver with a hepatic panel (AST, ALT, alkaline phosphatase, bilirubin), renal function with blood urea nitrogen (BUN) and creatinine (Cr). All blood tests will be Clinical Laboratory Improvement Amendments (CLIA) approved. In most cases, all tests can be done on one tube of blood (4 ml). Subjects found to have clinically significant results at baseline (e.g., evidence of pre-existing liver disease) will be excluded from the study and referred to a clinical provider with appropriate expertise to institute further diagnostic workup and treatment, which will not be provided under this protocol.

Counseling procedures, including any dietary or activity considerations that need to be adhered to during study participation: each subject will be asked to keep a drug log to track daily doses of atorvastatin/placebo. Clinical research staff will attempt to contact each subject weekly by phone or in person at scheduled clinic visits to review the drug log and assess compliance. Any missed doses reported by subjects will be recorded to estimate the percent compliance.

Assessment of adverse events: subjects will not be able to obtain their hearing test results at the completion of each evaluation. Subjects will be notified of the results of their hearing testing after they have completed chemoradiation therapy and have had their follow up evaluation (or when they choose to withdraw).

### Adverse Events and Serious Adverse Events

The National Cancer Institute (NCI) published CTCAEv5.0 to be used as a grading (severity scale) to apply descriptive terminology to Adverse Event (AE) reporting. Under their classification system, hearing impairment falls under the category of an AE and can be categorized on a 0 to 4 Grade Scale. Table 9 shows the criteria applicable for adults enrolled in an ototoxicity monitoring program where air conduction thresholds are obtained on a 1, 2, 3, 4, 6, and 8 kHz audiogram.

TABLE 9
Common Terminology Criteria for Adverse Events version
5.0 (CTCAEv5.0) Criteria for Ototoxic Hearing Loss

| Grade | | |
|---|---|---|
| Assignment | Severity Description | Grade Criteria |
| Grade 0 | No hearing loss | — |
| Grade 1 | Mild; asymptomatic or | Threshold shift of 15-25 dB |
| | mild symptoms; clinical | averaged at 2 contiguous test |
| | or diagnostic | frequencies in at least one ear |
| | observations only; | |
| | intervention not | |
| | indicated | |
| Grade 2 | Moderate; minimal, | Threshold shift of >25 dB |
| | local or noninvasive | averaged at 2 contiguous test |
| | intervention indicated | frequencies in at least one ear |
| Grade 3 | Severe or medically | Threshold shift of >25 dB |
| | significant but not | averaged at 3 contiguous test |
| | immediately | frequencies in at least one ear |
| | life-threatening | |
| Grade 4 | Life-threatening | Decrease in hearing to |
| | consequences; urgent | profound bilateral loss |
| | intervention indicated | (absolute >80 dB HL at 2 kHz |
| | | and above) |

Common Terminology Criteria for Adverse Events (CTCAEV5.0) | Protocol Development | CTEP [WWW Document], n.d. URL
https://ctep.cancer.gov/protocolDevelopment/electronic_applications/ctc.htm#ctc_50 (accessed Dec. 29 2019).

AEs and severe AEs are common for subjects on cisplatin and RT, and unblinding for all of these would not be appropriate and would compromise the study. Elevated liver enzymes, CK, myalgias, other known side effects of grade ¾ or greater possibly related to atorvastatin or combination can be addressed by stopping statin or placebo first. Those serious that persist >1 week after statin stoppage and potentially also attributable to cisplatin or radiation will be addressed by dose modification or treatment as clinically indicated for those therapies. This will be determined by the treating oncologist(s). Adverse events that are expected as a result of HNSCC, cisplatin and/or radiation (e.g., nausea, vomiting, mucositis, peripheral neuropathy, dysphagia, xerostomia, fatigue, pain at the tumor site) will not be reported.

### Statistical Considerations

Primary Endpoint(s): To determine the effectiveness of atorvastatin (20 mg) at reducing the incidence of cisplatin-induced hearing loss in patients with head and neck squamous cell carcinoma (HNSCC).

H₀: The distribution of the outcome defined by CTCAEv5.0 Grade ≥2 hearing loss is independent of the groups of atorvastatin users relative to subjects on a placebo.

H₁: There is a difference in the distribution of CTCAEv5.0 Grade>2 hearing loss between atorvastatin and placebo groups, i.e., the distribution of responses depends on the group.

### Sample Size Determination

Auditory threshold shifts will be calculated based on the difference in audiological thresholds between pre- and post-cisplatin treatment audiograms over a 1 to 12.5 KHz frequency range. Common Terminology Criteria for Adverse Events (CTCAEv5.0) ototoxicity scale criteria will be applied to threshold shifts ranging from 1 to 8 kHz to identify clinically meaningful changes in hearing. Additionally, no data points will be calculated/replaced in the event of missing test points.

The proportion of a CTCAEv5.0 Grade ≥2 hearing loss among atorvastatin (20 mg) and placebo after CRT will be the primary outcome measure. A Grade 2 hearing change is defined as >25 dB average change across two consecutive frequencies from 1 to 8 Hz. A CTCAEv5.0 Grade ≥2 change is considered a moderate adverse event (National Cancer Institute (NCI), 2017). The incidence of a CTCAEv5.0 Grade ≥2 hearing loss relative to atorvastatin (20 mg) use will be assessed using a Z test for the equality of two proportions. The chi-square test will be used to examine differences in the severity grade distribution of CTCAEv5.0-defined hearing loss among atorvastatin (20 mg) and placebo users (categorical variables).

A multivariable logistic regression analysis with calculation of odds ratios and 95% confidence interval will be used to assess associations between hearing loss and atorvastatin (20 mg) treatment after adjustment for selected covariates. Covariates in the model will include cumulative cisplatin dose, pre-existing hearing loss, age, sex, race/ethnicity, and radiation exposure.

Two-sided tests and a 5% significance level will be employed.

Criteria for significance: Alpha=0.05 with 90% power.

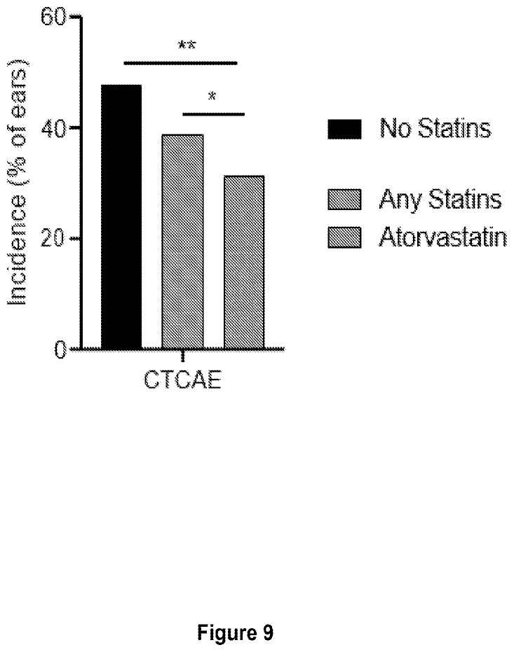

Preliminary data from retrospective/observational prospective study (n=277 subjects with head and neck cancer obtained in collaboration with University of Rochester Medical Center, Walter Reed National Military Medical Center, and Johns Hopkins University) have been used as the basis for statistical predictions and sample size estimates in the current protocol (see Table 10 and FIG. 9). Audiological threshold information collected before and after CRT was analyzed in the same manner as described in this protocol to determine if the concurrent use of statin medications altered the incidence of a hearing loss due to ototoxic medication.

TABLE 10
Preliminary Data From Retrospective/Observational Prospective Study Used as
Basis for Statistical Predictions and Sample Size Estimates in Phase 3 Study

| | No Statin | | Any Statin | | Atorvastatin | |
|---|---|---|---|---|---|---|
| | (n = 164) | | (n = 113) | | (n = 50) | |
| Median Age (IQR), yr | 58 | (50-63) | 63 | (58-68) | 63.5 | (58-67) |
| Male, No (%) | 136 | (82.93) | 96 | (85.71) | 42 | (85.71) |
| Median cisplatin dose | 200 | (155-280) | 200 | (135-280) | 240 | (160-280) |
| (IQR), mg/m² | | | | | | |
| Radiation, No. (%) | 161 | (98.2) | 111 | (99.11) | 49 | (100) |

Based on a chi-square analysis and a linear regression analysis of the aforementioned data controlling for cumulative cisplatin dose, sex, age, radiation, and pre-existing hearing loss, a statistical significance was determined using CTCAEv5.0 and TUNE criteria to compare changes in hearing in atorvastatin users and non-statin users (p<0.03, power >0.8).

A power analysis was performed to determine the sample size needed for tests of two independent proportions using G*Power Version 3.1.9.6 (Faul et al., 2009, 2007). Main comparison will be between subjects taking atorvastatin (20 mg) vs. those taking placebo. Based on the retrospective data, ˜37.7% hearing loss in the placebo group and ˜16.7% hearing loss in the atorvastatin (20 mg) group (CTCAEv5.0 Grade ≥2 criteria) was observed. In a 1:1 randomized interventional study, a total sample size of 140 and 186 subjects will allow us to detect a difference in proportion of 0.21 with a power of 0.8 and 0.9 at a two-sided significance of 0.05.

After adjustment for the attrition rate of 25% and the prevalence rate of 40% statin users, the estimated sample sizes are 312 and 414 subjects.

$$\frac{140 / ( 1 - 0.25 )}{1 - 0.4} = 312$$

$$\frac{186 / ( 1 - 0.25 )}{1 - 0.4} = 414$$

Based on an estimated accrual rate of approximately 150 per year target of 414 subjects being reached by the end of year 4.

Furthermore, subjects who do not complete the follow up evaluation will be replaced if there is not yet a statistically significant difference in hearing loss between subjects taking atorvastatin (20 mg) vs. subjects taking the placebo.

### Populations for Analyses

Intention-to-treat (ITT) Analysis Dataset (randomized participants): approximately 60% of subjects meeting eligibility criteria are expected to not already be on a statin medication. These subjects will be directed to the interventional arm of the study and randomized to either receive atorvastatin (20 mg) or placebo. Baseline and follow up data will be obtained, and their concomitant medication use will be verified weekly during their standard of care treatment.

Per-Protocol Analysis Dataset (randomized participants): subjects that have been randomized to the interventional arm of the study will be asked to comply with a daily administration of either atorvastatin (20 mg) or a placebo. Baseline and follow up data will be obtained, and their intervention medication use will be verified weekly during their standard of care treatment. Additionally, a subset analysis will be conducted on subjects who complied sufficiently, defined as at least 80% of study duration, as their data will likely represent the effects of treatment. Their data will be compared against those with 100% compliance to assess partial-use efficacy.

Observational Dataset: approximately 40% of subjects meeting eligibility criteria are expected to already be on a statin medication. These subjects will be directed to the observational arm of the study. Baseline and follow up data will be obtained, and their statin medication use will be verified weekly during their standard of care treatment.

### Evaluable for Toxicity

All patients will be evaluable for toxicity from the time of their first treatment with atorvastatin (20 mg) at 12±3 weeks via a blood sample analysis.

### General Approach to Statistical Analyses

Subject characteristics will be described using median, mean, interquartile range (IQR), and standard deviations for continuous variables and percentages and counts for categorical variables.

Audiological records will be reviewed to include pure tone hearing thresholds for 8 audiometric frequencies ranging from 1 to 12.5 kHz at baseline and follow up evaluations. Threshold shifts will be calculated as the difference in auditory threshold per frequency. No data points will be calculated/replaced in the event of missing test points. In the event there is no response to acknowledge awareness of a pure tone at the highest output level of the audiometer, a value of +5 dB will be added to the highest output level of the audiometer and recorded as the subject's threshold.

CTCAEv5.0 ototoxicity scale criteria will be applied to threshold shifts ranging 1 to 8 kHz to identify clinically meaningful changes in hearing. CTCAEv5.0 incorporates threshold shift data from 1 to 8 KHz and will therefore best describe the functional impact of cisplatin-based CRT on speech related frequencies. Moreover, CTCAEv5.0 is well known by oncologists who ultimately will manage these patients. Other scales, like the TUNE grading system (Theunissen et al., 2014), that incorporate extended high frequency information were considered for this study, but were thought to better serve as ototoxicity monitoring criteria rather than as criteria that could define a change in hearing that would be expected to adversely affect communication ability.

The Z test for the equality of two proportions will be used to examine differences in the incidence of a Grade ≥2 hearing loss and a chi square test will be used to examine the overall severity grade distribution of a CTCAEv5.0-defined hearing losses among atorvastatin (20 mg) and placebo users (categorical variables). Criteria for significance: Alpha=0.05 with 90% power.

A multivariate logistic regression analysis, based on CTCAEv5.0 Grade ≥2 data, with calculation of odds ratios and 95% confidence interval will be used to assess associations between hearing loss and atorvastatin (20 mg) treatment after adjustment for selected covariates. Covariates in the model will include cumulative cisplatin dose, pre-existing hearing loss, age, sex, race/ethnicity and radiation exposure. Criteria for significance: Alpha=0.05.

A mixed effect model analysis on averaged threshold shift data (4-8 kHz) will be used to assess associations between cisplatin-related hearing loss and atorvastatin (20 mg) treatment after adjustment for selected fixed effects while controlling for the use of two ears from each subject as individual observations. Furthermore, fixed effects in the model will include cumulative cisplatin dose, pre-existing hearing loss, age, sex, race/ethnicity and radiation exposure. Criteria for significance: Alpha=0.05, 95% confidence interval.

### Analysis of the Primary Endpoints

The primary endpoint is the difference in incidence of a CTCAEv5.0 Grade ≥2 hearing loss following cisplatin-based CRT comparing subjects taking atorvastatin vs. those taking a placebo for subjects enrolled in the interventional arm of the study. No data points will be calculated/replaced in the event of missing test points. In the event there is no response to acknowledge awareness of a pure tone at the highest output level of the audiometer, a value of +5 dB will be added to the highest output level and recorded as the subject's threshold. Furthermore, hearing loss will be defined as a single endpoint according to CTCAEv5.0 Grade ≥2 criteria and will be compared in subjects taking atorvastatin (20 mg) vs. subjects on a placebo. A Z test for the equality of two proportions will be used to assess differences in the incidence of a Grade ≥2 CTCAEv5.0-defined hearing loss among atorvastatin (20 mg) and placebo users (categorical variables).

Data are entered as frequencies in mutually exclusive categories where one ear may contribute data to one and only one cell in the matrix. Additionally, data will be presented as the overall difference in incidence of a Grade ≥2 CTCAEv5.0-defined hearing loss comparing subjects taking atorvastatin (20 mg) with subjects on a placebo.

### Analysis of the Secondary Endpoint(s)

The secondary endpoint, independent of the primary endpoint, is the difference in incidence of a CTCAEv5.0 Grade ≥2 hearing loss following cisplatin-based CRT for subjects enrolled in the observational arm of the study comparing 1) any statin other than atorvastatin vs. subjects not taking a statin, and 2) subjects taking atorvastatin at doses other than 20 mg vs. subjects not taking a statin.

Data collection for the Secondary Aim (Observational Arm) is designed to capture information on subjects that are using non-atorvastatin statin medications during cancer therapy. While the data on 423 subjects (84 of these subjects are statin users whose statin drug is not atorvastatin) is currently available, additional observational data on these less-common statins will be collected and analyzed in conjunction with existing observational data that was collected over the past 4 years. Because the data collected through the observational arm of this study will complement the ongoing study, a power analysis for these data will not be required and all analyses will be exploratory.

No data points will be calculated/replaced in the event of missing test points. In the event there is no response to acknowledge awareness of a pure tone at the highest output level of the audiometer, a value of +5 dB will be added to the highest output level and recorded as the subject's threshold. Furthermore, hearing loss will be defined as a single endpoint according to CTCAEv5.0 Grade ≥2 criteria and will be compared in subjects taking atorvastatin (20 mg) vs. subjects on a placebo.

The chi-square test will be used to examine differences in the incidence of a Grade ≥2 CTCAEv5.0-defined hearing loss (categorical variables) comparing 1) subjects taking a statin drug other than atorvastatin, and 2) subjects with a longer history of atorvastatin (20 mg) than those in the interventional study arm, 3) subjects taking atorvastatin at doses other than 20 mg vs. subjects on a placebo OR will be combined for a meta-analysis with previously collected retrospective data containing non-atorvastatin (20 mg) statin doses and types. Data are entered as frequencies in mutually exclusive categories where one ear may contribute data to one and only one cell in the matrix.

Criteria for significance: Alpha=0.05 with 90% power.

Data will be presented as the overall difference in the incidence of a Grade ≥2 CTCAEv5.0-defined hearing loss comparing 1) subjects taking a statin medication other than atorvastatin (20 mg), and 2) subjects taking atorvastatin at doses other than 20 mg vs. subjects on a placebo. Furthermore, the overall survival and disease-free survival for subjects on atorvastatin (20 mg) vs. subjects on a placebo will be compared using Mantel-Cox (log rank) tests with alpha=0.05, plotted as survival curves with Kaplan-Meier method over a 2-year monitoring period.

### Safety Analyses

There are no formal safety endpoints in this study. Due to the low and distinct risk profile related to hepatic function or myopathy attributable to study agent minimal adverse events are expected. Grade 3 skin, mucosal and Grade 3/4 hematologic toxicities of cisplatin and radiation are common, but Grade 4 skin, mucosal and Grade 4 hematologic except for lymphopenia observed in the interventional arm would merit stoppage of the intervention to discern possible relationship to the combination. Adverse events will be categorized according to the current CTCAEv5.0 terminology and grading, calculated once for a given participant, and the following information will be reported for each AE: start date, stop date, severity, relationship, expectedness, outcome, and duration. Regulatory reporting will be conducted in accordance with Policy 801.

### Baseline Descriptive Statistics

Subject characteristics will be described using median, mean, interquartile range (IQR), and standard deviations for continuous variables and percentages and counts for categorical variables. Baseline characteristics including age, sex, cumulative cisplatin dose, radiation exposure, and pre-existing hearing loss will be incorporated as covariates in regression and mixed effect models.

### Sub-Group Analyses

A multivariate logistic regression analysis, based on CTCAEv5.0 Grade ≥2 data, with calculation of odds ratios and 95% confidence interval will be used to assess the heterogeneity of treatment effects of atorvastatin (20 mg) across various baseline characteristics. The baseline variables in the model will include cumulative cisplatin dose, pre-existing hearing loss, age, race/ethnicity and sex. The interactions between treatment and baseline variables will be tested. Criteria for significance: Alpha=0.05.

A mixed effect model analysis on averaged threshold shift data (4-8 kHz) will be also used to assess the heterogeneity of treatment effects of atorvastatin (20 mg) among various baseline characteristics while controlling for the use of two ears from each subject as individual observations. Fixed effects in the model will include cumulative cisplatin dose, pre-existing hearing loss, age, race/ethnicity and sex. The interactions between treatment and baseline variables will be tested. Criteria for significance: Alpha=0.05, 95% confidence interval.

A multivariate logistic regression analysis, based on CTCAEv5.0 Grade ≥2 data, with calculation of odds ratios and 95% confidence interval will be used to assess the heterogeneity of treatment effects between 1) statin medications other than atorvastatin (20 mg) and 2) atorvastatin doses other than 20 mg across various baseline characteristics. The baseline variables in the model will include cumulative cisplatin dose, pre-existing hearing loss, age, sex, race/ethnicity and radiation exposure. The interactions between treatment and baseline variables will be tested. Criteria for significance: Alpha=0.05.

Furthermore, a mixed effect model analysis on averaged threshold shift data (4-8 kHz) will be used to assess heterogeneity of treatment effects between 1) statin medications other than atorvastatin (20 mg) and 2) atorvastatin doses other than 20 mg across various baseline characteristics while controlling for the use of two ears from each subject as individual observations. Fixed effects in the model will include cumulative cisplatin dose, pre-existing hearing loss, age, race/ethnicity sex, and radiation exposure. The interactions between treatment and baseline variables will be tested. Criteria for significance: Alpha=0.05, 95% confidence interval.

TABLE 11
ABBREVIATIONS
The list below includes abbreviations utilized in this protocol.

| | |
|---|---|
| AE | Adverse Event |
| ALT | Alanine Aminotransferase |
| apo B | Apolipoprotein B |
| AST | Aspartate Aminotransferase |
| AI | Associate Investigator |
| BUN | Blood Urea Nitrogen |
| CRF | Case Report Form |
| CRT | Chemoradiationtherapy |
| CD | Clinical Director |
| CLIA | Clinical Laboratory Improvement Amendments |
| CMP | Clinical Monitoring Plan |
| CFR | Code of Federal Regulations |
| CTCAEv5.0 | Common Terminology Criteria for Adverse |
| | Events, ver5.0 |
| CI | Confidence Interval |
| CHF | Congestive Heart Failure |
| CHD | Coronary Heart Disease |
| Cr | Creatine |
| CK | Creatine Kinase |
| CPK | Creatine Phosphokinase |
| DCC | Data Coordinating Center |
| dB | Decibel |
| DoH | Declaration of Helsinki |
| daPa | Dekapascal |
| DLT | Dose Limiting Toxicity |
| eCRF | Electronic Case Report Form |
| eCTD | Electronic Clinical Trials Data Base |
| FWA | Federalwide Assurance |
| FDA | Food and Drug Administration |
| GCP | Good Clinical Practice |
| GLP | Good Laboratory Practices |
| GMP | Good Manufacturing Practices |
| HNSCC | Head and Neck Squamous Cell Carcinoma |
| HIPPA | Health Insurance Portability and Accountability Act |
| HDL-C | High-Density Lipoprotein Cholesterol |
| HIV | Human Immunodeficiency Virus |
| HRPP | Human Research Protection Program |
| HMG-CoA | Hydroxymethylglutaryl-CoEnzymeA |
| IRB | Institutional Review Board |
| iRIS | Integrated Research Informational System |
| IMRT | Intensity-modulated Radiotherapy |
| ITT | Intention-to-treat |
| ICH GCP | International Conference on Harmonisation Good |
| | Clinical Practice |
| IQR | Interquartile Range |
| IDE | Investigational Device Exemption |
| IND | Investigational New Drug Application |
| kHz | Kilohertz |
| LDH | Lactate Dehydrogenase |
| LAI | Lead Associate Investigator |
| LDL-C | Low-Density Lipoprotein Cholesterol |
| MOP | Manual of Procedures |
| MedDRA | Medical Dictionary for Regulatory Activities |
| mg | Milligram |
| ml | Milliliter |
| mo | Month |
| MI | Myocardial Infarction |
| NCI | National Cancer Institute |
| NIDCD | National Institute on Deafness and Other |
| NIDCD | Communication Disorders |
| N | Number |
| PII | Personally Identifiable Information |
| PI | Principal Investigator |
| PTA | Pure Tone Audiometry |
| QA | Quality Assurance |
| QC | Quality Control |
| SAE | Serious Adverse Event |
| SGOT | Serum Glutamic-oxaloacetic Transaminase |
| SOP | Standard Operating Procedures |
| SOC | System Organ Class |
| TG | Triglyceride |
| UP | Unanticipated Problem |
| U.S. | United States |
| ULN | Upper Limit of Normal |
| yrs | Years |

Having described the invention in detail and by reference to specific embodiments thereof, it will be apparent that modifications and variations are possible without departing from the scope of the invention defined in the appended claims. More specifically, although some aspects of the instant invention are identified herein as particularly advantageous, it is contemplated that the instant invention is not necessarily limited to these particular aspects of the invention.

### REFERENCES

1. Adams, Stephen P., Michael Tsang, and James M. Wright. “Atorvastatin for Lowering Lipids.” Cochrane Database of Systematic Reviews, no. 3 (2015).
2. Adams S P, Tsang M, Wright J M. Atorvastatin for lowering lipids. Cochrane Database Syst Rev [Internet]. 2015 [cited 2020 Jan. 6];(3). Available from:

https://www.cochranelibrary.com/cdsr/doi/10.1002/14651858.CD008226.pub3/full
3. Adhyaru, Bhavin B., and Terry A. Jacobson. “Safety and Efficacy of Statin Therapy.” Nature Reviews Cardiology 15, no. 12 (December 2018): 757-69.
4. Ahmadvand, Anahita, Ameneh Yazdanfar, Fatemeh Yasrebifar, Younes Mohammadi, and Reza Mahjub and Maryam Mehrpooya *. “Evaluating the Effects of Oral and Topical Simvastatin in the Treatment of Acne Vulgaris: A Double-Blind, Randomized, Placebo-Controlled Clinical Trial.” Current Clinical Pharmacology, Oct. 31, 2018.
5. American National Standards Institute/Acoustical Society of America (ANSI/ASA) S3.6. Specifications for Audiometers. American National Standards Institute; 2018.
6. Azemawah, Veronica, Mohammad Reza Movahed, Patrick Centuori, Ryan Penaflor, Pascal L. Riel, Steven Situ, Mehrdad Shadmehr, and Mehrnoosh Hashemzadeh. “State of the Art Comprehensive Review of Individual Statins, Their Differences, Pharmacology, and Clinical Implications.” Cardiovascular Drugs and Therapy 33, no. 5 (October 2019): 625-39.
7. Aznaouridis K, Masoura C, Vlachopoulos C, Tousoulis D. Statins in Stroke | Bentham Science. Curr Med Chem. 2019; 26(33):6174-85.
8. Baker, Tiffany G., Soumen Roy, Carlene S. Brandon, Inga K. Kramarenko, Shimon P. Francis, Mona Taleb, Keely M. Marshall, Reto Schwendener, Fu-Shing Lee, and Lisa L. Cunningham. “Heat Shock Protein-Mediated Protection Against Cisplatin-Induced Hair Cell Death.” Journal of the Association for Research in Otolaryngology 16, no. 1 (Feb. 1, 2015): 67-80.
9. Bakker-Arkema, R. G., Davidson, M. H., Goldstein, R. J., Davignon, J., Isaacsohn, J. L., Weiss, S. R., Keilson, L. M., Brown, W. V., Miller, V. T., Shurzinske, L. J., Black, D. M., 1996. Efficacy and Safety of a New HMG-COA Reductase Inhibitor, Atorvastatin, in Patients With Hypertriglyceridemia. JAMA 275, 128-133. https://doi.org/10.1001/jama. 1996.03530260042029
10. Bao J, Sun B, Ma P, Gai Y, Sun W, Yu H, et al. Rosuvastatin alleviates myocardial fibrosis. Eur Rev Med Pharmacol Sci. 2018; 22(1):238-45.
11. Barbosa C P, Bracht L, Ames F Q, de Souza Silva-Comar F M, Tronco R P, Bersani-Amado C A. Effects of Ezetimibe, Simvastatin, and their Combination on Inflammatory Parameters in a Rat Model of Adjuvant-Induced Arthritis. Inflammation. 2017 Apr. 1; 40(2):717-24.
12. Beckwitt C H, Brufsky A, Oltvai Z N, Wells A. Statin drugs to reduce breast cancer recurrence and mortality. Breast Cancer Res BCR [Internet]. 2018 [cited 2020 Apr. 20];20. Available from: https://www.ncbi.nlm.nih.gov/pmc/articles/PMC6247616/13.

Bhandare N, Jackson A, Eisbruch A, Pan C C, Flickinger J C, Antonelli P, et al. Radiation therapy and hearing loss. Int J Radiat Oncol Biol Phys. 2010 Mar. 1; 76(3 Suppl): S50-57.
14. Beckwitt C H, Brufsky A, Oltvai Z N, Wells A. Statin drugs to reduce breast cancer recurrence and mortality. Breast Cancer Res 2018; 20:144.
15. Bertolini P, Lassalle M, Mercier G, Raquin M A, Izzi G, Corradini N, et al. Platinum Compound-Related Ototoxicity in Children: Long-Term Follow-Up Reveals Continuous Worsening of Hearing Loss. J Pediatr Hematol Oncol. 2004 October; 26(10):649-55.
16. Bifulco, Maurizio, Anna Maria Malfitano, and Giuseppe Marasco. “Potential Therapeutic Role of Statins in Neurological Disorders.” Expert Review of Neurotherapeutics 8, no. 5 (May 1, 2008): 827-37.
17. Bluethmann, Shirley M., Angela B. Mariotto, and Julia H. Rowland. “Anticipating the ‘Silver Tsunami’: Prevalence Trajectories and Co-Morbidity Burden Among Older Cancer Survivors in the United States.” Cancer Epidemiology, Biomarkers & Prevention: A Publication of the American Association for Cancer Research, Cosponsored by the American Society of Preventive Oncology 25, no. 7 (July 2016): 1029-36.
18. Brand, Yves, Cristian Setz, Soledad Levano, Alwin Listyo, Eduardo Chavez, Kwang Pak, Michael Sung, Vesna Radojevic, Allen F Ryan, and Daniel Bodmer. “Simvastatin Protects Auditory Hair Cells from Gentamicin-Induced Toxicity and Activates Akt Signaling in Vitro.” BMC Neuroscience 12 (Nov. 14, 2011): 114.
19. Brock P, Bellman S, Yeomans E, Pinkerton C, Pritchard J. Cisplatin ototoxicity in children: a practical grading system.-PubMed-NCBI. Med Pediatr Oncol. 1991; 19(4):295-300. doi: 10.1002/mpo.2950190415.
20. Brooks, Beth, and Kristin Knight. “Ototoxicity Monitoring in Children Treated with Platinum Chemotherapy.” International Journal of Audiology 57, no. sup4 (Aug. 24, 2018): S62-68.
21. Brungart, Douglas, Jaclyn Schurman, Dawn Konrad-Martin, Kelly Watts, Jay Buckey, Odile Clavier, Peter G. Jacobs, Samuel Gordon, and Marilyn F. Dille. “Using Tablet-Based Technology to Deliver Time-Efficient Ototoxicity Monitoring.” International Journal of Audiology 57, no. sup4 (Aug. 24, 2018): S78-86.
22. Cancer of the Oral Cavity and Pharynx-Cancer Stat Facts [WWW Document], n.d. . SEER. URL https://seer.cancer.gov/statfacts/html/oralcav.html (accessed Feb. 28, 2020).
23. Chang, Ning-Chia, Ming-Lung Yu, Kuen-Yao Ho, and Chi-Kung Ho. “Hyperlipidemia in Noise-Induced Hearing Loss.” Otolaryngology-Head and Neck Surgery 137, no. 4 (Oct. 1, 2007): 603-6.
24. Chen, Yafei, Xiaoli Li, Rui Zhang, Yuhong Xia, Zhuo Shao, and Zubing Mei. “Effects of Statin Exposure and Lung Cancer Survival: A Meta-Analysis of Observational Studies.” Pharmacological Research 141 (Mar. 1, 2019): 357-65.
25. Chu, Yu-Hsuan, Martha Sibrian-Vazquez, Jorge O. Escobedo, Amanda R. Phillips, D. Thomas Dickey, Qi Wang, Martina Ralle, Peter S. Steyger, and Robert M. Strongin. “Systemic Delivery and Biodistribution of Cisplatin in Vivo.” Molecular Pharmaceutics 13, no. 8 (Aug. 1, 2016): 2677-82.
26. Christou, Theodore, Hesham R. Omar, and Roger Dimitrov. “Periodic Rosuvastatin or Atorvastatin Dosing Arrays (PRADA): Patient-Centered Practice.” Drugs in R&D 14, no. 4 (December 2014): 221-25.
27. Ciorba, Andrea, Chiara Bianchini, Stefano Pelucchi, and Antonio Pastore. “The Impact of Hearing Loss on the Quality of Life of Elderly Adults.” Clinical Interventions in Aging 7 (2012): 159-63.
28. Collins R, Armitage J, Parish S, Sleight P, Peto R, Heart Protection Study Collaborative Group. MRC/BHF Heart Protection Study of cholesterol lowering with simvastatin in 20 536 high-risk individuals: a randomised placebocontrolled trial. The Lancet. 2002 Jul. 6; 360(9326):7-22.
29. “Common Terminology Criteria for Adverse Events (CTCAE) | Protocol Development | CTEP.” Accessed Dec. 29, 2019.
30. Coradini P P, Cigana L, Selistre S G A, Rosito L S, Brunetto A L. Ototoxicity From Cisplatin Therapy in Childhood Cancer. J Pediatr Hematol Oncol. 2007 June; 29(6):355-360.
31. Cucchiara B, Kasner S E. Use of statins in CNS disorders. J Neurol Sci. 2001 Jun. 15; 187(1):81-9
32. Dalton, Dayna S., Karen J. Cruickshanks, Barbara E. K. Klein, Ronald Klein, Terry L. Wiley, and David M. Nondahl. “The Impact of Hearing Loss on Quality of Life in Older Adults.” The Gerontologist 43, no. 5 (October 2003): 661-68.
33. Davies, R. A., 2016. Audiometry and other hearing tests. Handb. Clin. Neurol. 137, 157-176. https://doi.org/10.1016/B978-0-444-63437-5.00011-X
34. Dhakal, Sudip, Mishal Subhan, Joshua M. Fraser, Kenneth Gardiner, and lan Macreadie. “Simvastatin Efficiently Reduces Levels of Alzheimer's Amyloid Beta in Yeast.” International Journal of Molecular Sciences 20, no. 14 (Jul. 19, 2019).
35. Dille, Marilyn F., Debra Wilmington, Garnett P. McMillan, Wendy Helt, Stephen A. Fausti, and Dawn Konrad-Martin. “Development and Validation of a Cisplatin Dose-Ototoxicity Model.” Journal of the American Academy of Audiology 23, no. 7 (2012): 510-21.
36. Faul, F., Erdfelder, E., Buchner, A., Lang, A.-G., 2009. Statistical power analyses using G*Power 3.1: tests for correlation and regression analyses. Behav. Res. Methods 41, 1149-1160. https://doi.org/10.3758/BRM.41.4.1149
37. Faul, F., Erdfelder, E., Lang, A.-G., Buchner, A., 2007. G*Power 3: a flexible statistical power analysis program for the social, behavioral, and biomedical sciences. Behav. Res. Methods 39, 175-191. https://doi.org/10.3758/bf03193146
38. Fernandez K, Spielbauer K, Rusheen A, Wang L, Baker T, Eyles Stephen, et al. Lovastatin protects against cisplatin-induced hearing loss in mice. Hear Res. 2020 Apr. 1; 389:107905.
39. Fernandez K, Wafa T, Fitzgerald T S, Cunningham L L. An optimized, clinically relevant mouse model of cisplatin-induced ototoxicity. Hear Res. 2019 Apr. 1; 375:66-74.
40. Fernandez, K., Spielbauer, K., Rusheen, A., Wang, L., Baker, T., Eyles Stephen, Cunningham, L., 2020. Lovastatin protects against cisplatin-induced hearing loss in mice. Hear. Res. 389, 107905. https://doi.org/10.1016/j.heares.2020.107905
41. Fitzmaurice G M, Laird N M, Ware J H. Applied Longitudinal Analysis. John Wiley & Sons; 2012. 742 pp.
42. Fleming S, Ratanatharathorn V M D b, Weaver A M D c, Peters G, Peppard S M D d, Schumacher M R A b, et al. Ototoxicity from cis-platinum in patients with Stages III and IV previously untreated squamous cell cancer of the head and neck *. J Clin Oncol. 1985 August; 8(4):302-6.
43. Freyer, David R., Lu Chen, Mark D. Krailo, Kristin Knight, Doojduen Villaluna, Bonnie Bliss, Brad H. Pollock, et al. “Effects Of Sodium Thiosulfate Versus Observation On Development Of Cisplatin-Induced Hearing Loss In Children With Cancer: Results From The Children's Oncology Group Accl0431 Randomised Clinical Trial.” The Lancet. Oncology 18, no. 1 (January 2017): 63-74.
44. Frisina R D, Wheeler H E, Fossa S D, Kerns S L, Fung C, Sesso H D, et al. Comprehensive Audiometric Analysis of Hearing Impairment and Tinnitus After Cisplatin-Based Chemotherapy in Survivors of Adult-Onset Cancer. J Clin Oncol. 2016 Aug. 10; 34(23):2712-20.
45. Ghattas, Antonio Edmond, and João Pimenta. “Efficacy of Atorvastatin When Not Administered Daily.” Arquivos Brasileiros de Cardiologia 89, no. 5 (November 2007): 325-32.
46. Gopinath B, Flood V M, Teber E, McMahon C M, Mitchell P. Dietary Intake of Cholesterol Is Positively Associated and Use of Cholesterol-Lowering Medication Is Negatively Associated with Prevalent Age-Related Hearing Loss. J Nutr. 2011 Jul. 1; 141(7): 1355-61.
47. Gupta A, Stokes W, Eguchi M, Hararah M, Amini A, Mueller A, Morgan R, Bradley C, Raben D, McDermott J, Karam S D. Statin use associated with improved overall and cancer specific survival in patients with head and neck cancer. Oral Oncol 2019; 90:54-66.
48. Hameed, Mirza Khizer, Zeeshan Ayub Sheikh, Azeema Ahmed, and Atif Najam. “Atorvastatin in the Management of Tinnitus with Hyperlipidemias.” Journal of the College of Physicians and Surgeons—Pakistan: JCPSP 24, no. 12 (December 2014): 927-30.
49. Heine, C., and C. J. Browning. “Communication and Psychosocial Consequences of Sensory Loss in Older Adults: Overview and Rehabilitation Directions.” Disability and Rehabilitation 24, no. 15 (Jan. 1, 2002): 763-73.
50. li, Masaaki, and Douglas W. Losordo. “Statins and the Endothelium.” Vascular Pharmacology 46, no. 1 (Jan. 1, 2007): 1-9.
51. Jahani, L., A. H. Mehrparvar, M. Esmailidehaj, M. E. Rezvani, B. Moghbelolhossein, and Z. Razmjooei. “The Effect of Atorvastatin on Preventing Noise-Induced Hearing Loss: An Experimental Study.” The International Journal of Occupational and Environmental Medicine 7, no. 1 (2016): 15-21.
52. Jain, Mukesh K., and Paul M. Ridker. “Anti-Inflammatory Effects of Statins: Clinical Evidence and Basic Mechanisms.” Nature Reviews Drug Discovery 4, no. 12 (December 2005): 977-87.
53. Jian-Yu E, Graber J M, Lu S E, Lin Y, Lu-Yaho G, Tan X L. Effect of metformin and statin use on survival in pancreatic cancer patients: A systematic literature review and meta-analysis. Curr Med Chem 2018; 25:2595-2607.
54. Jones, Peter, Stephanie Kafonek, Irene Laurora, and Donald Hunninghake. “Comparative Dose Efficacy Study of Atorvastatin Versus Simvastatin, Pravastatin, Lovastatin, and Fluvastatin in Patients With Hypercholesterolemia (The CURVES Study) Fn1fn1This Study Was Supported by Parke-Davis, Division of Warner Lambert Company, Morris Plains, New Jersey. Manuscript Received Aug. 20, 1997; Revised Manuscript Received and Accepted Nov. 24, 1997.” The American Journal of Cardiology 81, no. 5 (Mar. 1, 1998): 582-87.
55. Knight K R, Chen L, Freyer D, Aplenc R, Bancroft M, Bliss B, et al. Group-Wide, Prospective Study of Ototoxicity Assessment in Children Receiving Cisplatin Chemotherapy (ACCL05C1): A Report From the Children's Oncology Group. J Clin Oncol. 2017 Feb. 1; 35(4):440-5.
56. Konrad-Martin D, Poling G L, Garinis A C, Ortiz C E, Hopper J, Bennett K O, et al. Applying U.S. national guidelines for ototoxicity monitoring in adult patients: Perspectives on patient populations, service gaps, barriers and solutions. Int J Audiol. 2018 September; 57(SUP4):S3-18.
57. Köppen, C., O. Reifschneider, I. Castanheira, M. Sperling, U. Karst, and G. Ciarimboli. “Quantitative Imaging of Platinum Based on Laser Ablation-Inductively Coupled Plasma-Mass Spectrometry to Investigate Toxic Side Effects of Cisplatin.” Metallomics 7, no. 12 (Dec. 2, 2015): 1595-1603.
58. Kwok, Simon C. M., Solomon P. Samuel, and John Handal. “Atorvastatin Activates Heme Oxygenase-1 at the Stress Response Elements.” Journal of Cellular and Molecular Medicine 16, no. 2 (2012): 394-400.
59. Lebo N L, Griffiths R, Hall S, Dimitroulakos J, Johnson-Obaseki S. Effect of statin use on oncologic outcomes in head and neck squamous cell carcinoma. Head Neck 2018; 40:1697-1706.
60. Lee H, Lee S, Lee H, Chung M, Park J, Park S, et al. Statin Use and Its Impact on Survival in Pancreatic Cancer Patients. Medicine (Baltimore) [Internet]. 2016 May [cited 2020 Jan. 6];95(19). Available from: insights.ovid.com
61. Lee Tzong-Shyuan, Chang Chih-Chieh, Zhu Yi, and Shyy John Y.-J. “Simvastatin Induces Heme Oxygenase-1.” Circulation 110, no. 10 (Sep. 7, 2004): 1296-1302.
62. Liao, James K. “Clinical Implications for Statin Pleiotropy.” Current Opinion in Lipidology 16, no. 6 (December 2005): 624.
63. Lin, David W., and Dennis R. Trune. “Breakdown of Stria Vascularis Blood-Labyrinth Barrier in C3H/Lpr Autoimmune Disease Mice.” Otolaryngology-Head and Neck Surgery 117, no. 5 (Nov. 1, 1997): 530-34.
64. Malfitano, Anna Maria, Giuseppe Marasco, Maria Chiara Proto, Chiara Laezza, Patrizia Gazzerro, and Maurizio Bifulco. “Statins in Neurological Disorders: An Overview and Update.” Pharmacological Research 88 (October 2014): 74-83.
65. Marnitz S, Schermeyer L, Dommerich S, Köhler C, Olze H, Budach V, et al. Age-corrected hearing loss after chemoradiation in cervical cancer patients. Strahlenther Onkol. 2018 Nov. 1; 194(11): 1039-48.
66. Miller, Kimberly D., Leticia Nogueira, Angela B. Mariotto, Julia H. Rowland, K. Robin Yabroff, Catherine M. Alfano, Ahmedin Jemal, Joan L. Kramer, and Rebecca L. Siegel. “Cancer Treatment and Survivorship Statistics, 2019.” CA: A Cancer Journal for Clinicians 69, no. 5 (2019): 363-85.
67. Mohammadkhani, Niloufar, Sedigheh Gharbi, Huda Fatima Rajani, Avishan Farzaneh, Golnoosh Mahjoob, Afsaneh Hoseinsalari, and Eberhard Korsching. “Statins: Complex Outcomes but Increasingly Helpful Treatment Options for Patients.” European Journal of Pharmacology 863 (Nov. 15, 2019): 172704.
68. “MRC/BHF Heart Protection Study of Cholesterol Lowering with Simvastatin in 20 536 High-Risk Individuals: A Randomised Placebocontrolled Trial.” The Lancet 360, no. 9326 (Jul. 6, 2002): 7-22.
69. Mukherjea, Debashree, and Leonard P Rybak. “Pharmacogenomics of Cisplatin-Induced Ototoxicity.” Pharmacogenomics 12, no. 7 (Jul. 1, 2011): 1039-50.
70. National Cancer Institute (NCI). Common Terminology Criteria for Adverse Events (CTCAE) | Protocol Development | CTEP [Internet]. 2017 [cited 2019 Dec. 29]. Available from: https://ctep.cancer.gov/protocolDevelopment/electronic_applications/ctc.htm #ctc_50
71. Neng, Lingling, Jinhui Zhang, Ju Yang, Fei Zhang, Ivan A. Lopez, Mingmin Dong, and Xiaorui Shi. “Structural Changes in Thestrial Blood-Labyrinth Barrier of Aged C57BL/6 Mice.” Cell and Tissue Research 361, no. 3 (Sep. 1, 2015): 685-96.
72. Oikonomou, Evangelos, Gerasimos Siasos, Marina Zaromitidou, George Hatzis, Konstantinos Mourouzis, Christine Chrysohoou, Konstantinos Zisimos, et al. “Atorvastatin Treatment Improves Endothelial Function through Endothelial Progenitor Cells Mobilization in Ischemic Heart Failure Patients.” Atherosclerosis 238, no. 2 (Feb. 1, 2015): 159-64.
73. Olzowy, Bernhard, Martin Canis, John-Martin Hempel, and Birgit Mazurek. “Effect of Atorvastatin on Progression of Sensorineural Hearing Loss and Tinnitus in the Elderly: Results of a Prospective, Randomized, Double-Blind Clinical Trial” 28, no. 4 (2007): 4.
74. Onusko, E. M., 2004. Tympanometry. Am. Fam. Physician 70, 1713-1720.
75. Park, J. S., S. W. Kim, K. Park, Y. H. Choung, I. Jou, and S. M. Park. “Pravastatin Attenuates Noise-Induced Cochlear Injury in Mice.” Neuroscience 208 (Apr. 19, 2012): 123-32.
76. Parke-Davis. Lipitor (atorvastatin calcium) Label. New York, NY: Pfizer Inc; 2009.
77. Power, Melinda C., Jennifer Weuve, A. Richey Sharrett, Deborah Blacker, and Rebecca F. Gottesman. “Statins, Cognition, and Dementia-Systematic Review and Methodological Commentary.” Nature Reviews Neurology 11, no. 4 (April 2015): 220-29.
78. Prayuenyong, Pattarawadee, Anand V. Kasbekar, and David M. Baguley. “The Efficacy of Statins as Otoprotective Agents: A Systematic Review.” Clinical Otolaryngology 2020; 45(1):21-31.
79. Prestes R, Daniela G. Impact of tinnitus on quality of life, loudness and pitch match, and high-frequency audiometry. Int Tinnitus J. 2009; 15(2): 134-8.
80. Rademaker-Lakhai, Jeany M., Mirjam Crul, Lot Zuur, Paul Baas, Jos H. Beijnen, Yvonne J. W. Simis, Nico van Zandwijk, and Jan H.M. Schellens. “Relationship Between Cisplatin Administration and the Development of Ototoxicity.” Journal of Clinical Oncology 24, no. 6 (Feb. 20, 2006): 918-24.
81. Richter C-P, Young H, Richter S V, Smith-Bronstein V, Stock S R, Xiao X, et al. Fluvastatin protects cochleae from damage by high-level noise. Sci Rep. 2018 14; 8(1):3033.
82. Ridker, Paul M., Christopher P. Cannon, David Morrow, Nader Rifai, Lynda M. Rose, Carolyn H. McCabe, Marc A. Pfeffer, and Eugene Braunwald. “C-Reactive Protein Levels and Outcomes after Statin Therapy.” Research-article. http://dx.doi.org/10.1056/NEJMoa042378, Oct. 8, 2009.
83. Rieke, Catherine, Odile Clavier, Lindsay Allen, Allison Anderson, Chris Brooks, Abigail Fellows, Douglas Brungart, and Jay Buckey. “Fixed-Level Frequency Threshold Testing for Ototoxicity Monitoring.” Ear and Hearing 38, no. 6 (December 2017).
84. Robertson, Margaret S., Susan S. Hayashi, Miranda L. Camet, Kathryn Trinkaus, Jennifer Henry, and Robert J. Hayashi. “Asymmetric Sensorineural Hearing Loss Is a Risk Factor for Late-Onset Hearing Loss in Pediatric Cancer Survivors Following Cisplatin Treatment.” Pediatric Blood & Cancer 66, no. 1 (2019): e27494.
85. Rodrigues G, Moreira A J, Bona S, Schemitt E, Marroni C A, Di Naso F C, et al. Simvastatin Reduces Hepatic Oxidative Stress and Endoplasmic Reticulum Stress in Nonalcoholic Steatohepatitis Experimental Model. Oxid Med Cell Longev [Internet]. 2019 Jun. 18 [cited 2020 Feb. 28]; 2019. Available from: https://www.ncbi.nlm.nih.gov/pmc/articles/PMC6604429/86. Rolland, Viannique, François Meyer, Matthieu J. Guitton, Richard Bussières, Daniel Philippon, Isabelle Bairati, Mathieu Leclerc, and Mathieu Côté. “A Randomized Controlled Trial to Test the Efficacy of Trans-Tympanic Injections of a Sodium Thiosulfate Gel to Prevent Cisplatin-Induced Ototoxicity in Patients with Head and Neck Cancer.” Journal of Otolaryngology-Head & Neck Surgery 48, no. 1 (Jan. 16, 2019): 4.
87. Ruijven, Marjolein W. M. van, John C. M. J. de Groot, Sjaak F. L. Klis, and Guido F. Smoorenburg. “The Cochlear Targets of Cisplatin: An Electrophysiological and Morphological Time-Sequence Study.” Hearing Research 205, no. 1 (Jul. 1, 2005): 241-48.
88. Saeedi Saravi S S, Saeedi Saravi S S, Arefidoust A, Dehpour A R. The beneficial effects of HMG-CoA reductase inhibitors in the processes of neurodegeneration. Metab Brain Dis. 2017 Aug. 1; 32(4): 949-65.
89. Saadah, H. A., 1993. Vestibular Vertigo Associated With Hyperlipidemia: Response to Antilipidemic Therapy. Arch. Intern. Med. 153, 1846-1849. https://doi.org/10. 1001/archinte. 1993.00410150136014
90. Schell, M J, V A McHaney, A A Green, L E Kun, F A Hayes, M Horowitz, and W H Meyer. “Hearing Loss in Children and Young Adults Receiving Cisplatin with or without Prior Cranial Irradiation.” Journal of Clinical Oncology 7, no. 6 (Jun. 1, 1989): 754-60.
91. Schuette, Andrew, Daniel P. Lander, Dorina Kallogjeri, Cathryn Collopy, Sneha Goddu, Tanya M. Wildes, Mackenzie Daly, and Jay F. Piccirillo. “Predicting Hearing Loss After Radiotherapy and Cisplatin Chemotherapy in Patients With Head and Neck Cancer.” JAMA Otolaryngology—Head & Neck Surgery, Nov. 21, 2019.
92. Scobioala, Sergiu, Ross Parfitt, Peter Matulat, Christopher Kittel, Fatemeh Ebrahimi, Heidi Wolters, Antoinette am Zehnhoff-Dinnesen, and Hans Theodor Eich. “Impact of Radiation Technique, Radiation Fraction Dose, and Total Cisplatin Dose on Hearing.” Strahlentherapie Und Onkologie 193, no. 11 (Nov. 1, 2017): 910-20.
93. Seckl M J, Ottensmeier C H, Cullen M, Schmid P, Ngai Y, Muthukumar D, Thompson J, Harden S, Middleton G, Fife K M, Crosse B, Taylor P, Nash S, Hackshaw A. Multicenter, Phase III, randomized, double-blind, placebo-controlled trial of pravastatin added to first-line standard chemotherapy in small-cell lung cancer (LUNGSTAR). J Clin Oncol 2017; 35:1506-1514.
94. Shen, Z., Li, S., Sheng, B., Shen, Q., Sun, L.-Z., Zhu, H., Zhu, X., 2018. The role of atorvastatin in suppressing tumor growth of uterine fibroids. J. Transl. Med. 16. https://doi.org/10.1186/s12967-018-1430-x
95. Shi, Xiaorui. “Cochlear Pericyte Responses to Acoustic Trauma and the Involvement of Hypoxia-Inducible Factor-1a and Vascular Endothelial Growth Factor | Elsevier Enhanced Reader.” The American Journal of Pathophysiology 174, no. 5 (May 2009): 1692-1704.
96. Shi X. “Pathophysiology of the Cochlear Intrastrial Fluid-Blood Barrier (Review).” Hearing Research, Special Issue: Annual Reviews 2016, 338 (Aug. 1, 2016): 52-63.
97. Sirtori, Cesare R. “The Pharmacology of Statins.” Pharmacological Research, Statin: New Life for an Old Drug, 88 (Oct. 1, 2014): 3-11.
98. Sparks D L, Sabbagh M N, Connor D J, Lopez J, Launer L J, Browne P, et al. Atorvastatin for the treatment of mild to moderate Alzheimer disease: preliminary results. Arch Neurol. 2005 May; 62(5):753-7.
99. Stone, Neil J., Jennifer G. Robinson, Alice H. Lichtenstein, C. Noel Bairey Merz, Conrad B. Blum, Robert H. Eckel, Anne C. Goldberg, et al. “2013 ACC/AHA Guideline on the Treatment of Blood Cholesterol to Reduce Atherosclerotic Cardiovascular Risk in Adults: A Report of the American College of Cardiology/American Heart Association Task Force on Practice Guidelines.” Journal of the American College of Cardiology 63, no. 25, Part B (Jul. 1, 2014): 2889-2934.
100. Sutbas A, Yetiser S, Satar B, Akcam T, Karahatay S, Saglam K. Low-cholesterol diet and antilipid therapy in managing tinnitus and hearing loss in patients with noise-induced hearing loss and hyperlipidemia. Int Tinnitus J. 2007; 13(2): 143-9.
101. Syka, Josef, Ladislav Ouda, Petr Nachtigal, Dagmar Solichová, and Vladimír Semecky. “Atorvastatin Slows down the Deterioration of Inner Ear Function with Age in Mice.” Neuroscience Letters 411, no. 2 (Jan. 10, 2007): 112-16.
102. The Otitis Media Guideline Panel, 1994. Managing Otitis Media With Effusion in Young Children. Pediatrics 94, 766-773.
103. Theunissen, Eleonoor A. R., Wouter A. Dreschler, Merel N. Latenstein, Coen R. N. Rasch, Sieberen van der Baan, Jan Paul de Boer, Alfons J. M. Balm, and Charlotte L. Zuur. “A New Grading System for Ototoxicity in Adults.” Annals of Otology, Rhinology & Laryngology 123, no. 10 (Oct. 1, 2014): 711-18.
104. Theunissen E A R, Bosma S C J, Zuur C L, Spijker R, Baan S van der, Dreschler W A, et al. Sensorineural hearing loss in patients with head and neck cancer after chemoradiotherapy and radiotherapy: A systematic review of the literature. Head Neck. 2015; 37(2):281-92.
105. Thiesen, Signe, Peng Yin, Andrea L Jorgensen, Jieying Eunice Zhang, Valentina Manzo, Laurence McEvoy, Christopher Barton, et al. “TPMT, COMT and ACYP2 Genetic Variants in Paediatric Cancer Patients with Cisplatin-Induced Ototoxicity.” Pharmacogenetics and Genomics 27, no. 6 (June 2017): 213-22.
106. Thompson, Gregory P., Douglas P. Sladen, Becky J. Hughes Borst, and Owen L. Still. “Accuracy of a Tablet Audiometer for Measuring Behavioral Hearing Thresholds in a Clinical Population.” Otolaryngology-Head and Neck Surgery 153, no. 5 (Nov. 1, 2015): 838-42.
107. Ung, Matthew H., Todd A. Mackenzie, Tracy L. Onega, Christopher I. Amos, and Chao Cheng. “Statins Associated with Improved Mortality among Patients with Certain Histological Subtypes of Lung Cancer.” Lung Cancer (Amsterdam, Netherlands) 126 (December 2018): 89-96.
108. Ung, M. H., Mackenzie, T. A., Onega, T. L., Amos, C. I., Cheng, C., 2018a. Statins associated with improved mortality among patients with certain histological subtypes of lung cancer. Lung Cancer Amst. Neth. 126, 89-96. https://doi.org/10.1016/j.lungcan.2018.10.022
109. Ung, M. H., Mackenzie, T. A., Onega, T. L., Amos, C. I., Cheng, C., 2018b. Statins associate with improved mortality among patients with certain histological subtypes of lung cancer. Lung Cancer Amst. Neth. 126, 89-96. https://doi.org/10.1016/j.lungcan.2018.10.022
110. US Department of Health and Human Services, National Institutes of Health, National Cancer Institute. Common Terminology Criteria for Adverse Events (CTCAE) Version 5.0. https://ctep.cancer.gov/protocolDevelopment/electronic_applications/docs/CT CAE_v5_Quick_Reference_5 x7.pdf. Updated Nov. 27, 2017. Accessed Jul. 17, 2018.
111. Weng, Tingwen, Erin E Devine, Hongming Xu, Zhisong Yan, and Pin Dong. “A Clinical Study of Serum Lipid Disturbance in Chinese Patients with Sudden Deafness.” Lipids in Health and Disease 12 (Jul. 3, 2013): 95.
112. Whitlon, Donna S., Mary Grover, Sara F. Dunne, Sonja Richter, Chi-Hao Luan, and Claus-Peter Richter. “Novel High Content Screen Detects Compounds That Promote Neurite Regeneration from Cochlear Spiral Ganglion Neurons.” Scientific Reports 5 (Nov. 2, 2015).
113. Xie W, Ning L, Huang Y, Liu Y, Zhang W, Hu Y, Yang J. Statin use and survival outcomes in endocrine-related gynecologic cancers: A systematic review and meta-analysis. Oncotarget 2017; 8:41508-41517.
114. Yücel, Hilal, Abitter Yücel, Hamdi Arbağ, Erkan Cure, Mehmet Akif Eryilmaz, and Ahmet Bedri Ozer. “Effect of Statins on Hearing Function and Subjective Tinnitus in Hyperlipidemic Patients.” Romanian Journal of Internal Medicine=Revue Roumaine De Medecine Interne 57, no. 2 (Jun. 1, 2019): 133-40.
115. Zhang Y, Wang Y-T, Koka S, Zhang Y, Hussain T, Li X. Simvastatin improves lysosome function via enhancing lysosome biogenesis in endothelial cells. Front Biosci Landmark Ed. 2020 Jan. 1; 25:283-98.
116. Zuur, Charlotte L., Yvonne J. Simis, Pauline E. Lansdaal, Augustinus A. Hart, Coen R. Rasch, Jan H. Schornagel, Wouter A. Dreschler, and Alfons J. Balm. “Risk Factors of Ototoxicity After Cisplatin-Based Chemo-Irradiation in Patients With Locally Advanced Head-and-Neck Cancer: A Multivariate Analysis.” International Journal of Radiation Oncology*Biology*Physics 68, no. 5 (Aug. 1, 2007): 1320-25.

1. A method of reducing or preventing drug-induced hearing loss in an individual receiving the drug, or intended to receive the drug, the method comprising administering to the individual a compound that possesses one or more pharmacological activities of atorvastatin, and/or wherein the compound possesses one or more pharmacokinetic parameters substantially similar to one or more pharmacokinetic parameters of atorvastatin.

2. The method of claim 1, wherein the one or more pharmacokinetic parameters is/are selected from the group consisting of the ability to cross a blood barrier, lipophilicity, half-life, potency, bioavailability, absorption, and excretion.

3. The method of claim 2, wherein the blood-barrier separates the inner ear from peripheral blood.

4. The method of claim 1, wherein the one or more pharmacological activities is/are selected from the group consisting of inhibition of hydroxymethylglutaryl-CoA (HMG-COA) reductase, modulation of endothelial function, reduction of inflammation, induction of heme oxygenase-1 (Hmox-1, and promotion of elongation of spiral ganglion neurons (SGN)).

5. The method of claim 1, wherein the compound inhibits hydroxymethylglutaryl-CoA (HMG-COA) reductase.

6. The method of claim 1, wherein the compound is a statin, or a functional derivative thereof.

7. (canceled)

8. The method of claim 1, wherein the compound is selected from the group consisting of atorvastatin, fluvastatin, lovastatin, pravastatin, rosuvastatin, simvastatin, pitavastatin, and functional derivatives thereof.

9. The method of claim 1, wherein the compound is atorvastatin, or a functional derivative thereof.

10. The method of claim 1, wherein the individual has, or is suspected of having, cancer.

11. The method of claim 10, wherein the cancer is a cancer of the head or neck.

12. The method of claim 1, wherein the drug is selected from the group consisting of a non-steroidal anti-inflammatory agent, an antibiotic, a chemotherapeutic agent, a diuretic, and a quinine-based compound.

13. The method of claim 1, wherein the drug is a chemotherapeutic agent.

14. The method of claim 1, wherein the drug possesses one or more activities of cisplatin or carboplatin.

15. The method of claim 1, wherein the drug is cisplatin, carboplatin, or a functional derivative thereof.

16. The method of claim 1, wherein the compound is administered at a time prior to the individual receiving a first administration of the drug.

17. The method of claim 1, wherein the compound is administered during the time period the individual is administered the drug.

18. The method of claim 1, wherein reducing hearing loss comprises reducing the threshold shift.

19. A kit for reducing or preventing drug-induced hearing loss in an individual receiving the drug, or intended to receive the drug, the kit comprising: (1) a compound that reduces or prevents the drug-induced hearing loss, wherein the compound possesses one or more pharmacological activities of atorvastatin, and/or wherein the compound comprises one or more pharmacokinetic parameters substantially similar to one or more pharmacokinetic parameters of atorvastatin; and (2) instructions for administering the compound to the individual.

20-33. (canceled)

34. A method of using a compound that possesses one or more pharmacological activities of atorvastatin, and/or that possesses one or more pharmacokinetic parameters substantially similar to one or more pharmacokinetic parameters of atorvastatin, in the preparation of a medicament for reducing or preventing drug-induced hearing loss in an individual.

35-47. (canceled)

48. The method of claim 1, wherein the compound is administered at a dosage of approximately 10 to approximately 80 mg/day.

49-52. (canceled)

53. The method of claim 1, wherein the compound comprises one or more of a 10 mg atorvastatin dosage unit, a 20 mg atorvastatin dosage unit, a 40 mg atorvastatin dosage unit, and an 80 mg atorvastatin dosage unit.

54. The method of claim 48, wherein the dosage form is a tablet or a capsule.

55. The method of claim 54, wherein the dosage form is a gelatin capsule.
